(19) United States
(12) Patent Application Publication
(10) Pub. No.: US 2021/0388053 A1
(43) Pub. Date: Dec. 16, 2021

(54) Fc Variants with Altered Binding to Neonatal Fc Receptor (FCRN) for Veterinary Use

(71) Applicant: Kindred Biosciences, Inc., Burlingame, CA (US)

(72) Inventors: Hangjun Zhan, Foster City, CA (US); Lam Nguyen, Union City, CA (US); Qingyi Chu, Burlingame, CA (US); Richard Chin, San Francisco, CA (US); Shyr Jiann Li, Millbrae, CA (US)

(73) Assignee: Kindred Biosciences, Inc., Burlingame, CA (US)

(21) Appl. No.: 17/284,875

(22) PCT Filed: Oct. 18, 2019
(86) PCT No.: PCT/US2019/057093

§ 371 Date: Apr. 13, 2021

Related U.S. Application Data
(60) Provisional application No. 62/747,613, filed on Oct. 18, 2018.
(60) Provisional application No. 62/809,715, filed on Feb. 24, 2019.

(51) Int. Cl. C07K 14/705

(52) U.S. Cl. CPC C07K 14/70503; A61K 38/00

(57) ABSTRACT

Provided are various embodiments relating to variant IgG Fc polypeptides with altered FcRn binding activity. Also provided are polypeptides and pharmaceutical compositions comprising the variant IgG Fc polypeptides described herein. Polypeptides comprising variant IgG Fc polypeptides of the present invention may have extended half-life in vivo. Such products may be used in methods to treat disease in companion animals, such as canines, felines, and equines.

This application claims the benefit of U.S. Provisional Application No. 62/747,613, filed Oct. 18, 2018, and U.S. Provisional Application No. 62/809,715, filed Feb. 24, 2019, each of which is incorporated by reference herein in its entirety for any purpose.

### FIELD

This present disclosure relates to variant IgG Fc polypeptides of companion animals with altered binding affinity to FcRn, such as enhanced binding affinity to FcRn at acidic pH (e.g., at a pH in the range of from about 5.0 to about 6.5). The variant IgG Fc polypeptides of the present disclosure may extend the half-life or improve pharmacokinetics of an antibody or IgG Fc fusion protein in vivo. In addition, variant IgG Fc polypeptides may be used in the design and production of antibodies or fusion proteins for treating various disorders in companion animals.

### BACKGROUND

The neonatal Fc receptor (FcRn) is an Fc receptor that is similar in structure to the MHC class I molecules. Like MHC class I molecules, FcRn also associates with beta-2-microglobulin (B2M). FcRn is understood to facilitate transcytosis and recycling of IgG in vivo, and hence increase the in vivo half-life of IgG compared to that of other antibody isotypes.

To improve the binding affinity of IgG to FcRn at an acid pH (e.g., at a pH in the range of from about 5.0 to about 6.5), the Fc region of IgG responsible for binding to FcRn may be modified through mutagenesis.

Companion species animals, such as cats, dogs, and horses, have species specific IgG Fc sequences. Furthermore, there are multiple IgG subclasses, each having Fc sequence differences. For example, canine has IgG-A, IgG-B, IgG-C and IgG-D.

### SUMMARY

Embodiment 1. A polypeptide comprising a variant IgG Fc polypeptide comprising at least one amino acid substitution relative to a wild-type IgG Fc polypeptide derived from a companion animal species, wherein the variant Fc polypeptide is capable of binding to neonatal Fc receptor (FcRn) with an increased affinity relative to the wild-type Fc polypeptide.

Embodiment 2. The polypeptide of embodiment 1, wherein the variant Fc polypeptide binds to FcRn with an affinity greater than the wild-type IgG Fc polypeptide, as measured by biolayer interferometry, surface plasmon resonance, or any protein-protein interaction tool at a pH in the range of from about 5.0 to about 6.5, such as at a pH of about 5.0, a pH of about 5.2, a pH of about 5.5, a pH of about 6.0, a pH of about 6.2, or a pH of about 6.5.

Embodiment 3. The polypeptide of embodiment 1 or embodiment 2, wherein the variant IgG Fc polypeptide binds to FcRn with a dissociation constant (Kd) of less than 5×10⁻⁶ M, less than 1×10⁻⁶ M, less than 5×10⁻⁷ M, less than 1×10⁻⁷ M, less than 5×10⁻⁸ M, less than 1×10⁻⁸M, less than 5×10⁻⁹ M, less than 1×10⁻⁹M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry, surface plasmon resonance, or any protein-protein interaction tool at a pH in the range of from about 5.0 to about 6.5, such as at a pH of about 5.0, a pH of about 5.5, a pH of about 6.0, or a pH of about 6.5.

Embodiment 4. The polypeptide of any one of the preceding embodiments, wherein the polypeptide has increased serum half-life relative to a polypeptide comprising a wild-type Fc.

Embodiment 5. The polypeptide of any one of the preceding embodiments, wherein the companion animal species is canine, feline, or equine.

Embodiment 6. The polypeptide of any one of the preceding embodiments, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1 Fc, or IgG2 Fc.

Embodiment 7. The polypeptide of any one of the preceding embodiments, wherein the variant IgG Fc polypeptide is at least 90% identical, at least 95% identical, at least 97% identical, or at least 99% identical to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID N: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, or SEQ ID NO: 91.

Embodiment 8. The polypeptide of any one of the preceding embodiments, wherein the variant IgG Fc polypeptide comprises:
a) at least one amino acid substitution at at least one position corresponding to position 9, 18, 22, 23, 24, 25, 26, 27, 28, 30, 31, 60, 79, 80, 83, 132, 149, 162, 183, 203, 205, and/or 209 of SEQ ID NO: 1;
b) at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 2;
c) at least one amino acid substitution at at least one position corresponding to position 9, 18, 22, 23, 24, 26, 27, 28, 30, 31, 60, 79, 80, 81, 83, 132, 148, 162, 182, 202, 204, and/or 208 of SEQ ID NO: 3;
d) at least one amino acid substitution at at least one position corresponding to position 9, 18, 22, 23, 24, 25, 26, 27, 28, 30, 60, 79, 80, 83, 132, 149, 163, 183, 203, 205, and/or 209 of SEQ ID NO: 4;
e) at least one amino acid substitution at at least one position corresponding to position 9, 18, 22, 23, 24, 25, 26, 27, 28, 30, 31, 79, 80, 81, 83, 132, 148, 162, 182, 202, 204, 208, and/or 213 of SEQ ID NO: 5;
f) at least one amino acid substitution at at least one position corresponding to position 9, 18, 22, 23, 24, 25, 26, 27, 28, 79, 80, 81, 83, 132, 148, 162, 182, 202, 204, 208, and/or 213 of SEQ ID NO: 6;
g) at least one amino acid substitution at at least one position corresponding to position 9, 18, 22, 23, 24, 25, 27, 28, 30, 31, 60, 79, 80, 81, 83, 132, 148, 162, 182, 202, 204, 208, and/or 213 of SEQ ID NO: 7;
h) at least one amino acid substitution at at least one position corresponding to position 9, 18, 22, 23, 24, 25, 27, 28, 30, 31, 79, 80, 83, 132, 148, 162, 182, 202, 204, 208, and/or 213 of SEQ ID NO: 8;
i) at least one amino acid substitution at at least one position corresponding to position 9, 18, 22, 23, 24, 25, 27, 28, 30, 31, 79, 80, 81, 83, 132, 148, 162, 182, 202, 204, 208 and/or 213 of SEQ ID NO: 9;
j) at least one amino acid substitution at at least one position corresponding to position 18, 22, 23, 24, 25, 27, 28, 30, 31, 79, 80, 81, 83, 132, 148, 162, 182, 202, 204, 208, and/or 213 of SEQ ID NO: 10;
k) at least one amino acid substitution at at least one position corresponding to position 18, 22, 23, 24, 25, 27, 28, 30, 31, 79, 80, 81, 83, 132, 148, 162, 182, 202, 204, 208, and/or 213 of SEQ ID NO: 11;
l) at least one amino acid substitution at at least one position corresponding to position 9, 18, 22, 23, 24, 25, 26, 27, 28, 30, 31, 60, 79, 80, 81, 83, 148, 162, 182, 202, 204, 208, and/or 213 of SEQ ID NO: 12;
m) at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 13; or
n) at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 54.

Embodiment 9. The polypeptide of any one of the preceding embodiments, wherein the variant IgG Fc polypeptide comprises:
a) at least one amino acid substitution at at least one of position selected from 9, 18, 22, 23, 24, 25, 26, 27, 28, 30, 31, 60, 79, 80, 83, 132, 149, 162, 183, 203, 205, and/or 209 of SEQ ID NO: 1;
b) at least one amino acid substitution at at least one of position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 2;
c) at least one amino acid substitution at at least one of position selected from position 9, 18, 22, 23, 24, 26, 27, 28, 30, 31, 60, 79, 80, 81, 83, 132, 148, 162, 182, 202, 204, and/or 208 of SEQ ID NO: 3;
d) at least one amino acid substitution at at least one of position selected from position 9, 18, 22, 23, 24, 25, 26, 27, 28, 30, 60, 79, 80, 83, 132, 149, 163, 183, 203, 205, and/or 209 of SEQ ID NO: 4;
e) at least one amino acid substitution at at least one of position selected from position 9, 18, 22, 23, 24, 25, 26, 27, 28, 30, 31, 79, 80, 81, 83, 132, 148, 162, 182, 202, 204, 208, and/or 213 of SEQ ID NO: 5;
f) at least one amino acid substitution at at least one of position selected from position 9, 18, 22, 23, 24, 25, 26, 27, 28, 79, 80, 81, 83, 132, 148, 162, 182, 202, 204, 208, and/or 213 of SEQ ID NO: 6;
g) at least one amino acid substitution at at least one of position selected from position 9, 18, 22, 23, 24, 25, 27, 28, 30, 31, 60, 79, 80, 81, 83, 132, 148, 162, 182, 202, 204, 208, and/or 213 of SEQ ID NO: 7;
h) at least one amino acid substitution at at least one of position selected from position 9, 18, 22, 23, 24, 25, 27, 28, 30, 31, 79, 80, 83, 132, 148, 162, 182, 202, 204, 208, and/or 213 of SEQ ID NO: 8;
i) at least one amino acid substitution at at least one of position selected from position 9, 18, 22, 23, 24, 25, 27, 28, 30, 31, 79, 80, 81, 83, 132, 148, 162, 182, 202, 204, 208 and/or 213 of SEQ ID NO: 9;
j) at least one amino acid substitution at at least one of position selected from position 18, 22, 23, 24, 25, 27, 28, 30, 31, 79, 80, 81, 83, 132, 148, 162, 182, 202, 204, 208, and/or 213 of SEQ ID NO: 10;
k) at least one amino acid substitution at at least one of position selected from position 18, 22, 23, 24, 25, 27, 28, 30, 31, 79, 80, 81, 83, 132, 148, 162, 182, 202, 204, 208, and/or 213 of SEQ ID NO: 11;
l) at least one amino acid substitution at at least one of position selected from position 9, 18, 22, 23, 24, 25, 26, 27, 28, 30, 31, 60, 79, 80, 81, 83, 148, 162, 182, 202, 204, 208, and/or 213 of SEQ ID NO: 12;
m) at least one amino acid substitution at at least one of position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 13; or
n) at least one amino acid substitution at at least on position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 54.

Embodiment 10. The polypeptide of any one of the preceding embodiments, wherein the variant IgG Fc polypeptide comprises:
a) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 149; a leucine, a phenylalanine, or a tyrosine at a position corresponding to position 162; an isoleucine at a position corresponding to position 183; a leucine or a tryptophan at a position corresponding to position 203; a proline at a position corresponding to position 205; and/or a serine, an alanine, an arginine, a phenylalanine, a tyrosine, a histidine, or a tryptophan at a position corresponding to position 209 of SEQ ID NO: 1;
b) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, tyrosine, methionine, arginine, or valine at a position corresponding to position 24; a threonine at a position corresponding to position 25; a tryptophan, a tyrosine, a histidine, a leucine, a phenylalanine, a valine, a methionine, a threonine, or a serine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 2;
c) a tyrosine at a position corresponding to position 9; a proline at a position corresponding to position 18; a histidine or a proline at a position corresponding to position 22; a valine, an arginine, or a lysine at a position corresponding to position 23; a phenylalanine, a tyrosine, a methionine, or an arginine at a position corresponding to position 24; a tryptophan, a tyrosine, a histidine, a leucine, a phenylalanine, a valine, a methionine, a threonine, or a serine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; and/or a serine, an alanine, an arginine, a phenylalanine, a tyrosine, a histidine, or a tryptophan at a position corresponding to position 208 of SEQ ID NO: 3;
d) a tyrosine at a position corresponding to position 9; a proline at a position corresponding to position 18; a histidine or a proline at a position corresponding to position 22; a valine, an arginine, or a lysine at a position corresponding to position 23; a phenylalanine, a tyrosine, a methionine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an alanine at a position corresponding to position 60; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 149; a leucine, a phenylalanine, or a tyrosine at a position corresponding to position 163; an isoleucine at a position corresponding to position 183; a leucine or a tryptophan at a position corresponding to position 203; a proline at a position corresponding to position 205; and/or a serine, an alanine, an arginine, a phenylalanine, a tyrosine, a histidine, or a tryptophan at a position corresponding to position 209 of SEQ ID NO: 4;
e) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, a glutamic acid, or a methionine at a position corresponding to position 79, a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 5;
f) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, a methionine, or a threonine at a position corresponding to position 26; a leucine, a methionine, a proline, an isoleucine, an asparagine, or a valine at a position corresponding to position 27; a methionine, a glutamic acid, an asparagine, a histidine, an isoleucine, or a valine at a position corresponding to position 28; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83, an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 6;
g) a tyrosine at a position corresponding to position 9; a proline at a position corresponding to position 18; a histidine, an isoleucine, and/or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, a glutamic acid, or a methionine at a position corresponding to position 79, a histidine or a phenylalanine at a position corresponding to position 80; a glutamic acid at a position corresponding to position 81; an alanine, a tyrosine, a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 7;
h) a tyrosine at a position corresponding to position 9; a proline at a position corresponding to position 18; a histidine, an isoleucine, or a proline at a position corresponding to position 22; a valine, an arginine, a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 8;
i) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 9;
j) a proline at a position corresponding to position 18; a histidine, an isoleucine, or a proline at a position corresponding to position 22; a valine, an arginine, or a lysine at a position corresponding to position 23; a phenylalanine, a tyrosine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, or a tryptophan at a position corresponding to position 208; and/or an isoleucine or a valine at a position corresponding to position 213 of SEQ ID NO: 10;
k) a proline at a position corresponding to position 18; a histidine, an isoleucine, or a proline at a position corresponding to position 22; a valine, an arginine, or a lysine at a position corresponding to position 23; a phenylalanine, a tyrosine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 11;
l) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 12;
m) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 13; or n) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 54.

Embodiment 11. The polypeptide of any one of the preceding embodiments, wherein the variant IgG Fc polypeptide comprises:
a) a tyrosine at position 9; a proline at position 18; a histidine, an isoleucine, or a proline at position 22; a valine, an arginine, or a lysine at position 23; a phenylalanine, a tyrosine, a methionine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 149; a leucine, a phenylalanine, or a tyrosine at position 162; an isoleucine at position 183; a leucine or a tryptophan at position 203; a proline at position 205; and/or a serine, an alanine, an arginine, a phenylalanine, a tyrosine, a histidine, or a tryptophan at position 209 of SEQ ID NO: 1;
b) a tyrosine at position 9; a proline at position 18; a histidine, an isoleucine, or a proline at position 22; a valine, an arginine, or a lysine at position 23; a phenylalanine, tyrosine, methionine, arginine, or valine at position 24; a threonine at position 25; a tryptophan, a tyrosine, a histidine, a leucine, a phenylalanine, a valine, a methionine, a threonine, or a serine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 2;
c) a tyrosine at position 9; a proline at position 18; a histidine or a proline at position 22; a valine, an arginine, or a lysine at position 23; a phenylalanine, a tyrosine, a methionine, or an arginine at position 24; a tryptophan, a tyrosine, a histidine, a leucine, a phenylalanine, a valine, a methionine, a threonine, or a serine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; and/or a serine, an alanine, an arginine, a phenylalanine, a tyrosine, a histidine, or a tryptophan at position 208 of SEQ ID NO: 3;
d) a tyrosine at position 9; a proline at position 18; a histidine or a proline at position 22; a valine, an arginine, or a lysine at position 23; a phenylalanine, a tyrosine, a methionine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an alanine at position 60; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 149; a leucine, a phenylalanine, or a tyrosine at position 163; an isoleucine at position 183; a leucine or a tryptophan at position 203; a proline at position 205; and/or a serine, an alanine, an arginine, a phenylalanine, a tyrosine, a histidine, or a tryptophan at position 209 of SEQ ID NO: 4;
e) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, a glutamic acid, or a methionine at position 79, a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 5;
f) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, a methionine, or a threonine at position 26; a leucine, a methionine, a proline, an isoleucine, an asparagine, or a valine at position 27; a methionine, a glutamic acid, an asparagine, a histidine, an isoleucine, or a valine at position 28; a glutamine, an arginine, an alanine, glutamic acid, or a methionine at position 79; a histidine or a phenylalanine at position 80; a glutamic acid at position 81; an alanine, a tyrosine, or a phenylalanine at position 83, an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 6;
g) a tyrosine at position 9; a proline at position 18; a histidine, an isoleucine, and/or a proline at position 22; a valine, an arginine, or a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 7;
h) a tyrosine at position 9; a proline at position 18; a histidine, an isoleucine, or a proline at position 22; a valine, an arginine, a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 8;
i) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 9;
j) a proline at position 18; a histidine, an isoleucine, or a proline at position 22; a valine, an arginine, or a lysine at position 23; a phenylalanine, a tyrosine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, or a tryptophan at position 208; and/or an isoleucine or a valine at position 213 of SEQ ID NO: 10;
k) a proline at position 18; a histidine, an isoleucine, or a proline at position 22; a valine, an arginine, or a lysine at position 23; a phenylalanine, a tyrosine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, a glutamic acid, or a methionine at position 79, a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, a phenylalanine, a tyrosine, a histidine, or a tryptophan at position 208; and/or an isoleucine or a valine at position 213 of SEQ ID NO: 11;
l) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; a valine at position 148; a leucine, a phenylalanine, or a tyrosine at position 162; an isoleucine at position 182; a leucine or a tryptophan at position 202; a proline at position 204; an alanine, an arginine, a phenylalanine, a tyrosine, a histidine, or a tryptophan at position 208; and/or an isoleucine or a valine at position 213 of SEQ ID NO: 12;
m) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or tryptophan at position 202; a proline at position 204; an alanine, an arginine, a phenylalanine, a tyrosine, a histidine, or a tryptophan at position 208; and/or an isoleucine or a valine at position 213 of SEQ ID NO: 13; or
n) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or tryptophan at position 202; a proline at position 204; an alanine, an arginine, a phenylalanine, a tyrosine, a histidine, or a tryptophan at position 208; and/or an isoleucine or a valine at position 213 of SEQ ID NO: 54.

Embodiment 12. The polypeptide of any one of the preceding embodiments, wherein the variant IgG Fc polypeptide comprises:
a) a phenylalanine or a tyrosine at a position corresponding to position 24, a threonine at a position corresponding to position 26, and/or a glutamic acid at a position corresponding to position 28 of SEQ ID NO: 1;
b) a phenylalanine at a position corresponding to position 24, a threonine at a position corresponding to position 26, and/or a glutamic acid at a position corresponding to position 28 of SEQ ID NO: 2, SEQ ID NO: 3, or SEQ ID NO: 4; or
c) a phenylalanine or a tyrosine at a position corresponding to position 24, a threonine at a position corresponding to position 26, and/or a glutamic acid at a position corresponding to position 28 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 13. The polypeptide of any one of the preceding embodiments, wherein the variant IgG Fc polypeptide comprises:
a) a phenylalanine or a tyrosine at position 24, a threonine at position 26, and/or a glutamic acid at position 28 of SEQ ID NO: 1;
b) a phenylalanine at position 24, a threonine at position 26, and/or a glutamic acid at position 28 of SEQ ID NO: 2, SEQ ID NO: 3, or SEQ ID NO: 4; or
c) a phenylalanine or a tyrosine at position 24, a threonine at position 26, and/or a glutamic acid at position 28 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 14. The polypeptide of any one of the preceding claims, wherein the variant IgG Fc polypeptide comprises a phenylalanine at a position corresponding to position 24 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 15. The polypeptide of any one of the preceding claims, wherein the variant IgG Fc polypeptide comprises a phenylalanine at position 24 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 16. The polypeptide of any one of the preceding embodiments, wherein the variant IgG Fc polypeptide comprises:
a) a tyrosine at a position corresponding to position 24, a tyrosine at a position corresponding to position 83, and/or an arginine, a phenylalanine, a tyrosine, a histidine, or a tryptophan at a position corresponding to position 209 of SEQ ID NO: 1 or SEQ ID NO: 4;
b) a tyrosine at a position corresponding to position 24, a tyrosine at a position corresponding to position 83, and/or an arginine, a phenylalanine, a tyrosine, a histidine, or a tryptophan at a position corresponding to position 208 of SEQ ID NO: 2 or SEQ ID NO: 3; or
c) a tyrosine at a position corresponding to position 24, a tyrosine at a position corresponding to position 83, and/or an arginine, a phenylalanine, a tyrosine, a histidine, or a tryptophan at a position corresponding to position 208 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 17. The polypeptide of any one of the preceding embodiments, wherein the variant IgG Fc polypeptide comprises:
a) a tyrosine at position 24, a tyrosine at position 83, and/or an arginine, a phenylalanine, a tyrosine, a histidine, or a tryptophan at position 209 of SEQ ID NO: 1 or SEQ ID NO: 4;
b) a tyrosine at position 24, a tyrosine at position 83, and/or an arginine, a phenylalanine, a tyrosine, a histidine, or a tryptophan at position 208 of SEQ ID NO: 2 or SEQ ID NO: 3; or
c) a tyrosine at position 24, a tyrosine at position 83, and/or an arginine, a phenylalanine, a tyrosine, a histidine, or a tryptophan at position 208 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 18. The polypeptide of any one of the preceding embodiments, wherein the variant IgG Fc polypeptide comprises:
a) a tyrosine at a position corresponding to position 24 and an arginine at a position corresponding to position 209 of SEQ ID NO: 1 or SEQ ID NO: 4;
b) a tyrosine at a position corresponding to position 24 and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
c) a tyrosine at a position corresponding to position 24 and a phenylalanine at a position corresponding to position 209 of SEQ ID NO: 1 or SEQ ID NO: 4;
d) a tyrosine at a position corresponding to position 24 and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
e) a tyrosine at a position corresponding to position 24 and a tyrosine at a position corresponding to position 209 of SEQ ID NO: 1 or SEQ ID NO: 4;
f) a tyrosine at a position corresponding to position 24 and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
g) a tyrosine at a position corresponding to position 24 and a histidine at a position corresponding to position 209 of SEQ ID NO: 1 or SEQ ID NO: 4;
h) a tyrosine at a position corresponding to position 24 and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
i) a tyrosine at a position corresponding to position 24 and a tryptophan at a position corresponding to position 209 of SEQ ID NO: 1 or SEQ ID NO: 4;
j) a tyrosine at a position corresponding to position 24 and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
k) a tyrosine at a position corresponding to position 83 and an arginine at a position corresponding to position 209 of SEQ ID NO: 1 or SEQ ID NO: 4;
l) a tyrosine at a position corresponding to position 83 and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
m) a tyrosine at a position corresponding to position 83 and a phenylalanine at a position corresponding to position 209 of SEQ ID NO: 1 or SEQ ID NO: 4;
n) a tyrosine at a position corresponding to position 83 and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
o) a tyrosine at a position corresponding to position 83 and a tyrosine at a position corresponding to position 209 of SEQ ID NO: 1 or SEQ ID NO: 4;
p) a tyrosine at a position corresponding to position 83 and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
q) a tyrosine at a position corresponding to position 83 and a histidine at a position corresponding to position 209 of SEQ ID NO: 1 or SEQ ID NO: 4;
r) a tyrosine at a position corresponding to position 83 and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
s) a tyrosine at a position corresponding to position 83 and a tryptophan at a position corresponding to position 209 of SEQ ID NO: 1 or SEQ ID NO: 4;
t) a tyrosine at a position corresponding to position 83 and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
u) a tyrosine at a position corresponding to position 24, a tyrosine at a position corresponding to position 83, and an arginine at a position corresponding to position 209 of SEQ ID NO: 1 or SEQ ID NO: 4;
v) a tyrosine at a position corresponding to position 24, a tyrosine at a position corresponding to position 83, and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
w) a tyrosine at a position corresponding to position 24, a tyrosine at a position corresponding to position 83, and a phenylalanine at a position corresponding to position 209 of SEQ ID NO: 1 or SEQ ID NO: 4;
x) a tyrosine at a position corresponding to position 24, a tyrosine at a position corresponding to position 83, and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
y) a tyrosine at a position corresponding to position 24, a tyrosine at a position corresponding to position 83, and a tyrosine at a position corresponding to a position corresponding to position 209 of SEQ ID NO: 1 or SEQ ID NO: 4;
z) a tyrosine at a position corresponding to position 24, a tyrosine at a position corresponding to position 83, and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
aa) a tyrosine at a position corresponding to position 24, a tyrosine at a position corresponding to position 83, and a histidine at a position corresponding to position 209 of SEQ ID NO: 1 or SEQ ID NO: 4;
bb) a tyrosine at a position corresponding to position 24, a tyrosine at a position corresponding to position 83, and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
cc) a tyrosine at a position corresponding to position 24, a tyrosine at a position corresponding to position 83, and a tryptophan at a position corresponding to position 209 of SEQ ID NO: 1 or SEQ ID NO: 4; or
dd) a tyrosine at a position corresponding to position 24, a tyrosine at a position corresponding to position 83, and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13; or SEQ ID NO: 54.

Embodiment 19. The polypeptide of any one of the preceding embodiments, wherein the variant IgG Fc polypeptide comprises:
a) a tyrosine at position 24 and an arginine at position 209 of SEQ ID NO: 1 or SEQ ID NO: 4;
b) a tyrosine at position 24 and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
c) a tyrosine at position 24 and a phenylalanine at position 209 of SEQ ID NO: 1 or SEQ ID NO: 4;
d) a tyrosine at position 24 and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
e) a tyrosine at position 24 and a tyrosine at position 209 of SEQ ID NO: 1 or SEQ ID NO: 4;
f) a tyrosine at position 24 and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
g) a tyrosine at position 24 and a histidine at position 209 of SEQ ID NO: 1 or SEQ ID NO: 4;
h) a tyrosine at position 24 and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
i) a tyrosine at position 24 and a tryptophan at position 209 of SEQ ID NO: 1 or SEQ ID NO: 4;
j) a tyrosine at position 24 and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
k) a tyrosine at position 83 and an arginine at position 209 of SEQ ID NO: 1 or SEQ ID NO: 4;
l) a tyrosine at position 83 and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
m) a tyrosine at position 83 and a phenylalanine at position 209 of SEQ ID NO: 1 or SEQ ID NO: 4;
n) a tyrosine at position 83 and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
o) a tyrosine at position 83 and a tyrosine at position 209 of SEQ ID NO: 1 or SEQ ID NO: 4;
p) a tyrosine at position 83 and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
q) a tyrosine at position 83 and a histidine at position 209 of SEQ ID NO: 1 or SEQ ID NO: 4;
r) a tyrosine at position 83 and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
s) a tyrosine at position 83 and a tryptophan at position 209 of SEQ ID NO: 1 or SEQ ID NO: 4;
t) a tyrosine at position 83 and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
u) a tyrosine at position 24, a tyrosine at position 83, and an arginine at position 209 of SEQ ID NO: 1 or SEQ ID NO: 4;
v) a tyrosine at position 24, a tyrosine at position 83, and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
w) a tyrosine at position 24, a tyrosine at position 83, and a phenylalanine at position 209 of SEQ ID NO: 1 or SEQ ID NO: 4;
x) a tyrosine at position 24, a tyrosine at position 83, and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
y) a tyrosine at position 24, a tyrosine at position 83, and a tyrosine at position 209 of SEQ ID NO: 1 or SEQ ID NO: 4;
z) a tyrosine at position 24, a tyrosine at position 83, and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
aa) a tyrosine at position 24, a tyrosine at position 83, and a histidine at position 209 of SEQ ID NO: 1 or SEQ ID NO: 4;
bb) a tyrosine at position 24, a tyrosine at position 83, and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
cc) a tyrosine at position 24, a tyrosine at position 83, and a tryptophan at position 209 of SEQ ID NO: 1 or SEQ ID NO: 4; or
dd) a tyrosine at position 24, a tyrosine at position 83, and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

Embodiment 20. The polypeptide of any one of the preceding embodiments, wherein the variant IgG Fc polypeptide comprises the amino acid sequence of SEQ ID NO: 14, SEQ ID NO: 15, SEQ ID NO: 16, SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, or SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, or SEQ ID NO: 91.

Embodiment 21. The polypeptide of any one of the preceding embodiments, wherein the polypeptide comprises the amino acid sequence of SEQ ID NO: 14, SEQ ID NO: 15, SEQ ID NO: 16 SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, SEQ ID NO: 51, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, SEQ ID NO: 91, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 108, SEQ ID NO: 109, SEQ ID NO: 110, SEQ ID NO: 113, SEQ ID NO: 114, SEQ ID NO: 115, SEQ ID NO: 120, SEQ ID NO: 121, SEQ ID NO: 126, SEQ ID NO: 127, SEQ ID NO: 128, or SEQ ID NO: 129.

Embodiment 22. The polypeptide of any one of the preceding embodiments, wherein the at least one amino acid substitution comprises an amino acid substitution with an amino acid derivative.

Embodiment 23. The polypeptide of embodiment 22, wherein the amino acid derivative is a histidine derivative, a tryptophan derivative, a tyrosine derivative, a leucine derivative, a phenylalanine derivative, a valine derivative, a methionine derivative an isoleucine derivative, an arginine derivative, a glutamic acid derivative, a proline derivative, a lysine derivative, a threonine derivative, an asparagine derivative, a glutamine derivative, a serine derivative, an alanine derivative, or a glutamic acid derivative.

Embodiment 24. The polypeptide of any one of the preceding embodiments, wherein the polypeptide is an antibody, an antibody fragment, or a fusion polypeptide.

Embodiment 25. The polypeptide of any one of the preceding embodiments, wherein the polypeptide comprises at least one therapeutic polypeptide selected from a late embryogenesis abundant (LEA) protein polypeptide (e.g., an LEA-3 polypeptide), an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ECD of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.

Embodiment 26. The polypeptide of embodiment 25, wherein the therapeutic polypeptide is a canine polypeptide, a feline polypeptide, or an equine polypeptide.

Embodiment 27. The polypeptide of any one of the preceding embodiments, wherein the polypeptide comprises an antibody or antibody fragment that binds at least one target polypeptide selected from a late embryogenesis abundant (LEA) protein polypeptide (e.g., an LEA-3 polypeptide), an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Ra2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.

Embodiment 28. The polypeptide of embodiment 27, wherein the target polypeptide is a canine polypeptide, a feline polypeptide, or an equine polypeptide.

Embodiment 29. The polypeptide of any one of embodiments 1 to 24, wherein the polypeptide comprises an antibody or antibody fragment that binds canine or feline IL31.

Embodiment 30. The polypeptide of any one of the preceding embodiments, wherein the variant IgG Fc polypeptide has altered binding affinity to Protein A, C1q, CD16, CD32, or CD64 relative to the wild-type IgG Fc polypeptide.

Embodiment 31. A polypeptide comprising the amino acid sequence of SEQ ID NO: 14, SEQ ID NO: 15, SEQ ID NO: 16 SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, SEQ ID NO: 51, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, SEQ ID NO: 91, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 108, SEQ ID NO: 109, SEQ ID NO: 110, SEQ ID NO: 113, SEQ ID NO: 114, SEQ ID NO: 115, SEQ ID NO: 120, SEQ ID NO: 121, SEQ ID NO: 126, SEQ ID NO: 127, SEQ ID NO: 128, or SEQ ID NO: 129.

Embodiment 32. An isolated nucleic acid encoding the polypeptide of any one of the preceding embodiments.

Embodiment 33. A host cell comprising the nucleic acid of embodiment 32.

Embodiment 34. A host cell that expresses the polypeptide of any one of embodiments 1 to 31.

Embodiment 35. A method of producing a polypeptide comprising culturing the host cell of embodiment 33 or of embodiment 34 and isolating the polypeptide.

Embodiment 36. A pharmaceutical composition comprising the polypeptide of any one of embodiments 1 to 31 and a pharmaceutically acceptable carrier.

Embodiment 37. A method of delivering a polypeptide to a companion animal species comprising administering the polypeptide of any one of embodiments 1 to 31 or the pharmaceutical composition of embodiment 36 parenterally.

Embodiment 38. A method of delivering a polypeptide to a companion animal species comprising administering the polypeptide of any one of embodiments 1 to 31 or the pharmaceutical composition of embodiment 36 by an intramuscular route, an intraperitoneal route, an intracerebrospinal route, a subcutaneous route, an intra-arterial route, an intrasynovial route, an intrathecal route, or an inhalation route.

Embodiment 39. The method of embodiment 37 or embodiment 38, wherein the companion animal species is canine, equine, or feline.

### BRIEF DESCRIPTION OF THE DRAWINGS

FIG. 1A and FIG. 1B shows partial sequences of variant canine IgG Fc polypeptides having single mutations that were screened from twelve single site NNK mutation libraries for enhanced binding with canine FcRn.

FIGS. 2A, 2B, 2C, 2D, 2E, and 2F are graphs of binding assays for wild-type (FIG. 2A) and variant canine IgG-B Fc polypeptides with single mutations of L(24)Y (FIG. 2B); L(24)F (FIGS. 2C and 2D); L(24)M (FIG. 2E); and L(24)S (FIG. 2F). The polypeptides exhibited off rates (koff) at pH 6.0 of 1.22×10⁻¹ (wild-type); 1.38×10⁻² (L(24)Y); 6.31×10⁻² and 8.47×10⁻² (L(24)F); 1.26×10⁻¹ (L(24)M); and 2.41×10⁻¹ (L(24)S).

FIG. 3 shows a Biacore sensorgram of various concentrations of canine FcRn (12.5, 25, 50, 100, and 200 nM) binding to wild-type canine IgG-B Fc polypeptide.

FIG. 4 shows a Biacore sensorgram of various concentrations of canine FcRn (12.5, 25, 50, 100, and 200 nM) binding to variant canine IgG-B Fc polypeptide L(24)Y.

FIG. 5 shows a Biacore sensorgram of various concentrations of canine FcRn (12.5, 25, 50, 100, and 200 nM) binding to variant canine IgG-B Fc polypeptide L(24)F.

FIG. 6 shows a Biacore sensorgram of various concentrations of canine FcRn (12.5, 25, 50, 100, and 200 nM) binding to variant canine IgG-B Fc polypeptide L(24)M.

FIG. 7 shows a Biacore sensorgram of various concentrations of canine FcRn (12.5, 25, 50, 100, and 200 nM) binding to variant canine IgG-B Fc polypeptide YTE.

FIG. 8 shows partial sequences of variant canine IgG Fc polypeptides having two or more amino acid substitutions for enhanced binding with canine FcRn.

FIG. 9 is a plot of a steady state binding analysis of an antibody having a wild-type canine IgG-B Fc polypeptide with FcRn/B2M complex.

FIG. 10 is a plot of a steady state binding analysis of an antibody having a YTE variant canine IgG-B Fc polypeptide with FcRn/B2M complex.

FIG. 11 is a sensorgram of a kinetic binding analysis of an antibody having a Y0Y variant canine IgG-B Fc polypeptide with FcRn/B2M complex.

FIG. 12 is a sensorgram of a kinetic binding analysis of an antibody having a YYY variant canine IgG-B Fc polypeptide with FcRn/B2M complex.

FIG. 13 is a OctetRed sensorgram of feline FcRn binding with wild-type feline IgG1b (labeled as B) and YTE variant feline IgG1b (labeled as A) at low pH (pH 6.0) for association and neutral pH (pH 7.2) for dissociation.

FIG. 14 is a OctetRed sensorgram of chimeric variant canine IgG-A Fc FOO antibody (A) and IgG-D Fc F00 antibody (B) binding to canine FcRn compared to that of chimeric variant canine IgG-A Fc without the Phe mutation (C) and IgG-D Fc without the Phe mutation (D).

FIG. 15 shows the serum pharmacokinetics profiles for chimeric variant canine IgG-A F00 antibody (“IgG-A F00”; n=2) and chimeric variant canine IgG-A without the Phe mutation (“IgG-A”; n=2) after subcutaneous administration to rats at 2 mg/kg.

FIG. 16 is a OctetRed sensorgram of chimeric anti-TNFα antibodies with variant canine IgG-B Fcs (0Y0, 0YH, 0YY, or 00Y) binding to canine FcRn compared to that of chimeric anti-TNFα antibody with a wild-type canine IgG-B.

### DESCRIPTION OF CERTAIN SEQUENCES

Table 1 provides a listing of certain sequences referenced herein.

TABLE 1
Description of the Sequences

| SEQ ID | | |
|---|---|---|
| NO: | SEQUENCE | DESCRIPTION |
| 1 | CPVPEPLGGPSVLIFPPKPKDILRITRTPEVTCVVLDLGRE | Exemplary wild-type |
| | DPEVQISWFVDGKEVHTAKTQSREQQFNGTYRVVSVLPIEH | canine IgG-A Fc |
| | QDWLTGKEFKCRVNHIDLPSPIERTISKARGRAHKPSVYVL | |
| | PPSPKELSSSDTVSITCLIKDFYPPDIDVEWQSNGQQEPER | |
| | KHRMTPPQLDEDGSYFLYSKLSVDKSRWQQGDPFTCAVMHE | |
| | TLQNHYTDLSLSHSPGK | |
| 2 | CPAPEMLGGPSVFIFPPKPKDTLLIARTPEVTCVVVDLDPE | Exemplary wild-type |
| | DPEVQISWFVDGKQMQTAKTQPREEQFNGTYRVVSVLPIGH | canine IgG-B Fc |
| | QDWLKGKQFTCKVNNKALPSPIERTISKARGQAHQPSVYVL | |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHNHYTQESLSHSPGK | |
| 3 | CPGCGLLGGPSVFIFPPKPKDILVTARTPTVTCVVVDLDPE | Exemplary wild-type |
| | NPEVQISWFVDSKQVQTANTQPREEQSNGTYRVVSVLPIGH | canine IgG-C Fc |
| | QDWLSGKQFKCKVNNKALPSPIEEIISKTPGQAHQPNVYVL | |
| | PPSRDEMSKNTVTLTCLVKDFFPPEIDVEWQSNGQQEPESK | |
| | YRMTPPQLDEDGSYFLYSKLSVDKSRWQRGDTFICAVMHEA | |
| | LHNHYTQISLSHSPGK | |
| 4 | CPVPESLGGPSVFIFPPKPKDILRITRTPEITCVVLDLGRE | Exemplary wild-type |
| | DPEVQISWFVDGKEVHTAKTQPREQQFNSTYRVVSVLPIEH | canine IgG-D Fc |
| | QDWLTGKEFKCRVNHIGLPSPIERTISKARGQAHQPSVYVL | |
| | PPSPKELSSSDTVTLTCLIKDFFPPEIDVEWQSNGQPEPES | |
| | KYHTTAPQLDEDGSYFLYSKLSVDKSRWQQGDTFTCAVMHE | |
| | ALQNHYTDLSLSHSPGK | |
| 5 | CPAPELLGGPSVFIFPPNPKDTLMITRTPEVTCVVVDVSQE | Exemplary wild-type |
| | NPDVKFNWYMDGVEVRTATTRPKEEQFNSTYRVVSVLRIQH | equine IgG1 Fc |
| | QDWLSGKEFKCKVNNQALPQPIERTITKTKGRSQEPQVYVL | |
| | APHPDELSKSKVSVTCLVKDFYPPEINIEWQSNGQPELETK | |
| | YSTTQAQQDSDGSYFLYSKLSVDRNRWQQGTTFTCGVMHEA | |
| | LHNHYTQKNVSKNPGK | |
| 6 | YTHSKFLGGPSVFIFPPNPKDALMISRTPVVTCVVVNLSDQ | Exemplary wild-type |
| | YPDVQFSWYVDNTEVHSAITKQREAQFNSTYRVVSVLPIQH | equine IgG2 Fc |
| | QDWLSGKEFKCSVTNVGVPQPISRAISRGKGPSRVPQVYVL | |
| | PPHPDELAKSKVSVTCLVKDFYPPDISVEWQSNRWPELEGK | |
| | YSTTPAQLDGDGSYFLYSKLSLETSRWQQVESFTCAVMHEA | |
| | LHNHFTKTDISESLGK | |
| 7 | CPAPELLGGPSVFIFPPKPKDVLMITRTPEVTCLVVDVSHD | Exemplary wild-type |
| | SSDVLFTWYVDGTEVKTAKTMPNEEQNNSTYRVVSVLRIQH | equine IgG3 Fc |
| | QDWLNGKKFKCKVNNQALPAPVERTISKATGQTRVPQVYVL | |
| | APHPDELSKNKVSVTCLVKDFLPTDITVEWQSNEHPEPEGK | |
| | YRTTEAQKDSDGSYFLYSKLTVETDRWQQGTTFTCVVMHEA | |
| | LHNHVMQKNVSHSPGK | |
| 8 | CPAECLQVGPSVFIFPPKPKDVLMISRTPTVTCVVVDVGHD | Exemplary wild-type |
| | FPDVQFNWYVDGVETHTATTEPKQEQFNSTYRVVSVLPIQH | equine IgG4 Fc |
| | KDWLSGKEFKCKVNNKALPAPVERTISKPTGQPREPQVYVL | |
| | APHRDELSKNKVSVTCLVKDFYPTDIDIEWKSNGQPEPETK | |
| | YSTTPAQLDSDGSYFLYSKLTVETNRWQQGTTFTCAVMHEA | |
| | LHNHYTEKSVSKSPGK | |
| 9 | CPAPELPGGPSVFIFPPKPKDVLKISRKPEVTCVVVDLGHD | Exemplary wild-type |
| | DPDVQFTWFVDGVETHTATTEPKEEQFNSTYRVVSVLPIQH | equine IgG5 Fc |
| | QDWLSGKEFKCSVTNKALPAPVERTTSKAKGQLRVPQVYVL | |
| | APHPDELAKNTVSVTCLVKDFYPPEIDVEWQSNEHPEPEGK | |
| | YSTTPAQLNSDGSYFLYSKLSVETSRWKQGESFTCGVMHEA | |
| | VENHYTQKNVSHSPGK | |
| 10 | CCCPKCPGRPSVFIFPPNPKDTLMISRTPEVTCVVVDVSQE | Exemplary wild-type |
| | NPDVKFNWYVDGVEAHTATTKAKEKQDNSTYRVVSVLPIQH | equine IgG6 Fc |
| | QDWRRGKEFKCKVNNRALPAPVERTITKAKGELQDPKVYIL | |
| | APHREEVTKNTVSVTCLVKDFYPPDINVEWQSNEEPEPEVK | |
| | YSTTPAQLDGDGSYFLYSKLTVETDRWEQGESFTCVVMHEA | |
| | IRHTYRQKSITNFPGK | |
| 11 | TCPECLSVGPSVFIFPPKPKDVLMISRTPTVTCVVVDVGHD | Exemplary wild-type |
| | FPDVQFNWYVDGVETHTATTEPKQEQNNSTYRVVSILAIQH | equine IgG7 Fc |
| | KDWLSGKEFKCKVNNQALPAPVQKTISKPTGQPREPQVYVL | |
| | APHRDELSKNKVSVTCLVKDFYPTDIDIEWKSNGQPEPETK | |
| | YSTTPAQLDSDGSYFLYSKLTVETNRWQQGTTFTCAVMHEA | |
| | LHNHYTEKSVSKSPGK | |
| 12 | CPPPEMLGGPSIFIFPPKPKDTLSISRTPEVTCLVVDLGPD | Exemplary wild-type |
| | DSDVQITWFVDNTQVYTAKTSPREEQFNSTYRVVSVLPILH | feline IgG1a Fc |
| | QDWLKGKEFKCKVNSKSLPSPIERTISKDKGQPHEPQVYVL | |
| | PPAQEELSRNKVSVTCLIEGFYPSDIAVEWEITGQPEPENN | |
| | YRTTPPQLDSDGTYFLYSRLSVDRSRWQRGNTYTCSVSHEA | |
| | LHSHHTQKSLTQSPGK | |
| 13 | CPPPEMLGGPSIFIFPPKPKDTLSISRTPEVTCLVVDLGPD | Exemplary wild-type |
| | DSDVQITWFVDNTQVYTAKTSPREEQFNSTYRVVSVLPILH | feline IgG1b Fc |
| | QDWLKGKEFKCKVNSKSLPSPIERTISKAKGQPHEPQVYVL | |
| | PPAQEELSRNKVSVTCLIKSFHPPDIAVEWEITGQPEPENN | |
| | YRTTPPQLDSDGTYFVYSKLSVDRSHWQRGNTYTCSVSHEA | |
| | LHSHHTQKSLTQSPGK | |
| 54 | CPVPEIPGAPSVFIFPPKPKDTLSISRTPEVTCLVVDLGPD | Exemplary wild-type |
| | DSNVQITWFVDNTEMHTAKTRPREEQFNSTYRVVSVLPILH | feline IgG2 Fc |
| | QDWLKGKEFKCKVNSKSLPSAMERTISKAKGQPHEPQVYVL | |
| | PPTQEELSENKVSVTCLIKGFHPPDIAVEWEITGQPEPENN | |
| | YQTTPPQLDSDGTYFLYSRLSVDRSHWQRGNTYTCSVSHEA | |
| | LHSHHTQKSLTQSPGK | |
| 14 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (Y00) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHNHYTQESLSHSPGK | |
| 15 | CPAPEMLGGPSVFIFPPKPKDTLFIARTPEVTCVVVDLDPE | Exemplary variant canine |
| | DPEVQISWFVDGKQMQTAKTQPREEQFNGTYRVVSVLPIGH | IgG-B Fc (F00) |
| | QDWLKGKQFTCKVNNKALPSPIERTISKARGQAHQPSVYVL | Leu24Phe |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHNHYTQESLSHSPGK | |
| 16 | CPAPEMLGGPSVFIFPPKPKDTLMIARTPEVTCVVVDLDPE | Exemplary variant canine |
| | DPEVQISWFVDGKQMQTAKTQPREEQFNGTYRVVSVLPIGH | IgG-B Fc (M00) |
| | QDWLKGKQFTCKVNNKALPSPIERTISKARGQAHQPSVYVL | Leu24Met |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHNHYTQESLSHSPGK | |
| 17 | CPAPEMLGGPSVFIFPPKPKDTLSIARTPEVTCVVVDLDPE | Exemplary variant canine |
| | DPEVQISWFVDGKQMQTAKTQPREEQFNGTYRVVSVLPIGH | IgG-B Fc (S00) |
| | QDWLKGKQFTCKVNNKALPSPIERTISKARGQAHQPSVYVL | Leu24Ser |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHNHYTQESLSHSPGK | |
| 27 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (0Y0) |
| | YDWLKGKQFTCKVNNKALPSPIERTISKARGQAHQPSVYVL | Gln83Tyr |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHNHYTQESLSHSPGK | |
| 28 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (00R) |
| | QDWLKGKQFTCKVNNKALPSPIERTISKARGQAHQPSVYVL | Asn208Arg |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHRHYTQESLSHSPGK | |
| 29 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (00F) |
| | QDWLKGKQFTCKVNNKALPSPIERTISKARGQAHQPSVYVL | Asn208Phe |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHFHYTQESLSHSPGK | |
| 30 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (00Y) |
| | QDWLKGKQFTCKVNNKALPSPIERTISKARGQAHQPSVYVL | Asn208Tyr |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHYHYTQESLSHSPGK | |
| 31 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (00H) |
| | QDWLKGKQFTCKVNNKALPSPIERTISKARGQAHQPSVYVL | Asn208His |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHHHYTQESLSHSPGK | |
| 32 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (00W) |
| | QDWLKGKQFTCKVNNKALPSPIERTISKARGQAHQPSVYVL | Asn208Trp |
| | PPSREELSKNTVSLTCLIKDFFPPDIDVEWQSNGQQEPESK | |
| | YRTTPPQLDEDGSYFLYSKLSVDKSRWQRGDTFICAVMHEA | |
| | LHWHYTQESLSHSPGK | |
| 33 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (Y0R) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | Asn208Arg |
| | YRTTPPQLDEDGSYFLYSKLSVDKSRWQRGDTFICAVMHEA | |
| | LHRHYTQESLSHSPGK | |
| 34 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (Y0F) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | Asn208Phe |
| | YRTTPPQLDEDGSYFLYSKLSVDKSRWQRGDTFICAVMHEA | |
| | LHFHYTQESLSHSPGK | |
| 35 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (Y0Y) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | Asn208Tyr |
| | YRTTPPQLDEDGSYFLYSKLSVDKSRWQRGDTFICAVMHEA | |
| | LHYHYTQESLSHSPGK | |
| 36 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (Y0H) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | Asn208His |
| | YRTTPPQLDEDGSYFLYSKLSVDKSRWQRGDTFICAVMHEA | |
| | LHHHYTQESLSHSPGK | |
| 37 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (Y0W) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | Asn208Trp |
| | YRTTPPQLDEDGSYFLYSKLSVDKSRWQRGDTFICAVMHEA | |
| | LHWHYTQESLSHSPGK | |
| 38 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (0YR) |
| | YDWLKGKQFTCKVNNKALPSPIERTISKARGQAHQPSVYVL | Gln83Tyr |
| | PPSREELSKNTVSLTCLIKDFFPPDIDVEWQSNGQQEPESK | Asn208Arg |
| | YRTTPPQLDEDGSYFLYSKLSVDKSRWQRGDTFICAVMHEA | |
| | LHRHYTQESLSHSPGK | |
| 39 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (0YF) |
| | YDWLKGKQFTCKVNNKALPSPIERTISKARGQAHQPSVYVL | Gln83Tyr |
| | PPSREELSKNTVSLTCLIKDFFPPDIDVEWQSNGQQEPESK | Asn208Phe |
| | YRTTPPQLDEDGSYFLYSKLSVDKSRWQRGDTFICAVMHEA | |
| | LHFHYTQESLSHSPGK | |
| 40 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (0YY) |
| | YDWLKGKQFTCKVNNKALPSPIERTISKARGQAHQPSVYVL | Gln83Tyr |
| | PPSREELSKNTVSLTCLIKDFFPPDIDVEWQSNGQQEPESK | Asn208Tyr |
| | YRTTPPQLDEDGSYFLYSKLSVDKSRWQRGDTFICAVMHEA | |
| | LHYHYTQESLSHSPGK | |
| 41 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (0YH) |
| | YDWLKGKQFTCKVNNKALPSPIERTISKARGQAHQPSVYVL | Gln83Tyr |
| | PPSREELSKNTVSLTCLIKDFFPPDIDVEWQSNGQQEPESK | Asn208His |
| | YRTTPPQLDEDGSYFLYSKLSVDKSRWQRGDTFICAVMHEA | |
| | LHHHYTQESLSHSPGK | |
| 42 | CPAPEMLGGPSVFIFPPKPKDTLLIARTPEVTCVVVDLDPE | Exemplary variant canine |
| | DPEVQISWFVDGKQMQTAKTQPREEQFNGTYRVVSVLPIGH | IgG-B Fc (0YW) |
| | YDWLKGKQFTCKVNNKALPSPIERTISKARGQAHQPSVYVL | Gln83Tyr |
| | PPSREELSKNTVSLTCLIKDFFPPDIDVEWQSNGQQEPESK | Asn208Trp |
| | YRTTPPQLDEDGSYFLYSKLSVDKSRWQRGDTFICAVMHEA | |
| | LHWHYTQESLSHSPGK | |
| 43 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (YYR) |
| | YDWLKGKQFTCKVNNKALPSPIERTISKARGQAHQPSVYVL | Leu24Tyr |
| | PPSREELSKNTVSLTCLIKDFFPPDIDVEWQSNGQQEPESK | Gln83Tyr |
| | YRTTPPQLDEDGSYFLYSKLSVDKSRWQRGDTFICAVMHEA | Asn208Arg |
| | LHRHYTQESLSHSPGK | |
| 44 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (YYF) |
| | YDWLKGKQFTCKVNNKALPSPIERTISKARGQAHQPSVYVL | Leu24Tyr |
| | PPSREELSKNTVSLTCLIKDFFPPDIDVEWQSNGQQEPESK | Gln83Tyr |
| | YRTTPPQLDEDGSYFLYSKLSVDKSRWQRGDTFICAVMHEA | Asn208Phe |
| | LHFHYTQESLSHSPGK | |
| 45 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (YYY) |
| | YDWLKGKQFTCKVNNKALPSPIERTISKARGQAHQPSVYVL | Leu24Tyr |
| | PPSREELSKNTVSLTCLIKDFFPPDIDVEWQSNGQQEPESK | Gln83Tyr |
| | YRTTPPQLDEDGSYFLYSKLSVDKSRWQRGDTFICAVMHEA | Asn208Tyr |
| | LHYHYTQESLSHSPGK | |
| 46 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (YYH) |
| | YDWLKGKQFTCKVNNKALPSPIERTISKARGQAHQPSVYVL | Leu24Tyr |
| | PPSREELSKNTVSLTCLIKDFFPPDIDVEWQSNGQQEPESK | Gln83Tyr |
| | YRTTPPQLDEDGSYFLYSKLSVDKSRWQRGDTFICAVMHEA | Asn208His |
| | LHHHYTQESLSHSPGK | |
| 47 | CPAPEMLGGPSVFIFPPKPKDTLYIARTPEVTCVVVDLDPE | Exemplary variant canine |
| | DPEVQISWFVDGKQMQTAKTQPREEQFNGTYRVVSVLPIGH | IgG-B Fc (YYW) |
| | YDWLKGKQFTCKVNNKALPSPIERTISKARGQAHQPSVYVL | Leu24Tyr |
| | PPSREELSKNTVSLTCLIKDFFPPDIDVEWQSNGQQEPESK | Gln83Tyr |
| | YRTTPPQLDEDGSYFLYSKLSVDKSRWQRGDTFICAVMHEA | Asn208Trp |
| | LHWHYTQESLSHSPGK | |
| 48 | CPAPEMLGGPSVFIFPPKPKDTLYITREPEVTCVVVDLDPE | Exemplary variant canine |
| | DPEVQISWFVDGKQMQTAKTQPREEQFNGTYRVVSVLPIGH | IgG-B Fc (YTE) |
| | QDWLKGKQFTCKVNNKALPSPIERTISKARGQAHQPSVYVL | Leu24Tyr |
| | PPSREELSKNTVSLTCLIKDFFPPDIDVEWQSNGQQEPESK | Ala26Thr |
| | YRTTPPQLDEDGSYFLYSKLSVDKSRWQRGDTFICAVMHEA | Thr28Glu |
| | LHNHYTQESLSHSPGK | |
| 49 | EVQLVESGPSLVKPGGSLRLTCSVTGDSITSGYWNWIRKFP | Exemplary caninized |
| | GNKLEYMGYISYSGITDYNPSLKSRITISRDTSKNQYYLQL | variable heavy chain of |
| | NSVTTEDTATYYCARYGNYGYAMDYWGQGTLVTVSSASTTA | mouse anti-canine IL31 |
| | PSVFPLAPSCGSTSGSTVALACLVSGYFPEPVTVSWNSGSL | clone M14 and canine |
| | TSGVHTFPSVLQSSGLYSLSSMVTVPSSRWPSETFTCNVAH | IgG-B |
| | PASKTKVDKPVPKRENGRVPRPPDCPKCPAPEMLGGPSVFI | |
| | FPPKPKDTLLIARTPEVTCVVVDLDPEDPEVQISWFVDGKQ | |
| | MQTAKTQPREEQFNGTYRVVSVLPIGHQDWLKGKQFTCKVN | |
| | NKALPSPIERTISKARGQAHQPSVYVLPPSREELSKNTVSL | |
| | TCLIKDFFPPDIDVEWQSNGQQEPESKYRTTPPQLDEDGSY | |
| | FLYSKLSVDKSRWQRGDTFICAVMHEALHNHYTQESLSHSP | |
| | GK | |
| 50 | EVQLVESGPSLVKPGGSLRLTCSVTGDSITSGYWNWIRKFP | Exemplary caninized |
| | GNKLEYMGYISYSGITDYNPSLKSRITISRDTSKNQYYLQL | variable heavy chain of |
| | NSVTTEDTATYYCARYGNYGYAMDYWGQGTLVTVSSASTTA | mouse anti-canine IL31 |
| | PSVFPLAPSCGSTSGSTVALACLVSGYFPEPVTVSWNSGSL | clone M14 and YYY |
| | TSGVHTFPSVLQSSGLYSLSSMVTVPSSRWPSETFTCNVAH | variant canine IgG-B |
| | PASKTKVDKPVPKRENGRVPRPPDCPKCPAPEMLGGPSVFI | Leu24Tyr |
| | FPPKPKDTLYIARTPEVTCVVVDLDPEDPEVQISWFVDGKQ | Gln83Tyr |
| | MQTAKTQPREEQFNGTYRVVSVLPIGHYDWLKGKQFTCKVN | Asn208Tyr |
| | NKALPSPIERTISKARGQAHQPSVYVLPPSREELSKNTVSL | (amino acid substitution |
| | TCLIKDFFPPDIDVEWQSNGQQEPESKYRTTPPQLDEDGSY | numbering corresponding |
| | FLYSKLSVDKSRWQRGDTFICAVMHEALHYHYTQESLSHSP | to position of canine |
| | GK | IgG-B Fc (SEQ ID NO: 2)) |
| 51 | EVQLVESGPSLVKPGGSLRLTCSVTGDSITSGYWNWIRKFP | Exemplary caninized |
| | GNKLEYMGYISYSGITDYNPSLKSRITISRDTSKNQYYLQL | variable heavy chain of |
| | NSVTTEDTATYYCARYGNYGYAMDYWGQGTLVTVSSASTTA | mouse anti-canine IL31 |
| | PSVFPLAPSCGSTSGSTVALACLVSGYFPEPVTVSWNSGSL | clone M14 and Y0Y |
| | TSGVHTFPSVLQSSGLYSLSSMVTVPSSRWPSETFTCNVAH | variant canine IgG-B |
| | PASKTKVDKPVPKRENGRVPRPPDCPKCPAPEMLGGPSVFI | Leu24Tyr |
| | FPPKPKDTLYIARTPEVTCVVVDLDPEDPEVQISWFVDGKQ | Asn208Tyr |
| | MQTAKTQPREEQFNGTYRVVSVLPIGHQDWLKGKQFTCKVN | (amino acid substitution |
| | NKALPSPIERTISKARGQAHQPSVYVLPPSREELSKNTVSL | numbering corresponding |
| | TCLIKDFFPPDIDVEWQSNGQQEPESKYRTTPPQLDEDGSY | to position of canine |
| | FLYSKLSVDKSRWQRGDTFICAVMHEALHYHYTQESLSHSP | IgG-B Fc (SEQ ID NO: 2)) |
| | GK | |
| 52 | EVQLVESGPSLVKPGGSLRLTCSVTGDSITSGYWNWIRKFP | Exemplary caninized |
| | GNKLEYMGYISYSGITDYNPSLKSRITISRDTSKNQYYLQL | variable heavy chain of |
| | NSVTTEDTATYYCARYGNYGYAMDYWGQGTLVTVSSASTTA | mouse anti-canine IL31 |
| | PSVFPLAPSCGSTSGSTVALACLVSGYFPEPVTVSWNSGSL | clone M14 and YTE |
| | TSGVHTFPSVLQSSGLYSLSSMVTVPSSRWPSETFTCNVAH | variant canine IgG-B |
| | PASKTKVDKPVPKRENGRVPRPPDCPKCPAPEMLGGPSVFI | Leu24Tyr |
| | FPPKPKDTLYITREPEVTCVVVDLDPEDPEVQISWFVDGKQ | Ala26Thr |
| | MQTAKTQPREEQFNGTYRVVSVLPIGHQDWLKGKQFTCKVN | Thr28Glu |
| | NKALPSPIERTISKARGQAHQPSVYVLPPSREELSKNTVSL | (amino acid substitution |
| | TCLIKDFFPPDIDVEWQSNGQQEPESKYRTTPPQLDEDGSY | numbering corresponding |
| | FLYSKLSVDKSRWQRGDTFICAVMHEALHNHYTQESLSHSP | to position of canine |
| | GK | IgG-B Fc (SEQ ID NO: 2)) |
| 53 | DIVMTQSPASLSVSLGQRATISCRASESVDTYGNSFMHWYQ | Exemplary caninized light |
| | QKPGQSPKLLIYRASNLESGIPARFGGSGSGTDFTLTIDPV | chain of mouse anti-canine |
| | QADDVATYYCQQSYEDPWTFGGGTKLEIKRNDAQPAVYLFQ | IL31 clone M14 and |
| | PSPDQLHTGSASVVCLLNSFYPKDINVKWKVDGVIQDTGIQ | canine kappa light chain |
| | ESVTEQDKDSTYSLSSTLTMSSTEYLSHELYSCEITHKSLP | constant region |
| | STLIKSFQRSECQRVD | |
| 18 | MGVPRPRSWGLGFLLFLLPTLRAADSHLSLLYHLTAVSAPP | Exemplary canine FcRn |
| | PGTPAFWASGWLGPQQYLSYNNLRAQAEPYGAWVWENQVSW | with poly-His |
| | YWEKETTDLRTKEGLFLEALKALGDGGPYTLQGLLGCELGP | |
| | DNTSVPVAKFALNGEDFMTFDPKLGTWNGDWPETETVSKRW | |
| | MQQAGAVSKERTFLLYSCPQRLLGHLERGRGNLEWKEPPSM | |
| | RLKARPGSPGFSVLTCSAFSFYPPELQLRFLRNGLAAGSGE | |
| | GDFGPNGDGSFHAWSSLTVKSGDEHHYRCLVQHAGLPQPLT | |
| | VELESPAKSSGSHHHHHH | |
| 19 | HLSLLYHLTAVSAPPPGTPAFWASGWLGPQQYLSYNNLRAQ | Exemplary canine FcRn |
| | AEPYGAWVWENQVSWYWEKETTDLRTKEGLFLEALKALGDG | ECD |
| | GPYTLQGLLGCELGPDNTSVPVAKFALNGEDFMTFDPKLGT | |
| | WNGDWPETETVSKRWMQQAGAVSKERTFLLYSCPQRLLGHL | |
| | ERGRGNLEWKEPPSMRLKARPGSPGFSVLTCSAFSFYPPEL | |
| | QLRFLRNGLAAGSGEGDFGPNGDGSFHAWSSLTVKSGDEHH | |
| | YRCLVQHAGLPQPLTVELESPAK | |
| 20 | MAPRPALATAGFLALLLILLAACRLDAVQHPPKIQVYSRHP | Exemplary canine B2M |
| | AENGKPNFLNCYVSGFHPPEIEIDLLKNGKEMKAEQTDLSF | |
| | SKDWTFYLLVHTEFTPNEQDEFSCRVKHVTLSEPQIVKWDR | |
| | DN | |
| 21 | MGVPRPQPWGLGFLLFLLPTLRAAESHLSLLYHLTAVSSPA | Exemplary feline FcRn |
| | PGTPAFWVSGWLGPQQYLSYNNMRAQAEPCGAWVWENQVSW | with poly-His |
| | YWEKETTDLRNKQELFLEALKVLGEGGPYTLQGLLGCELGP | |
| | DNASVPVAKFALNGEDFMDFDPKLGTWSGEWPETETISKRW | |
| | MQEAGAVSKERTFLLNSCPQRLLGHLERGRGNLEWKEPPSM | |
| | RLKARPGSPGFSVLTCSAFSFYPPELQLRFLRNGLAAGSGE | |
| | GDFGPNGDGSFHAWSSLTVKSGDEHHYRCLVQHAGLPQPLT | |
| | VELESPAKSSGSHHHHHH | |
| 22 | HLSLLYHLTAVSSPAPGTPAFWVSGWLGPQQYLSYNNMRAQ | Exemplary feline |
| | AEPCGAWVWENQVSWYWEKETTDLRNKQELFLEALKVLGEG | FcRn ECD |
| | GPYTLQGLLGCELGPDNASVPVAKFALNGEDFMDFDPKLGT | |
| | WSGEWPETETISKRWMQEAGAVSKERTFLLNSCPQRLLGHL | |
| | ERGRGNLEWKEPPSMRLKARPGSPGFSVLTCSAFSFYPPEL | |
| | QLRFLRNGLAAGSGEGDFGPNGDGSFHAWSSLTVKSGDEHH | |
| | YRCLVQHAGLPQPLTVELESPAK | |
| 23 | MARFVVLVLLGLLYLSHLDAVQHSPKVQVYSRHPAENGKPN | Exemplary feline |
| | FLNCYVSGFHPPQIDITLMKNGKKMEAEQTDLSFNRDWTFY | B2M |
| | LLVHTEFTPTVEDEYSCQVNHTTLSEPKVVKWDRDM | |
| 24 | PPDSGSVCLTLGLPLPLWICPLVCGSVLLCLGRSPPPPQVC | Exemplary equine |
| | PVSLGLSLLSLSFCLSGSPSSPHPPPALFTRFSPAESRPSL | FcRn |
| | LYHFTAVSSPAPGAPAFWVSGWLGPQQYLSYNNLRAQAEPC | |
| | GAWVWENQVSWYWEKETTDLRKKEKLFLDAFQVLTEEGPHT | |
| | LQGLLGCELNPDNTSVPVAKFALDGEDFMEFDLKLGAWNGD | |
| | WPEALAIGQRWARQADTTKMCRSSLLYSCPERLLRERERRR | |
| | ENLEWKEPPSMRLKARPGSPGFSVLTCSAFSFYPPELQLRF | |
| | LRNGLAAGSGEGDLGPNGDGSFYAWSSLTVKSGDEYHYRCW | |
| | VQHAGLAQPLTVELESPAKSPMQVVGIVVGVLLLLVLAAGG | |
| | ALLWWRMRKGLPAPWISLRGDDIGALLPAPGLPKDADS | |
| 25 | LLYHFTAVSSPAPGAPAFWVSGWLGPQQYLSYNNLRAQAEP | Exemplary equine |
| | CGAWVWENQVSWYWEKETTDLRKKEKLFLDAFQVLTEEGPH | FcRn ECD |
| | TLQGLLGCELNPDNTSVPVAKFALDGEDFMEFDLKLGAWNG | |
| | DWPEALAIGQRWARQADTTKMCRSSLLYSCPERLLRERERR | |
| | RENLEWKEPPSMRLKARPGSPGFSVLTCSAFSFYPPELQLR | |
| | FLRNGLAAGSGEGDLGPNGDGSFYAWSSLTVKSGDEYHYRC | |
| | WVQHAGLAQPLTVELESPAK | |
| 26 | MARVVALVLLGLLSLTGLEAVPRVPKVQVYSRHPAENGKPN | Exemplary equine |
| | FLNCYVSGFHPPEIEIDLLKNGEKMKVDRSDLSFSKDWSFY | B2M |
| | LLVHTDFTPNGVDEYSCRVQHSTLKDPLIVKWDRDL | |
| 55 | CPVPEPLGGPSVLIFPPKPKDILFITRTPEVTCVVLDLGRE | Exemplary variant canine |
| | DPEVQISWFVDGKEVHTAKTQSREQQFNGTYRVVSVLPIEH | IgG-A Fc (F00) |
| | QDWLTGKEFKCRVNHIDLPSPIERTISKARGRAHKPSVYVL | Arg24Phe |
| | PPSPKELSSSDTVSITCLIKDFYPPDIDVEWQSNGQQEPER | |
| | KHRMTPPQLDEDGSYFLYSKLSVDKSRWQQGDPFTCAVMHE | |
| | TLQNHYTDLSLSHSPGK | |
| 56 | CPGCGLLGGPSVFIFPPKPKDILFTARTPTVTCVVVDLDPE | Exemplary variant canine |
| | NPEVQISWFVDSKQVQTANTQPREEQSNGTYRVVSVLPIGH | IgG-C Fc (F00) |
| | QDWLSGKQFKCKVNNKALPSPIEEIISKTPGQAHQPNVYVL | Val24Phe |
| | PPSRDEMSKNTVTLTCLVKDFFPPEIDVEWQSNGQQEPESK | |
| | YRMTPPQLDEDGSYFLYSKLSVDKSRWQRGDTFICAVMHEA | |
| | LHNHYTQISLSHSPGK | |
| 57 | CPVPESLGGPSVFIFPPKPKDILFITRTPEITCVVLDLGRE | Exemplary variant canine |
| | DPEVQISWFVDGKEVHTAKTQPREQQFNSTYRVVSVLPIEH | IgG-D Fc (F00) |
| | QDWLTGKEFKCRVNHIGLPSPIERTISKARGQAHQPSVYVL | Arg24Phe |
| | PPSPKELSSSDTVTLTCLIKDFFPPEIDVEWQSNGQPEPES | |
| | KYHTTAPQLDEDGSYFLYSKLSVDKSRWQQGDTFTCAVMHE | |
| | PGKALQNHYTDLSLSHS | |
| 58 | CPVPEPLGGPSVLIFPPKPKDILYITRTPEVTCVVLDLGRE | Exemplary variant canine |
| | DPEVQISWFVDGKEVHTAKTQSREQQFNGTYRVVSVLPIEH | IgG-A Fc (Y00) |
| | QDWLTGKEFKCRVNHIDLPSPIERTISKARGRAHKPSVYVL | Arg24Tyr |
| | PPSPKELSSSDTVSITCLIKDFYPPDIDVEWQSNGQQEPER | |
| | KHRMTPPQLDEDGSYFLYSKLSVDKSRWQQGDPFTCAVMHE | |
| | TLQNHYTDLSLSHSPGK | |
| 59 | CPGCGLLGGPSVFIFPPKPKDILYTARTPTVTCVVVDLDPE | Exemplary variant canine |
| | NPEVQISWFVDSKQVQTANTQPREEQSNGTYRVVSVLPIGH | IgG-C Fc (Y00) |
| | QDWLSGKQFKCKVNNKALPSPIEEIISKTPGQAHQPNVYVL | Val24Tyr |
| | PPSRDEMSKNTVTLTCLVKDFFPPEIDVEWQSNGQQEPESK | |
| | YRMTPPQLDEDGSYFLYSKLSVDKSRWQRGDTFICAVMHEA | |
| | LHNHYTQISLSHSPGK | |
| 60 | CPVPESLGGPSVFIFPPKPKDILYITRTPEITCVVLDLGRE | Exemplary variant canine |
| | DPEVQISWFVDGKEVHTAKTQPREQQFNSTYRVVSVLPIEH | IgG-D Fc (Y00) |
| | QDWLTGKEFKCRVNHIGLPSPIERTISKARGQAHQPSVYVL | Arg24Tyr |
| | PPSPKELSSSDTVTLTCLIKDFFPPEIDVEWQSNGQPEPES | |
| | KYHTTAPQLDEDGSYFLYSKLSVDKSRWQQGDTFTCAVMHE | |
| | ALQNHYTDLSLSHSPGK | |
| 61 | CPAPELLGGPSVFIFPPNPKDTLFITRTPEVTCVVVDVSQE | Exemplary variant equine |
| | NPDVKFNWYMDGVEVRTATTRPKEEQFNSTYRVVSVLRIQH | IgG1 Fc (F00) |
| | QDWLSGKEFKCKVNNQALPQPIERTITKTKGRSQEPQVYVL | Met24Phe |
| | APHPDELSKSKVSVTCLVKDFYPPEINIEWQSNGQPELETK | |
| | YSTTQAQQDSDGSYFLYSKLSVDRNRWQQGTTFTCGVMHEA | |
| | LHNHYTQKNVSKNPGK | |
| 62 | YTHSKFLGGPSVFIFPPNPKDALFISRTPVVTCVVVNLSDQ | Exemplary variant equine |
| | YPDVQFSWYVDNTEVHSAITKQREAQFNSTYRVVSVLPIQH | IgG2 Fc (F00) |
| | QDWLSGKEFKCSVTNVGVPQPISRAISRGKGPSRVPQVYVL | Met24Phe |
| | PPHPDELAKSKVSVTCLVKDFYPPDISVEWQSNRWPELEGK | |
| | YSTTPAQLDGDGSYFLYSKLSLETSRWQQVESFTCAVMHEA | |
| | LHNHFTKTDISESLGK | |
| 63 | CPAPELLGGPSVFIFPPKPKDVLFITRTPEVTCLVVDVSHD | Exemplary variant equine |
| | SSDVLFTWYVDGTEVKTAKTMPNEEQNNSTYRVVSVLRIQH | IgG3 Fc (F00) |
| | QDWLNGKKFKCKVNNQALPAPVERTISKATGQTRVPQVYVL | Met24Phe |
| | APHPDELSKNKVSVTCLVKDFLPTDITVEWQSNEHPEPEGK | |
| | YRTTEAQKDSDGSYFLYSKLTVETDRWQQGTTFTCVVMHEA | |
| | LHNHVMQKNVSHSPGK | |
| 64 | CPAECLQVGPSVFIFPPKPKDVLFISRTPTVTCVVVDVGHD | Exemplary variant equine |
| | FPDVQFNWYVDGVETHTATTEPKQEQFNSTYRVVSVLPIQH | IgG4 Fc (F00) |
| | KDWLSGKEFKCKVNNKALPAPVERTISKPTGQPREPQVYVL | Met24Phe |
| | APHRDELSKNKVSVTCLVKDFYPTDIDIEWKSNGQPEPETK | |
| | YSTTPAQLDSDGSYFLYSKLTVETNRWQQGTTFTCAVMHEA | |
| | LHNHYTEKSVSKSPGK | |
| 65 | CPAPELPGGPSVFIFPPKPKDVLFISRKPEVTCVVVDLGHD | Exemplary variant equine |
| | DPDVQFTWFVDGVETHTATTEPKEEQFNSTYRVVSVLPIQH | IgG5 Fc (F00) |
| | QDWLSGKEFKCSVTNKALPAPVERTTSKAKGQLRVPQVYVL | Lys24Phe |
| | APHPDELAKNTVSVTCLVKDFYPPEIDVEWQSNEHPEPEGK | |
| | YSTTPAQLNSDGSYFLYSKLSVETSRWKQGESFTCGVMHEA | |
| | VENHYTQKNVSHSPGK | |
| 66 | CCCPKCPGRPSVFIFPPNPKDTLFISRTPEVTCVVVDVSQE | Exemplary variant equine |
| | NPDVKFNWYVDGVEAHTATTKAKEKQDNSTYRVVSVLPIQH | IgG6 Fc (F00) |
| | QDWRRGKEFKCKVNNRALPAPVERTITKAKGELQDPKVYIL | Met24Phe |
| | APHREEVTKNTVSVTCLVKDFYPPDINVEWQSNEEPEPEVK | |
| | YSTTPAQLDGDGSYFLYSKLTVETDRWEQGESFTCVVMHEA | |
| | IRHTYRQKSITNFPGK | |
| 67 | TCPECLSVGPSVFIFPPKPKDVLFISRTPTVTCVVVDVGHD | Exemplary variant equine |
| | FPDVQFNWYVDGVETHTATTEPKQEQNNSTYRVVSILAIQH | IgG7 Fc (F00) |
| | KDWLSGKEFKCKVNNQALPAPVQKTISKPTGQPREPQVYVL | Met24Phe |
| | APHRDELSKNKVSVTCLVKDFYPTDIDIEWKSNGQPEPETK | |
| | YSTTPAQLDSDGSYFLYSKLTVETNRWQQGTTFTCAVMHEA | |
| | LHNHYTEKSVSKSPGK | |
| 68 | CPAPELLGGPSVFIFPPNPKDTLYITRTPEVTCVVVDVSQE | Exemplary variant equine |
| | NPDVKFNWYMDGVEVRTATTRPKEEQFNSTYRVVSVLRIQH | IgG1 Fc (Y00) |
| | QDWLSGKEFKCKVNNQALPQPIERTITKTKGRSQEPQVYVL | Met24Tyr |
| | APHPDELSKSKVSVTCLVKDFYPPEINIEWQSNGQPELETK | |
| | YSTTQAQQDSDGSYFLYSKLSVDRNRWQQGTTFTCGVMHEA | |
| | LHNHYTQKNVSKNPGK | |
| 69 | YTHSKFLGGPSVFIFPPNPKDALYISRTPVVTCVVVNLSDQ | Exemplary variant equine |
| | YPDVQFSWYVDNTEVHSAITKQREAQFNSTYRVVSVLPIQH | IgG2 Fc (Y00) |
| | QDWLSGKEFKCSVTNVGVPQPISRAISRGKGPSRVPQVYVL | Met24Tyr |
| | PPHPDELAKSKVSVTCLVKDFYPPDISVEWQSNRWPELEGK | |
| | YSTTPAQLDGDGSYFLYSKLSLETSRWQQVESFTCAVMHEA | |
| | LHNHFTKTDISESLGK | |
| 70 | CPAPELLGGPSVFIFPPKPKDVLYITRTPEVTCLVVDVSHD | Exemplary variant equine |
| | SSDVLFTWYVDGTEVKTAKTMPNEEQNNSTYRVVSVLRIQH | IgG3 Fc (Y00) |
| | QDWLNGKKFKCKVNNQALPAPVERTISKATGQTRVPQVYVL | Met24Tyr |
| | APHPDELSKNKVSVTCLVKDFLPTDITVEWQSNEHPEPEGK | |
| | YRTTEAQKDSDGSYFLYSKLTVETDRWQQGTTFTCVVMHEA | |
| | LHNHVMQKNVSHSPGK | |
| 71 | CPAECLQVGPSVFIFPPKPKDVLYISRTPTVTCVVVDVGHD | Exemplary variant equine |
| | FPDVQFNWYVDGVETHTATTEPKQEQFNSTYRVVSVLPIQH | IgG4 Fc (Y00) |
| | KDWLSGKEFKCKVNNKALPAPVERTISKPTGQPREPQVYVL | Met24Tyr |
| | APHRDELSKNKVSVTCLVKDFYPTDIDIEWKSNGQPEPETK | |
| | YSTTPAQLDSDGSYFLYSKLTVETNRWQQGTTFTCAVMHEA | |
| | LHNHYTEKSVSKSPGK | |
| 72 | CPAPELPGGPSVFIFPPKPKDVLYISRKPEVTCVVVDLGHD | Exemplary variant equine |
| | DPDVQFTWFVDGVETHTATTEPKEEQFNSTYRVVSVLPIQH | IgG5 Fc (Y00) |
| | QDWLSGKEFKCSVTNKALPAPVERTTSKAKGQLRVPQVYVL | Lys24Tyr |
| | APHPDELAKNTVSVTCLVKDFYPPEIDVEWQSNEHPEPEGK | |
| | YSTTPAQLNSDGSYFLYSKLSVETSRWKQGESFTCGVMHEA | |
| | VENHYTQKNVSHSPGK | |
| 73 | CCCPKCPGRPSVFIFPPNPKDTLYISRTPEVTCVVVDVSQE | Exemplary variant equine |
| | NPDVKFNWYVDGVEAHTATTKAKEKQDNSTYRVVSVLPIQH | IgG6 Fc (Y00) |
| | QDWRRGKEFKCKVNNRALPAPVERTITKAKGELQDPKVYIL | Met24Tyr |
| | APHREEVTKNTVSVTCLVKDFYPPDINVEWQSNEEPEPEVK | |
| | YSTTPAQLDGDGSYFLYSKLTVETDRWEQGESFTCVVMHEA | |
| | IRHTYRQKSITNFPGK | |
| 74 | TCPECLSVGPSVFIFPPKPKDVLYISRTPTVTCVVVDVGHD | Exemplary variant equine |
| | FPDVQFNWYVDGVETHTATTEPKQEQNNSTYRVVSILAIQH | IgG7 Fc (Y00) |
| | KDWLSGKEFKCKVNNQALPAPVQKTISKPTGQPREPQVYVL | Met24Tyr |
| | APHRDELSKNKVSVTCLVKDFYPTDIDIEWKSNGQPEPETK | |
| | YSTTPAQLDSDGSYFLYSKLTVETNRWQQGTTFTCAVMHEA | |
| | LHNHYTEKSVSKSPGK | |
| 75 | CPPPEMLGGPSIFIFPPKPKDTLFISRTPEVTCLVVDLGPD | Exemplary variant feline |
| | DSDVQITWFVDNTQVYTAKTSPREEQFNSTYRVVSVLPILH | IgG1a Fc (F00) |
| | QDWLKGKEFKCKVNSKSLPSPIERTISKDKGQPHEPQVYVL | Ser24Phe |
| | PPAQEELSRNKVSVTCLIEGFYPSDIAVEWEITGQPEPENN | |
| | YRTTPPQLDSDGTYFLYSRLSVDRSRWQRGNTYTCSVSHEA | |
| | LHSHHTQKSLTQSPGK | |
| 76 | CPPPEMLGGPSIFIFPPKPKDTLFISRTPEVTCLVVDLGPD | Exemplary variant feline |
| | DSDVQITWFVDNTQVYTAKTSPREEQFNSTYRVVSVLPILH | IgG1b Fc (F00) |
| | QDWLKGKEFKCKVNSKSLPSPIERTISKAKGQPHEPQVYVL | Ser24Phe |
| | PPAQEELSRNKVSVTCLIKSFHPPDIAVEWEITGQPEPENN | |
| | YRTTPPQLDSDGTYFVYSKLSVDRSHWQRGNTYTCSVSHEA | |
| | LHSHHTQKSLTQSPGK | |
| 77 | CPVPEIPGAPSVFIFPPKPKDTLFISRTPEVTCLVVDLGPD | Exemplary variant feline |
| | DSNVQITWFVDNTEMHTAKTRPREEQFNSTYRVVSVLPILH | IgG2 Fc (F00) |
| | QDWLKGKEFKCKVNSKSLPSAMERTISKAKGQPHEPQVYVL | Ser24Phe |
| | PPTQEELSENKVSVTCLIKGFHPPDIAVEWEITGQPEPENN | |
| | YQTTPPQLDSDGTYFLYSRLSVDRSHWQRGNTYTCSVSHEA | |
| | LHSHHTQKSLTQSPGK | |
| 78 | CPPPEMLGGPSIFIFPPKPKDTLTISRTPEVTCLVVDLGPD | Exemplary variant feline |
| | DSDVQITWFVDNTQVYTAKTSPREEQFNSTYRVVSVLPILH | IgG1a Fc (Y00) |
| | QDWLKGKEFKCKVNSKSLPSPIERTISKDKGQPHEPQVYVL | Ser24Tyr |
| | PPAQEELSRNKVSVTCLIEGFYPSDIAVEWEITGQPEPENN | |
| | YRTTPPQLDSDGTYFLYSRLSVDRSRWQRGNTYTCSVSHEA | |
| | LHSHHTQKSLTQSPGK | |
| 79 | CPPPEMLGGPSIFIFPPKPKDTLTISRTPEVTCLVVDLGPD | Exemplary variant feline |
| | DSDVQITWFVDNTQVYTAKTSPREEQFNSTYRVVSVLPILH | IgG1a Fc (Y00) |
| | QDWLKGKEFKCKVNSKSLPSPIERTISKAKGQPHEPQVYVL | Ser24Tyr |
| | PPAQEELSRNKVSVTCLIKSFHPPDIAVEWEITGQPEPENN | |
| | YRTTPPQLDSDGTYFVYSKLSVDRSHWQRGNTYTCSVSHEA | |
| | LHSHHTQKSLTQSPGK | |
| 80 | CPVPEIPGAPSVFIFPPKPKDTLTISRTPEVTCLVVDLGPD | Exemplary variant feline |
| | DSNVQITWFVDNTEMHTAKTRPREEQFNSTYRVVSVLPILH | IgG2 Fc (Y00) |
| | QDWLKGKEFKCKVNSKSLPSAMERTISKAKGQPHEPQVYVL | Ser24Tyr |
| | PPTQEELSENKVSVTCLIKGFHPPDIAVEWEITGQPEPENN | |
| | YQTTPPQLDSDGTYFLYSRLSVDRSHWQRGNTYTCSVSHEA | |
| | LHSHHTQKSLTQSPGK | |
| 81 | CPPPEMLGGPSIFIFPPKPKDTLYITREPEVTCLVVDLGPD | Exemplary variant feline |
| | DSDVQITWFVDNTQVYTAKTSPREEQFNSTYRVVSVLPILH | IgG1a Fc (YTE) |
| | QDWLKGKEFKCKVNSKSLPSPIERTISKDKGQPHEPQVYVL | Ser24Tyr |
| | PPAQEELSRNKVSVTCLIEGFYPSDIAVEWEITGQPEPENN | Ser26Thr |
| | YRTTPPQLDSDGTYFLYSRLSVDRSRWQRGNTYTCSVSHEA | Thr28Glu |
| | LHSHHTQKSLTQSPGK | |
| 82 | CPPPEMLGGPSIFIFPPKPKDTLYITREPEVTCLVVDLGPD | Exemplary variant feline |
| | DSDVQITWFVDNTQVYTAKTSPREEQFNSTYRVVSVLPILH | IgG1b Fc (YTE) |
| | QDWLKGKEFKCKVNSKSLPSPIERTISKAKGQPHEPQVYVL | Ser24Tyr |
| | PPAQEELSRNKVSVTCLIKSFHPPDIAVEWEITGQPEPENN | Ser26Thr |
| | YRTTPPQLDSDGTYFVYSKLSVDRSHWQRGNTYTCSVSHEA | Thr28Glu |
| | LHSHHTQKSLTQSPGK | |
| 83 | CPVPEIPGAPSVFIFPPKPKDTLYITREPEVTCLVVDLGPD | Exemplary variant feline |
| | DSNVQITWFVDNTEMHTAKTRPREEQFNSTYRVVSVLPILH | IgG2 Fc (YTE) |
| | QDWLKGKEFKCKVNSKSLPSAMERTISKAKGQPHEPQVYVL | Ser24Tyr |
| | PPTQEELSENKVSVTCLIKGFHPPDIAVEWEITGQPEPENN | Ser26Thr |
| | YQTTPPQLDSDGTYFLYSRLSVDRSHWQRGNTYTCSVSHEA | Thr28Glu |
| | LHSHHTQKSLTQSPGK | |
| 84 | CPVPEPLGGPSVLIFPPKPKDTLLIARTPEVTCVVLDLGRE | Exemplary variant canine |
| | DPEVQISWFVDGKEVHTAKTQSREQQFNGTYRVVSVLPIGH | IgG-A Fc (Protein A+; |
| | QDWLTGKEFKCRVNHIDLPSPIERTISKARGRAHKPSVYVL | C1q-; CD16-) |
| | PPSPKELSSSDTVSITCLIKDFYPPDIDVEWQSNGQQEPER | I(22)T; R(24)L; T(26)A; |
| | KHRMTPPQLDEDGSYFLYSKLSVDKSRWQQGDPFTCAVMHE | E(81)G; T(206)A; |
| | ALHNHYTDLSLSHSPGK | Q(208)H |
| 85 | CPVPEPLGGPSVLIFPPKPKDTLFIARTPEVTCVVLDLGRE | Exemplary variant canine |
| | DPEVQISWFVDGKEVHTAKTQSREQQFNGTYRVVSVLPIGH | IgG-A Fc (F00; Protein |
| | QDWLTGKEFKCRVNHIDLPSPIERTISKARGRAHKPSVYVL | A+; C1q-; CD16-) |
| | PPSPKELSSSDTVSITCLIKDFYPPDIDVEWQSNGQQEPER | I(22)T; R(24)F; T(26)A; |
| | KHRMTPPQLDEDGSYFLYSKLSVDKSRWQQGDPFTCAVMHE | E(81)G; T(206)A; |
| | ALHNHYTDLSLSHSPGK | Q(208)H |
| 86 | CPAPEMLGGPSVLIFPPKPKDTLLIARTPEVTCVVVDLDPE | Exemplary variant canine |
| | DPEVQISWFVDGKEVHTAKTQSREEQFNGTYRVVSVLPIGH | IgG-A Fc (Protein A+; |
| | QDWLTGKEFKCKVNNKALPSPIERTISKARGRAHKPSVYVL | C1q+; CD16+) |
| | PPSPKELSSSDTVSITCLIKDFYPPDIDVEWQSNGQQEPER | V3A; P6M; I22T; R24L; |
| | KHRMTPPQLDEDGSYFLYSKLSVDKSRWQQGDPFTCAVMHE | T26A; L36V; G39D; |
| | ALHNHYTDLSLSHSPGK | R40P; Q66E; E81G; |
| | | R94K; H97N; I98K; |
| | | D99A; T206A; Q208H |
| 87 | CPAPEMLGGPSVLIFPPKPKDTLFIARTPEVTCVVVDLDPE | Exemplary variant canine |
| | DPEVQISWFVDGKEVHTAKTQSREEQFNGTYRVVSVLPIGH | IgG-A Fc (F00; Protein |
| | QDWLTGKEFKCKVNNKALPSPIERTISKARGRAHKPSVYVL | A+; C1q+; CD16+) |
| | PPSPKELSSSDTVSITCLIKDFYPPDIDVEWQSNGQQEPER | V3A; P6M; I22T; R24F; |
| | KHRMTPPQLDEDGSYFLYSKLSVDKSRWQQGDPFTCAVMHE | T26A; L36V; G39D; |
| | ALHNHYTDLSLSHSPGK | R40P; Q66E; E81G; |
| | | R94K; H97N; I98K; |
| | | D99A; T206A; Q208H |
| 88 | CPVPESLGGPSVFIFPPKPKDTLLIARTPEITCVVLDLGRE | Exemplary variant canine |
| | DPEVQISWFVDGKEVHTAKTQPREQQFNSTYRVVSVLPIGH | IgG-D Fc (Protein A+; |
| | QDWLTGKEFKCRVNHIGLPSPIERTISKARGQAHQPSVYVL | C1q-; CD16-) |
| | PPSPKELSSSDTVTLTCLIKDFFPPEIDVEWQSNGQPEPES | I(22)T; R(24)L; T(26)A; |
| | KYHTTAPQLDEDGSYFLYSKLSVDKSRWQQGDTFTCAVMHE | E(81)G; Q(208)H |
| | ALHNHYTDLSLSHSPGK | |
| 89 | CPVPESLGGPSVFIFPPKPKDTLFIARTPEITCVVLDLGRE | Exemplary variant canine |
| | DPEVQISWFVDGKEVHTAKTQPREQQFNSTYRVVSVLPIGH | IgG-D Fc (F00; Protein |
| | QDWLTGKEFKCRVNHIGLPSPIERTISKARGQAHQPSVYVL | A+; C1q-; CD16-) |
| | PPSPKELSSSDTVTLTCLIKDFFPPEIDVEWQSNGQPEPES | I(22)T; R(24)F; T(26)A; |
| | KYHTTAPQLDEDGSYFLYSKLSVDKSRWQQGDTFTCAVMHE | E(81)G; Q(206)A; |
| | ALHNHYTDLSLSHSPGK | Q(208)H |
| 90 | CPAPEMLGGPSVFIFPPKPKDTLLIARTPEITCVVVDLDPE | Exemplary variant canine |
| | DPEVQISWFVDGKEVHTAKTQPREEQFNSTYRVVSVLPIGH | IgG-D Fc (Protein A+; |
| | QDWLTGKEFKCKVNNKALPSPIERTISKARGQAHQPSVYVL | C1q+; CD16+) |
| | PPSPKELSSSDTVTLTCLIKDFFPPEIDVEWQSNGQPEPES | V3A; S6M; I22T; R24L; |
| | KYHTTAPQLDEDGSYFLYSKLSVDKSRWQQGDTFTCAVMHE | T26A; L36V; G39D; |
| | ALHNHYTDLSLSHSPGK | R40P; Q66E; E81G; |
| | | R94K; H97N; I98K; |
| | | G99A; Q208H |
| 91 | CPAPEMLGGPSVFIFPPKPKDTLFIARTPEITCVVVDLDPE | Exemplary variant canine |
| | DPEVQISWFVDGKEVHTAKTQPREEQFNSTYRVVSVLPIGH | IgG-D Fc (F00; Protein |
| | QDWLTGKEFKCKVNNKALPSPIERTISKARGQAHQPSVYVL | A+; C1q+; CD16+) |
| | PPSPKELSSSDTVTLTCLIKDFFPPEIDVEWQSNGQPEPES | V3A; S6M; I22T; R24L; |
| | KYHTTAPQLDEDGSYFLYSKLSVDKSRWQQGDTFTCAVMHE | T26A; L36V; G39D; |
| | ALHNHYTDLSLSHSPGK | R40P; Q66E; E81G; |
| | | R94K; H97N; I98K; |
| | | G99A; Q208H |
| 92 | METDTLLLWVLLLWVPGSTGDIQMTQSPSSLSASVGDRVTI | Exemplary chimeric D2E7 |
| | TCRASQGIRNYLAWYQQKPGKAPKLLIYAASTLQSGVPSRF | anti-TNFα variable light |
| | SGSGSGTDFTLTISSLQPEDVATYYCQRYNRAPYTFGQGTK | chain and canine kappa |
| | VEIKRNDAQPAVYLFQPSPDQLHTGSASVVCLLNSFYPKDI | constant light chain with |
| | NVKWKVDGVIQDTGIQESVTEQDKDSTYSLSSTLTMSSTEY | leader sequence |
| | LSHELYSCEITHKSLPSTLIKSFQRSECQRVD | |
| 93 | DIQMTQSPSSLSASVGDRVTITCRASQGIRNYLAWYQQKPG | Exemplary chimeric D2E7 |
| | KAPKLLIYAASTLQSGVPSRFSGSGSGTDFTLTISSLQPED | anti-TNFα variable light |
| | VATYYCQRYNRAPYTFGQGTKVEIKRNDAQPAVYLFQPSPD | chain and canine kappa |
| | QLHTGSASVVCLLNSFYPKDINVKWKVDGVIQDTGIQESVT | constant light chain |
| | EQDKDSTYSLSSTLTMSSTEYLSHELYSCEITHKSLPSTLI | without leader sequence |
| | KSFQRSECQRVD | |
| 94 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and wild-type canine |
| | ASSLDYWGQGTLVTVSSASTTAPSVFPLAPSCGSTSGSTVA | IgG-B Fc with leader |
| | LACLVSGYFPEPVTVSWNSGSLTSGVHTFPSVLQSSGLYSL | sequence |
| | SSMVTVPSSRWPSETFTCNVAHPASKTKVDKPVPKRENGRV | |
| | PRPPDCPKCPAPEMLGGPSVFIFPPKPKDTLLIARTPEVTC | |
| | VVVDLDPEDPEVQISWFVDGKQMQTAKTQPREEQFNGTYRV | |
| | VSVLPIGHQDWLKGKQFTCKVNNKALPSPIERTISKARGQA | |
| | HQPSVYVLPPSREELSKNTVSLTCLIKDFFPPDIDVEWQSN | |
| | GQQEPESKYRTTPPQLDEDGSYFLYSKLSVDKSRWQRGDTF | |
| | ICAVMHEALHNHYTQESLSHSPGK | |
| 95 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and wild-type canine |
| | TTAPSVFPLAPSCGSTSGSTVALACLVSGYFPEPVTVSWNS | IgG-B Fc without leader |
| | GSLTSGVHTFPSVLQSSGLYSLSSMVTVPSSRWPSETFTCN | sequence |
| | VAHPASKTKVDKPVPKRENGRVPRPPDCPKCPAPEMLGGPS | |
| | VFIFPPKPKDTLLIARTPEVTCVVVDLDPEDPEVQISWFVD | |
| | GKQMQTAKTQPREEQFNGTYRVVSVLPIGHQDWLKGKQFTC | |
| | KVNNKALPSPIERTISKARGQAHQPSVYVLPPSREELSKNT | |
| | VSLTCLIKDFFPPDIDVEWQSNGQQEPESKYRTTPPQLDED | |
| | GSYFLYSKLSVDKSRWQRGDTFICAVMHEALHNHYTQESLS | |
| | HSPGK | |
| 96 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant canine |
| | ASSLDYWGQGTLVTVSSASTTAPSVFPLAPSCGSTSGSTVA | IgG-B Fc (0Y0) with |
| | LACLVSGYFPEPVTVSWNSGSLTSGVHTFPSVLQSSGLYSL | leader sequence |
| | SSMVTVPSSRWPSETFTCNVAHPASKTKVDKPVPKRENGRV | |
| | PRPPDCPKCPAPEMLGGPSVFIFPPKPKDTLLIARTPEVTC | |
| | VVVDLDPEDPEVQISWFVDGKQMQTAKTQPREEQFNGTYRV | |
| | VSVLPIGHYDWLKGKQFTCKVNNKALPSPIERTISKARGQA | |
| | HQPSVYVLPPSREELSKNTVSLTCLIKDFFPPDIDVEWQSN | |
| | GQQEPESKYRTTPPQLDEDGSYFLYSKLSVDKSRWQRGDTF | |
| | ICAVMHEALHNHYTQESLSHSPGK | |
| 97 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant canine |
| | TTAPSVFPLAPSCGSTSGSTVALACLVSGYFPEPVTVSWNS | IgG-B Fc (0Y0) without |
| | GSLTSGVHTFPSVLQSSGLYSLSSMVTVPSSRWPSETFTCN | leader sequence |
| | VAHPASKTKVDKPVPKRENGRVPRPPDCPKCPAPEMLGGPS | |
| | VFIFPPKPKDTLLIARTPEVTCVVVDLDPEDPEVQISWFVD | |
| | GKQMQTAKTQPREEQFNGTYRVVSVLPIGHYDWLKGKQFTC | |
| | KVNNKALPSPIERTISKARGQAHQPSVYVLPPSREELSKNT | |
| | VSLTCLIKDFFPPDIDVEWQSNGQQEPESKYRTTPPQLDED | |
| | GSYFLYSKLSVDKSRWQRGDTFICAVMHEALHNHYTQESLS | |
| | HSPGK | |
| 98 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant canine |
| | ASSLDYWGQGTLVTVSSASTTAPSVFPLAPSCGSTSGSTVA | IgG-B Fc (0YH) with |
| | LACLVSGYFPEPVTVSWNSGSLTSGVHTFPSVLQSSGLYSL | leader sequence |
| | SSMVTVPSSRWPSETFTCNVAHPASKTKVDKPVPKRENGRV | |
| | PRPPDCPKCPAPEMLGGPSVFIFPPKPKDTLLIARTPEVTC | |
| | VVVDLDPEDPEVQISWFVDGKQMQTAKTQPREEQFNGTYRV | |
| | VSVLPIGHYDWLKGKQFTCKVNNKALPSPIERTISKARGQA | |
| | HQPSVYVLPPSREELSKNTVSLTCLIKDFFPPDIDVEWQSN | |
| | GQQEPESKYRTTPPQLDEDGSYFLYSKLSVDKSRWQRGDTF | |
| | ICAVMHEALHHHYTQESLSHSPGK | |
| 99 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant canine |
| | TTAPSVFPLAPSCGSTSGSTVALACLVSGYFPEPVTVSWNS | IgG-B Fc (0YH) without |
| | GSLTSGVHTFPSVLQSSGLYSLSSMVTVPSSRWPSETFTCN | leader sequence |
| | VAHPASKTKVDKPVPKRENGRVPRPPDCPKCPAPEMLGGPS | |
| | VFIFPPKPKDTLLIARTPEVTCVVVDLDPEDPEVQISWFVD | |
| | GKQMQTAKTQPREEQFNGTYRVVSVLPIGHYDWLKGKQFTC | |
| | KVNNKALPSPIERTISKARGQAHQPSVYVLPPSREELSKNT | |
| | VSLTCLIKDFFPPDIDVEWQSNGQQEPESKYRTTPPQLDED | |
| | GSYFLYSKLSVDKSRWQRGDTFICAVMHEALHHHYTQESLS | |
| | HSPGK | |
| 100 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant canine |
| | ASSLDYWGQGTLVTVSSASTTAPSVFPLAPSCGSTSGSTVA | IgG-B Fc (0YY) with |
| | LACLVSGYFPEPVTVSWNSGSLTSGVHTFPSVLQSSGLYSL | leader sequence |
| | SSMVTVPSSRWPSETFTCNVAHPASKTKVDKPVPKRENGRV | |
| | PRPPDCPKCPAPEMLGGPSVFIFPPKPKDTLLIARTPEVTC | |
| | VVVDLDPEDPEVQISWFVDGKQMQTAKTQPREEQFNGTYRV | |
| | VSVLPIGHYDWLKGKQFTCKVNNKALPSPIERTISKARGQA | |
| | HQPSVYVLPPSREELSKNTVSLTCLIKDFFPPDIDVEWQSN | |
| | GQQEPESKYRTTPPQLDEDGSYFLYSKLSVDKSRWQRGDTF | |
| | ICAVMHEALHYHYTQESLSHSPGK | |
| 101 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant canine |
| | TTAPSVFPLAPSCGSTSGSTVALACLVSGYFPEPVTVSWNS | IgG-B Fc (0YY) without |
| | GSLTSGVHTFPSVLQSSGLYSLSSMVTVPSSRWPSETFTCN | leader sequence |
| | VAHPASKTKVDKPVPKRENGRVPRPPDCPKCPAPEMLGGPS | |
| | VFIFPPKPKDTLLIARTPEVTCVVVDLDPEDPEVQISWFVD | |
| | GKQMQTAKTQPREEQFNGTYRVVSVLPIGHYDWLKGKQFTC | |
| | KVNNKALPSPIERTISKARGQAHQPSVYVLPPSREELSKNT | |
| | VSLTCLIKDFFPPDIDVEWQSNGQQEPESKYRTTPPQLDED | |
| | GSYFLYSKLSVDKSRWQRGDTFICAVMHEALHYHYTQESLS | |
| | HSPGK | |
| 102 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant canine |
| | ASSLDYWGQGTLVTVSSASTTAPSVFPLAPSCGSTSGSTVA | IgG-B Fc (00Y) with |
| | LACLVSGYFPEPVTVSWNSGSLTSGVHTFPSVLQSSGLYSL | leader sequence |
| | SSMVTVPSSRWPSETFTCNVAHPASKTKVDKPVPKRENGRV | |
| | PRPPDCPKCPAPEMLGGPSVFIFPPKPKDTLLIARTPEVTC | |
| | VVVDLDPEDPEVQISWFVDGKQMQTAKTQPREEQFNGTYRV | |
| | VSVLPIGHQDWLKGKQFTCKVNNKALPSPIERTISKARGQA | |
| | HQPSVYVLPPSREELSKNTVSLTCLIKDFFPPDIDVEWQSN | |
| | GQQEPESKYRTTPPQLDEDGSYFLYSKLSVDKSRWQRGDTF | |
| | ICAVMHEALHYHYTQESLSHSPGK | |
| 103 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant canine |
| | TTAPSVFPLAPSCGSTSGSTVALACLVSGYFPEPVTVSWNS | IgG-B Fc (00Y) without |
| | GSLTSGVHTFPSVLQSSGLYSLSSMVTVPSSRWPSETFTCN | leader sequence |
| | VAHPASKTKVDKPVPKRENGRVPRPPDCPKCPAPEMLGGPS | |
| | VFIFPPKPKDTLLIARTPEVTCVVVDLDPEDPEVQISWFVD | |
| | GKQMQTAKTQPREEQFNGTYRVVSVLPIGHQDWLKGKQFTC | |
| | KVNNKALPSPIERTISKARGQAHQPSVYVLPPSREELSKNT | |
| | VSLTCLIKDFFPPDIDVEWQSNGQQEPESKYRTTPPQLDED | |
| | GSYFLYSKLSVDKSRWQRGDTFICAVMHEALHYHYTQESLS | |
| | HSPGK | |
| 104 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant canine |
| | ASSLDYWGQGTLVTVSSASTTAPSVFPLAPSCGSTSGSTVA | IgG-B Fc (F00) with |
| | LACLVSGYFPEPVTVSWNSGSLTSGVHTFPSVLQSSGLYSL | leader sequence |
| | SSMVTVPSSRWPSETFTCNVAHPASKTKVDKPVPKRENGRV | |
| | PRPPDCPKCPAPEMLGGPSVFIFPPKPKDTLFIARTPEVTC | |
| | VVVDLDPEDPEVQISWFVDGKQMQTAKTQPREEQFNGTYRV | |
| | VSVLPIGHQDWLKGKQFTCKVNNKALPSPIERTISKARGQA | |
| | HQPSVYVLPPSREELSKNTVSLTCLIKDFFPPDIDVEWQSN | |
| | GQQEPESKYRTTPPQLDEDGSYFLYSKLSVDKSRWQRGDTF | |
| | ICAVMHEALHNHYTQESLSHSPGK | |
| 105 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant canine |
| | TTAPSVFPLAPSCGSTSGSTVALACLVSGYFPEPVTVSWNS | IgG-B Fc (F00) without |
| | GSLTSGVHTFPSVLQSSGLYSLSSMVTVPSSRWPSETFTCN | leader sequence |
| | VAHPASKTKVDKPVPKRENGRVPRPPDCPKCPAPEMLGGPS | |
| | VFIFPPKPKDTLFIARTPEVTCVVVDLDPEDPEVQISWFVD | |
| | GKQMQTAKTQPREEQFNGTYRVVSVLPIGHQDWLKGKQFTC | |
| | KVNNKALPSPIERTISKARGQAHQPSVYVLPPSREELSKNT | |
| | VSLTCLIKDFFPPDIDVEWQSNGQQEPESKYRTTPPQLDED | |
| | GSYFLYSKLSVDKSRWQRGDTFICAVMHEALHNHYTQESLS | |
| | HSPGK | |
| 106 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variable canine |
| | ASSLDYWGQGTLVTVSSASTTAPSVFPLAPSCGSTSGSTVA | IgG-A Fc (Protein A+; |
| | LACLVSGYFPEPVTVSWNSGSLTSGVHTFPSVLQSSGLHSL | C1q+; CD16+) with |
| | SSMVTVPSSRWPSETFTCNVVHPASNTKVDKPVFNECRCTD | leader sequence |
| | TPPCPAPEMLGGPSVLIFPPKPKDTLLIARTPEVTCVVVDL | |
| | DPEDPEVQISWFVDGKEVHTAKTQSREEQFNGTYRVVSVLP | |
| | IGHQDWLTGKEFKCKVNNKALPSPIERTISKARGRAHKPSV | |
| | YVLPPSPKELSSSDTVSITCLIKDFYPPDIDVEWQSNGQQE | |
| | PERKHRMTPPQLDEDGSYFLYSKLSVDKSRWQQGDPFTCAV | |
| | MHEALHNHYTDLSLSHSPGK | |
| 107 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variable canine |
| | TTAPSVFPLAPSCGSTSGSTVALACLVSGYFPEPVTVSWNS | IgG-A Fc (Protein A+; |
| | GSLTSGVGTFPSVLQSSGLHSLSSMVTVPSSRWPSETFTCN | C1q+; CD16+) without |
| | VVHPASNTKVDKPVFNECRCTDTPPCPAPEMLGGPSVLIFP | leader sequence |
| | PKPKDTLLIARTPEVTCVVVDLDPEDPEVQISWFVDGKEVH | |
| | TAKTQSREEQFNGTYRVVSVLPIGHQDWLTGKEFKCKVNNK | |
| | ALPSPIERTISKARGRAHKPSVYVLPPSPKELSSSDTVSIT | |
| | TLIKDFYPPDIDVEWQSNGQQEPERKHRMTPPQLDEDGSYF | |
| | LYSKLSVDKSRWQQGDPFTCAVMHEALHNHYTDLSLSHSPG | |
| | K | |
| 108 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variable canine |
| | TTAPSVFPLAPSCGSTSGSTVALACLVSGYFPEPVTVSWNS | IgG-A Fc (F00; Protein |
| | GSLTSGVHTFPSVLQSSGLHSLSSMVTVPSSRWPSETFTCN | A+; C1q+; CD16+) |
| | VVHPASNTKVDKPVFNECRCTDTPPCPAPEMLGGPSVLIFP | without leader sequence |
| | PKPKDTLFIARTPEVTCVVVDLDPEDPEVQISWFVDGKEVH | |
| | TAKTQSREEQFNGTYRVVSVLPIGHQDWLTGKEFKCKVNNK | |
| | ALPSPIERTISKARGRAHKPSVYVLPPSPKELSSSDTVSIT | |
| | TLIKDFYPPDIDVEWQSNGQQEPERKHRMTPPQLDEDGSYF | |
| | LYSKLSVDKSRWQQGDPFTCAVMHEALHNHYTDLSLSHSPG | |
| | K | |
| 109 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variable canine |
| | ASSLDYWGQGTLVTVSSASTTAPSVFPLAPSCGSTSGSTVA | IgG-A Fc (F00, Protein |
| | LACLVSGYFPEPVTVSWNSGSLTSGVHTFPSVLQSSGLHSL | A+; C1q-; CD16-) with |
| | SSMVTVPSSRWPSETFTCNVVHPASNTKVDKPVFNECRCTD | leader sequence |
| | TPPCPVPEPLGGPSVLIFPPKPKDTLFIARTPEVTCVVLDL | |
| | GREDPEVQISWFVDGKEVHTAKTQSREQQFNGTYRVVSVLP | |
| | IGHQDWLTGKEFKCRVNHIDLPSPIERTISKARGRAHKPSV | |
| | YVLPPSPKELSSSDTVSITCLIKDFYPPDIDVEWQSNGQQE | |
| | PERKHRMTPPQLDEDGSYFLYSKLSVDKSRWQQGDPFTCAV | |
| | MHEALHNHYTDLSLSHSPGK | |
| 110 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variable canine |
| | TTAPSVFPLAPSCGSTSGSTVALACLVSGYFPEPVTVSWNS | IgG-A Fc (F00, Protein |
| | GSLTSGVHTFPSVLQSSGLHSLSSMVTVPSSRWPSETFTCN | A+; C1q-; CD16-) |
| | VVHPASNTKVDKPVFNECRCTDTPPCPVPEPLGGPSVLIFP | without leader sequence |
| | PKPKDTLFIARTPEVTCVVLDLGREDPEVQISWFVDGKEVH | |
| | TAKTQSREQQFNGTYRVVSVLPIGHQDWLTGKEFKCRVNHI | |
| | DLPSPIERTISKARGRAHKPSVYVLPPSPKELSSSDTVSIT | |
| | CLIKDFYPPDIDVEWQSNGQQEPERKHRMTPPQLDEDGSYF | |
| | LYSKLSVDKSRWQQGDPFTCAVMHEALHNHYTDLSLSHSPG | |
| | K | |
| 111 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variable canine |
| | ASSLDYWGQGTLVTVSSASTTAPSVFPLAPSCGSTSGSTVA | IgG-D Fc (Protein A+; |
| | LACLVSGYFPEPVTVSWNSGSLTSGVHTFPSVLQSSGLYSL | C1q+; CD16+) with |
| | SSTVTVPSSRWPSETFTCNVVHPASNTKVDKPVPKESTCKC | leader sequence |
| | ISPCPAPEMLGGPSVFIFPPKPKDTLLIARTPEITCVVVDL | |
| | DPEDPEVQISWFVDGKEVHTAKTQPREEQFNSTYRVVSVLP | |
| | IGHQDWLTGKEFKCKVNNKALPSPIERTISKARGQAHQPSV | |
| | YVLPPSPKELSSSDTVTLTCLIKDFFPPEIDVEWQSNGQPE | |
| | PESKYHTTAPQLDEDGSYFLYSKLSVDKSRWQQGDTFTCAV | |
| | MHEALHNHYTDLSLSHSPGK | |
| 112 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variable canine |
| | TTAPSVFPLAPSCGSTSGSTVALACLVSGYFPEPVTVSWNS | IgG-D Fc (Protein A+; |
| | GSLTSGVHTFPSVLQSSGLYSLSSTVTVPSSRWPSETFTCN | C1q+; CD16+) without |
| | VVHPASNTKVDKPVPKESTCKCISPCPAPEMLGGPSVFIFP | leader sequence |
| | PKPKDTLLIARTPEITCVVVDLDPEDPEVQISWFVDGKEVH | |
| | TAKTQPREEQFNSTYRVVSVLPIGHQDWLTGKEFKCKVNNK | |
| | ALPSPIERTISKARGQAHQPSVYVLPPSPKELSSSDTVTLT | |
| | CLIKDFFPPEIDVEWQSNGQPEPESKYHTTAPQLDEDGSYF | |
| | LYSKLSVDKSRWQQGDTFTCAVMHEALHNHYTDLSLSHSPG | |
| | K | |
| 113 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variable canine |
| | TTAPSVFPLAPSCGSTSGSTVALACLVSGYFPEPVTVSWNS | IgG-D Fc (F00; Protein |
| | GSLTSGVHTFPSVLQSSGLYSLSSTVTVPSSRWPSETFTCN | A+; C1q+; CD16+) |
| | VVHPASNTKVDKPVPKESTCKCISPCPAPEMLGGPSVFIFP | without leader sequence |
| | PKPKDTLFIARTPEITCVVVDLDPEDPEVQISWFVDGKEVH | |
| | TAKTQPREEQFNSTYRVVSVLPIGHQDWLTGKEFKCKVNNK | |
| | ALPSPIERTISKARGQAHQPSVYVLPPSPKELSSSDTVTLT | |
| | CLIKDFFPPEIDVEWQSNGQPEPESKYHTTAPQLDEDGSYF | |
| | LYSKLSVDKSRWQQGDTFTCAVMHEALHNHYTDLSLSHSPG | |
| | K | |
| 114 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variable canine |
| | ASSLDYWGQGTLVTVSSASTTAPSVFPLAPSCGSTSGSTVA | IgG-D Fc (F00, Protein |
| | LACLVSGYFPEPVTVSWNSGSLTSGVHTFPSVLQSSGLYSL | A+; C1q-; CD16-) with |
| | SSTVTVPSSRWPSETFTCNVVHPASNTKVDKPVPKESTCKC | leader sequence |
| | ISPCPVPESLGGPSVFIFPPKPKDTLFIARTPEITCVVLDL | |
| | GREDPEVQISWFVDGKEVHTAKTQPREQQFNSTYRVVSVLP | |
| | IGHQDWLTGKEFKCRVNHIGLPSPIERTISKARGQAHQPSV | |
| | YVLPPSPKELSSSDTVTLTCLIKDFFPPEIDVEWQSNGQPE | |
| | PESKYHTTAPQLDEDGSYFLYSKLSVDKSRWQQGDTFTCAV | |
| | MHEALHNHYTDLSLSHSPGK | |
| 115 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variable canine |
| | TTAPSVFPLAPSCGSTSGSTVALACLVSGYFPEPVTVSWNS | IgG-D Fc (F00, Protein |
| | GSLTSGVHTFPSVLQSSGLYSLSSTVTVPSSRWPSETFTCN | A+; C1q-; CD16-) |
| | VVHPASNTKVDKPVPKESTCKCISPCPVPESLGGPSVFIFP | without leader sequence |
| | PKPKDTLFIARTPEITCVVLDLGREDPEVQISWFVDGKEVH | |
| | TAKTQPREQQFNSTYRVVSVLPIGHQDWLTGKEFKCRVNHI | |
| | GLPSPIERTISKARGQAHQPSVYVLPPSPKELSSSDTVTLT | |
| | CLIKDFFPPEIDVEWQSNGQPEPESKYHTTAPQLDEDGSYF | |
| | LYSKLSVDKSRWQQGDTFTCAVMHEALHNHYTDLSLSHSPG | |
| | K | |
| 116 | METDTLLLWVLLLWVPGSTGDIQMTQSPSSLSASVGDRVTI | Exemplary chimeric D2E7 |
| | TCRASQGIRNYLAWYQQKPGKAPKLLIYAASTLQSGVPSRF | anti-TNFα variable light |
| | SGSGSGTDFTLTISSLQPEDVATYYCQRYNRAPYTFGQGTK | chain and feline kappa |
| | VEIKRSDAQPSVFLFQPSLDELHTGSASIVCILNDFYPKEV | constant light chain with |
| | NVKWKVDGVVQNKGIQESTTEQNSKDSTYSLSSTLTMSSTE | leader sequence |
| | YQSHEKFSCEVTHKSLASTLVKSFNRSECQRE | |
| 117 | DIQMTQSPSSLSASVGDRVTITCRASQGIRNYLAWYQQKPG | Exemplary chimeric D2E7 |
| | KAPKLLIYAASTLQSGVPSRFSGSGSGTDFTLTISSLQPED | anti-TNFα variable light |
| | VATYYCQRYNRAPYTFGQGTKVEIKRSDAQPSVFLFQPSLD | chain and feline kappa |
| | ELHTGSASIVCILNDFYPKEVNVKWKVDGVVQNKGIQESTT | constant light chain |
| | EQNSKDSTYSLSSTLTMSSTEYQSHEKFSCEVTHKSLASTL | without leader sequence |
| | VKSFNRSECQRE | |
| 118 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and wild-type feline |
| | ASSLDYWGQGTLVTVSSASTTAPSVFPLAPSCGTTSGATVA | IgG1b Fc with leader |
| | LACLVLGYFPEPVTVSWNSGALTSGVHTFPAVLQASGLYSL | sequence |
| | SSMVTVPSSRWLSDTFTCNVAHPPSNTKVDKTVRKTDHPPG | |
| | PKPCDCPKCPPPEMLGGPSIFIFPPKPKDTLSISRTPEVTC | |
| | LVVDLGPDDSDVQITWFVDNTQVYTAKTSPREEQFNSTYRV | |
| | VSVLPILHQDWLKGKEFKCKVNSKSLPSPIERTISKAKGQP | |
| | HEPQVYVLPPAQEELSRNKVSVTCLIKSFHPPDIAVEWEIT | |
| | GQPEPENNYRTTPPQLDSDGTYFVYSKLSVDRSHWQRGNTY | |
| | TCSVSHEALHSHHTQKSLTQSPGK | |
| 119 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and wild-type feline |
| | TTAPSVFPLAPSCGTTSGATVALACLVLGYFPEPVTVSWNS | IgG1b Fc without leader |
| | GALTSGVHTFPAVLQASGLYSLSSMVTVPSSRWLSDTFTCN | sequence |
| | VAHPPSNTKVDKTVRKTDHPPGPKPCDCPKCPPPEMLGGPS | |
| | IFIFPPKPKDTLSISRTPEVTCLVVDLGPDDSDVQITWFVD | |
| | NTQVYTAKTSPREEQFNSTYRVVSVLPILHQDWLKGKEFKC | |
| | KVNSKSLPSPIERTISKAKGQPHEPQVYVLPPAQEELSRNK | |
| | VSVTCLIKSFHPPDIAVEWEITGQPEPENNYRTTPPQLDSD | |
| | GTYFVYSKLSVDRSHWQRGNTYTCSVSHEALHSHHTQKSLT | |
| | QSPGK | |
| 120 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant feline |
| | ASSLDYWGQGTLVTVSSASTTAPSVFPLAPSCGTTSGATVA | IgG1b Fc (YTE) with |
| | LACLVLGYFPEPVTVSWNSGALTSGVHTFPAVLQASGLYSL | leader sequence |
| | SSMVTVPSSRWLSDTFTCNVAHPPSNTKVDKTVRKTDHPPG | |
| | PKPCDCPKCPPPEMLGGPSIFIFPPKPKDTLYITREPEVTC | |
| | LVVDLGPDDSDVQITWFVDNTQVYTAKTSPREEQFNSTYRV | |
| | VSVLPILHQDWLKGKEFKCKVNSKSLPSPIERTISKAKGQP | |
| | HEPQVYVLPPAQEELSRNKVSVTCLIKSFHPPDIAVEWEIT | |
| | GQPEPENNYRTTPPQLDSDGTYFVYSKLSVDRSHWQRGNTY | |
| | TCSVSHEALHSHHTQKSLTQSPGK | |
| 121 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant feline |
| | TTAPSVFPLAPSCGTTSGATVALACLVLGYFPEPVTVSWNS | IgG1 Fc (YTE) without |
| | GALTSGVHTFPAVLQASGLYSLSSMVTVPSSRWLSDTFTCN | leader sequence |
| | VAHPPSNTKVDKTVRKTDHPPGPKPCDCPKCPPPEMLGGPS | |
| | IFIFPPKPKDTLYITREPEVTCLVVDLGPDDSDVQITWFVD | |
| | NTQVYTAKTSPREEQFNSTYRVVSVLPILHQDWLKGKEFKC | |
| | KVNSKSLPSPIERTISKAKGQPHEPQVYVLPPAQEELSRNK | |
| | VSVTCLIKSFHPPDIAVEWEITGQPEPENNYRTTPPQLDSD | |
| | GTYFVYSKLSVDRSHWQRGNTYTCSVSHEALHSHHTQKSLT | |
| | QSPGK | |
| 122 | METDTLLLWVLLLWVPGSTGDIQMTQSPSSLSASVGDRVTI | Exemplary chimeric D2E7 |
| | TCRASQGIRNYLAWYQQKPGKAPKLLIYAASTLQSGVPSRF | anti-TNFα variable light |
| | SGSGSGTDFTLTISSLQPEDVATYYCQRYNRAPYTFGQGTK | chain and equine kappa |
| | VEIKRDDAKPSAFIFPPSSEELSSGSASVVCLVYGFYPSGA | constant light chain with |
| | TINWKVDGLAKTSSFHSSLTEQDSKDNTYSLSSTLTLPKAD | leader sequence |
| | YEAHNVYACEVSHKTLSSPLVKSFNREDC | |
| 123 | DIQMTQSPSSLSASVGDRVTITCRASQGIRNYLAWYQQKPG | Exemplary chimeric D2E7 |
| | KAPKLLIYAASTLQSGVPSRFSGSGSGTDFTLTISSLQPED | anti-TNFα variable light |
| | VATYYCQRYNRAPYTFGQGTKVEIKRDDAKPSAFIFPPSSE | chain and equine kappa |
| | ELSSGSASVVCLVYGFYPSGATINWKVDGLAKTSSFHSSLT | constant light chain |
| | EQDSKDNTYSLSSTLTLPKADYEAHNVYACEVSHKTLSSPL | without leader sequence |
| | VKSFNREDC | |
| 124 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and wild-type equine |
| | ASSLDYWGQGTLVTVSSASTTAPKVFALAPGCGTTSDSTVA | IgG1 Fc with leader |
| | LGCLVSGYFPEPVKVSWNSGSLTSGVHTFPSVLQSSGFYSL | sequence |
| | SSMVTVPASSWTSETYICNVVHAASNFKVDKRIEPIPDNHQ | |
| | KVCDMSKCPKCPAPELLGGPSVFIFPPNPKDTLMITRTPEV | |
| | TCVVVDVSQENPDVKFNWYMDGVEVRTATTRPKEEQFNSTY | |
| | RVVSVLRIQHQDWLSGKEFKCKVNNQALPQPIERTITKTKG | |
| | RSQEPQVYVLAPHPDELSKSKVSVTCLVKDFYPPEINIEWQ | |
| | SNGQPELETKYSTTQAQQDSDGSYFLYSKLSVDRNRWQQGT | |
| | TFTCGVMHEALHNHYTQKNVSKNPGK | |
| 125 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and wild-type equine |
| | TTAPKVFALAPGCGTTSDSTVALGCLVSGYFPEPVKVSWNS | IgG1 Fc without leader |
| | GSLTSGVHTFPSVLQSSGFYSLSSMVTVPASSWTSETYICN | sequence |
| | VVHAASNFKVDKRIEPIPDNHQKVCDMSKCPKCPAPELLGG | |
| | PSVFIFPPNPKDTLMITRTPEVTCVVVDVSQENPDVKFNWY | |
| | MDGVEVRTATTRPKEEQFNSTYRVVSVLRIQHQDWLSGKEF | |
| | KCKVNNQALPQPIERTITKTKGRSQEPQVYVLAPHPDELSK | |
| | SKVSVTCLVKDFYPPEINIEWQSNGQPELETKYSTTQAQQD | |
| | SDGSYFLYSKLSVDRNRWQQGTTFTCGVMHEALHNHYTQKM | |
| | VSKNPGK | |
| 126 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant equine |
| | ASSLDYWGQGTLVTVSSASTTAPKVFALAPGCGTTSDSTVA | IgG1 Fc (F00) with leader |
| | LGCLVSGYFPEPVKVSWNSGSLTSGVHTFPSVLQSSGFYSL | sequence |
| | SSMVTVPASSWTSETYICNVVHAASNFKVDKRIEPIPDNHQ | |
| | KVCDMSKCPKCPAPELLGGPSVFIFPPNPKDTLFITRTPEV | |
| | TCVVVDVSQENPDVKFNWYMDGVEVRTATTRPKEEQFNSTY | |
| | RVVSVLRIQHQDWLSGKEFKCKVNNQALPQPIERTITKTKG | |
| | RSQEPQVYVLAPHPDELSKSKVSVTCLVKDFYPPEINIEWQ | |
| | SNGQPELETKYSTTQAQQDSDGSYFLYSKLSVDRNRWQQGT | |
| | TFTCGVMHEALHNHYTQKNVSKNPGK | |
| 127 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant equine |
| | TTAPKVFALAPGCGTTSDSTVALGCLVSGYFPEPVKVSWNS | IgG1 Fc (F00) without |
| | GSLTSGVHTFPSVLQSSGFYSLSSMVTVPASSWTSETYICN | leader sequence |
| | VVHAASNFKVDKRIEPIPDNHQKVCDMSKCPKCPAPELLGG | |
| | PSVFIFPPNPKDTLFITRTPEVTCVVVDVSQENPDVKFNWY | |
| | MDGVEVRTATTRPKEEQFNSTYRVVSVLRIQHQDWLSGKEF | |
| | KCKVNNQALPQPIERTITKTKGRSQEPQVYVLAPHPDELSK | |
| | SKVSVTCLVKDFYPPEINIEWQSNGQPELETKYSTTQAQQD | |
| | SDGSYFLYSKLSVDRNRWQQGTTFTCGVMHEALHNHYTQKN | |
| | VSKNPGK | |
| 128 | MAVLGLLLCLVTFPSCVLSEVQLVESGGGLVQPGRSLRLSC | Exemplary chimeric D2E7 |
| | AASGFTFDDYAMHWVRQAPGKGLEWVSAITWNSGHIDYADS | anti-TNFα variable heavy |
| | VEGRFTISRDNAKNSLYLQMNSLRAEDTAVYYCAKVSYLST | chain and variant equine |
| | ASSLDYWGQGTLVTVSSASTTAPKVFALAPGCGTTSDSTVA | IgG1 Fc (Y0E) with leader |
| | LGCLVSGYFPEPVKVSWNSGSLTSGVHTFPSVLQSSGFYSL | sequence |
| | SSMVTVPASSWTSETYICNVVHAASNFKVDKRIEPIPDNHQ | |
| | KVCDMSKCPKCPAPELLGGPSVFIFPPNPKDTLYITREPEV | |
| | TCVVVDVSQENPDVKFNWYMDGVEVRTATTRPKEEQFNSTY | |
| | RVVSVLRIQHQDWLSGKEFKCKVNNQALPQPIERTITKTKG | |
| | RSQEPQVYVLAPHPDELSKSKVSVTCLVKDFYPPEINIEWQ | |
| | SNGQPELETKYSTTQAQQDSDGSYFLYSKLSVDRNRWQQGT | |
| | TFTCGVMHEALHNHYTQKNVSKNPGK | |
| 129 | EVQLVESGGGLVQPGRSLRLSCAASGFTFDDYAMHWVRQAP | Exemplary chimeric D2E7 |
| | GKGLEWVSAITWNSGHIDYADSVEGRFTISRDNAKNSLYLQ | anti-TNFα variable heavy |
| | MNSLRAEDTAVYYCAKVSYLSTASSLDYWGQGTLVTVSSAS | chain and variant equine |
| | TTAPKVFALAPGCGTTSDSTVALGCLVSGYFPEPVKVSWNS | IgG1 Fc (Y0E) without |
| | GSLTSGVHTFPSVLQSSGFYSLSSMVTVPASSWTSETYICN | leader sequence |
| | VVHAASNFKVDKRIEPIPDNHQKVCDMSKCPKCPAPELLGG | |
| | PSVFIFPPNPKDTLYITREPEVTCVVVDVSQENPDVKFNWY | |
| | MDGVEVRTATTRPKEEQFNSTYRVVSVLRIQHQDWLSGKEF | |
| | KCKVNNQALPQPIERTITKTKGRSQEPQVYVLAPHPDELSK | |
| | SKVSVTCLVKDFYPPEINIEWQSNGQPELETKYSTTQAQQD | |
| | SDGSYFLYSKLSVDRNRWQQGTTFTCGVMHEALHNHYTQKN | |
| | VSKNPGK | |

### DESCRIPTION OF CERTAIN EMBODIMENTS

Variant IgG Fc polypeptides derived from companion animals, such as canine, equine, and feline, having altered binding to FcRn are described. In some embodiments, antibodies, antibody fragments, or fusion proteins comprise a variant IgG Fc polypeptide. Methods of producing or purifying variant IgG Fc polypeptides and methods of administering variant IgG Fc polypeptides to companion animals are also provided.

For the convenience of the reader, the following definitions of terms used herein are provided.

As used herein, numerical terms such as KD are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### Exemplary Variant IgG Fc Polypeptides

Novel variant IgG Fc polypeptides are provided, for example, variant IgG Fc polypeptides with altered binding affinity to FcRn, such as enhanced binding affinity to FcRn at an acidic pH (e.g., at a pH in the range of from about 5.0 to about 6.5).

“Amino acid sequence,” means a sequence of amino acids residues in a peptide or protein. The terms “polypeptide” and “protein” are used interchangeably to refer to a polymer of amino acid residues, and are not limited to a minimum length. Such polymers of amino acid residues may contain natural or unnatural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“IgX Fc” or “IgX Fc polypeptide” refers to an Fc polypeptide derived from a particular antibody isotype (e.g., IgG, IgA, IgD, IgE, IgM, etc.), where “X” denotes the antibody isotype. Thus, “IgG Fc” denotes that the Fc polypeptide is derived from a γ chain, “IgA Fc” denotes that the Fc polypeptide is derived from an α chain, “IgD Fc” denotes that the Fc polypeptide is derived from a δ chain, “IgE Fc” denotes that the Fc polypeptide is derived from a ε chain, “IgM Fc” denotes that the Fc polypeptide is derived from a μ chain, etc. In some embodiments, the IgG Fc polypeptide comprises the hinge, CH2, and CH3, but does not comprise CH1 or CL. In some embodiments, the IgG Fc polypeptide comprises CH2 and CH3, but does not comprise CH1, the hinge, or CL. In some embodiments, the IgG Fc polypeptide comprises CH1, hinge, CH2, and CH3, with or without CL1. In some embodiments, an Fc polypeptide, such as an IgG Fc polypeptide, lacks one or more C-terminal amino acids, such as 1 to 20, 1 to 15, 1 to 10, 1 to 5, or 1 to 2 amino acids, while retaining biological activity. In some embodiments, the biological activity of is the ability to bind FcRn. An “effector function” of the Fc polypeptide is an action or activity performed in whole or in part by any antibody in response to a stimulus and may include complement fixation and/or ADCC (antibody-dependent cellular cytotoxicity) induction. “IgX-N Fc” denotes that the Fc polypeptide is derived from a particular subclass of antibody isotype (such as canine IgG subclass IgG-A, IgG-B, IgG-C, or IgG-D; feline IgG subclass IgG-1a, IgG-1b, or IgG-2; or equine IgG subclass IgG-1, IgG-2, IgG-3, IgG-4, IgG-5, IgG-6, or IgG-7, etc.), where “N” denotes the subclass.

In some embodiments, a companion animal species is a canine (or dog), a feline (or cat), or an equine (or horse). In some embodiments, a companion animal species is a small mammal, such as a canine, feline, dog, cat, rabbit, ferret, guinea pig, rodent, etc. In some embodiments, a companion animal species is a farm animal, such as a horse, cow, pig, etc.

In some embodiments, an IgX Fc polypeptide or an IgX-N Fc polypeptide is derived from a companion animal, such as a dog, a cat, or a horse. In some embodiments, IgG Fc polypeptides are isolated from canine γ heavy chains, such as IgG-A, IgG-B, IgG-C, or IgG-D. In some instances, IgG Fc polypeptides are isolated from feline γ heavy chains, such as IgG1 (e.g., IgG1a or IgG1b) or IgG2. In other instances, IgG Fc polypeptides are isolated from equine γ heavy chains, such as IgG-1, IgG-2, IgG-3, IgG-4, IgG-5, IgG-6, or IgG-7.

The terms “IgX Fc” and “IgX Fc polypeptide” include wild-type IgX Fc polypeptides and variant IgX Fc polypeptides, unless indicated otherwise.

“Wild-type” refers to a non-mutated version of a polypeptide that occurs in nature, or a fragment thereof. A wild-type polypeptide may be produced recombinantly.

In some embodiments, a wild-type IgG Fc polypeptide comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

A “variant” is a polypeptide that differs from a reference polypeptide by single or multiple non-native amino acid substitutions, deletions, and/or additions. In some embodiments, a variant retains at least one biological activity of the reference polypeptide. In some embodiments, a variant has a biological activity that the reference polypeptide substantially lacks.

A “variant IgG Fc” as used herein is an IgG Fc polypeptide that differs from a reference IgG Fc polypeptide by single or multiple amino acid substitutions, deletions, and/or additions and substantially retains at least one biological activity of the reference IgG Fc polypeptide.

In some embodiments, a variant IgG Fc polypeptide comprises a variant IgG Fc polypeptide of a companion animal species. In some embodiments, a variant IgG Fc polypeptide comprises a variant canine IgG Fc polypeptide, a variant equine IgG Fc polypeptide, or a feline IgG Fc polypeptide. In some embodiments, the variant IgG Fc polypeptide is a variant canine IgG-A Fc polypeptide, a variant canine IgG-B Fc polypeptide, a variant canine IgG-C Fc polypeptide, or a variant canine IgG-D Fc polypeptide. In some embodiments, the variant IgG Fc polypeptide is a variant equine IgG1 Fc polypeptide, a variant equine IgG2 Fc polypeptide, a variant equine IgG3 Fc polypeptide, a variant equine IgG4 Fc polypeptide, a variant equine IgG5 Fc polypeptide, a variant equine IgG6 Fc polypeptide, or a variant equine IgG7 Fc polypeptide. In some embodiments, the variant IgG Fc polypeptide is a variant feline IgG1 Fc polypeptide or a variant feline IgG2 Fc polypeptide.

As used herein, “percent (%) amino acid sequence identity” and “homology” with respect to a nucleic acid molecule or polypeptide sequence are defined as the percentage of nucleotide or amino acid residues in a reference sequence that are identical with the nucleotide or amino acid residues in the specific nucleic acid molecule or polypeptide sequence, after aligning the sequences and introducing gaps, if necessary to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for measuring alignment, including any parameters needed to achieve maximal alignment over the full length of sequences being compared.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

As used herein, “position corresponding to position n,” wherein n is any number, refers to an amino acid position of a subject polypeptide that aligns with position n of a reference polypeptide after aligning the amino acid sequences of the subject and reference polypeptides and introducing gaps. Alignment for purposes of whether a position of a subject polypeptide corresponds with position n of a reference polypeptide can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for alignment, including any parameters needed to achieve maximal alignment over the full length of two sequences being compared. In some embodiments, the subject polypeptide and the reference polypeptide are of different lengths.

An “point mutation” is a mutation that involves a single amino acid residue. The mutation may be the loss of an amino acid, substitution of one amino acid residue for another, or the insertion of an additional amino acid residue.

An “amino acid substitution” refers to the replacement of one amino acid in a polypeptide with another amino acid. In some embodiments, an amino acid substitution is a conservative substitution. Nonlimiting exemplary conservative amino acid substitutions are shown in Table 2. Amino acid substitutions may be introduced into a molecule of interest and the products screened for a desired activity, for example, retained/improved antigen binding, decreased immunogenicity, or improved ADCC or CDC or enhanced pharmacokinetics.

TABLE 2

| Original Residue | Exemplary Substitutions |
|---|---|
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

An “amino acid derivative,” as used herein, refers to any amino acid, modified amino acid, and/or amino acid analogue, that is not one of the 20 common natural amino acids found in humans. Exemplary amino acid derivatives include natural amino acids not found in humans (e.g., seleno cysteine and pyrrolysine, which may be found in some microorganisms) and unnatural amino acids. Exemplary amino acid derivatives, include, but are not limited to, amino acid derivatives commercially available through chemical product manufacturers (e.g., sigmaaldrich.com/chemistry/chemistry-products.html?TablePage=16274965, accessed on May 6, 2017, which is incorporated herein by reference). One or more amino acid derivatives may be incorporated into a polypeptide at a specific location using a translation system that utilizes host cells, orthogonal aminoacyl-tRNA synthetases derived from eubacterial synthetases, orthogonal tRNAs, and an amino acid derivative. For further descriptions, see, e.g., U.S. Pat. No. 9,624,485.

In some embodiments, a variant IgG Fc polypeptide comprises an amino acid substitution with an amino acid derivative. In some embodiments, the amino acid derivative is an alanine derivative, a cysteine derivative, an aspartic acid derivative, a glutamic acid derivative, a phenylalanine derivative, a glycine derivative, a histidine derivative, an isoleucine derivative, a lysine derivative, a leucine derivative, a methionine derivative, an asparagine derivative, a proline derivative, a glutamine derivative, an arginine derivative, a serine derivative, a threonine derivative, a valine derivative, a tryptophan derivative, or a tyrosine derivative.

### Exemplary Variant IgG Fc Polypeptides With Modified FcRn Binding

In some embodiments, a variant IgG Fc polypeptide (e.g., a variant canine IgG Fc polypeptide, a variant equine IgG Fc polypeptide, or a variant feline IgG Fc polypeptide) has modified FcRn binding affinity compared to a reference polypeptide. In some embodiments, a variant IgG Fc polypeptide has increased FcRn binding affinity at an acidic pH (e.g., at a pH in the range of from about 5.0 to about 6.5, such as at a pH of about 5.0, a pH of about 5.5, a pH of about 6.0, or a pH of about 6.5) compared to a reference polypeptide.

In some embodiments, a variant IgG Fc polypeptide is at least 80% identical, at least 85% identical, at least 90% identical, at least 95% identical, at least 97% identical, at least 98% identical, or at least 99% identical to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, or SEQ ID NO: 91.

In some embodiments, a variant IgG Fc polypeptide comprises at least one amino acid substitution at at least one position corresponding to position 9, 18, 22, 23, 24, 25, 26, 27, 28, 30, 31, 60, 79, 80, 83, 132, 149, 162, 183, 203, 205, and/or 209 of SEQ ID NO: 1. In some embodiments, a variant IgG Fc polypeptide comprises at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 2. In some embodiments, a variant IgG Fc polypeptide comprises at least one amino acid substitution at at least one position corresponding to position 9, 18, 22, 23, 24, 26, 27, 28, 30, 31, 60, 79, 80, 81, 83, 132, 148, 162, 182, 202, 204, and/or 208 of SEQ ID NO: 3. In some embodiments, a variant IgG Fc polypeptide comprises at least one amino acid substitution at at least one position corresponding to position 9, 18, 22, 23, 24, 25, 26, 27, 28, 30, 60, 79, 80, 83, 132, 149, 163, 183, 203, 205, and/or 209 of SEQ ID NO: 4. In some embodiments, a variant IgG Fc polypeptide comprises at least one amino acid substitution at at least one position corresponding to position 9, 18, 22, 23, 24, 25, 26, 27, 28, 30, 31, 79, 80, 81, 83, 132, 148, 162, 182, 202, 204, 208, and/or 213 of SEQ ID NO: 5. In some embodiments, a variant IgG Fc polypeptide comprises at least one amino acid substitution at at least one position corresponding to position 9, 18, 22, 23, 24, 25, 26, 27, 28, 79, 80, 81, 83, 132, 148, 162, 182, 202, 204, 208, and/or 213 of SEQ ID NO: 6. In some embodiments, a variant IgG Fc polypeptide comprises at least one amino acid substitution at at least one position corresponding to position 9, 18, 22, 23, 24, 25, 27, 28, 30, 31, 60, 79, 80, 81, 83, 132, 148, 162, 182, 202, 204, 208, and/or 213 of SEQ ID NO: 7. In some embodiments, a variant IgG Fc polypeptide comprises at least one amino acid substitution at at least one position corresponding to position 9, 18, 22, 23, 24, 25, 27, 28, 30, 31, 79, 80, 83, 132, 148, 162, 182, 202, 204, 208, and/or 213 of SEQ ID NO: 8. In some embodiments, a variant IgG Fc polypeptide comprises at least one amino acid substitution at at least one position corresponding to position 9, 18, 22, 23, 24, 25, 27, 28, 30, 31, 79, 80, 81, 83, 132, 148, 162, 182, 202, 204, 208 and/or 213 of SEQ ID NO: 9. In some embodiments, a variant IgG Fc polypeptide comprises at least one amino acid substitution at at least one position corresponding to position 18, 22, 23, 24, 25, 27, 28, 30, 31, 79, 80, 81, 83, 132, 148, 162, 182, 202, 204, 208, and/or 213 of SEQ ID NO: 10. In some embodiments, a variant IgG Fc polypeptide comprises at least one amino acid substitution at at least one position corresponding to position 18, 22, 23, 24, 25, 27, 28, 30, 31, 79, 80, 81, 83, 132, 148, 162, 182, 202, 204, 208, and/or 213 of SEQ ID NO: 11. In some embodiments, a variant IgG Fc polypeptide comprises at least one amino acid substitution at at least one position corresponding to position 9, 18, 22, 23, 24, 25, 26, 27, 28, 30, 31, 60, 79, 80, 81, 83, 148, 162, 182, 202, 204, 208, and/or 213 of SEQ ID NO: 12. In some embodiments, a variant IgG Fc polypeptide comprises at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 13. In some embodiments, a variant IgG Fc polypeptide comprises at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 54.

In some embodiments, a variant IgG Fc polypeptide comprises at least one amino acid substitution at at least one of position selected from position 9, 18, 22, 23, 24, 25, 26, 27, 28, 30, 31, 60, 79, 80, 83, 132, 149, 162, 183, 203, 205, and/or 209 of SEQ ID NO: 1. In some embodiments, a variant IgG Fc polypeptide comprises at least one amino acid substitution at at least one of position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 2. In some embodiments, a variant IgG Fc polypeptide comprises at least one amino acid substitution at at least one of position selected from position 9, 18, 22, 23, 24, 26, 27, 28, 30, 31, 60, 79, 80, 81, 83, 132, 148, 162, 182, 202, 204, and/or 208 of SEQ ID NO: 3. In some embodiments, a variant IgG Fc polypeptide comprises at least one amino acid substitution at at least one of position selected from position 9, 18, 22, 23, 24, 25, 26, 27, 28, 30, 60, 79, 80, 83, 132, 149, 163, 183, 203, 205, and/or 209 of SEQ ID NO: 4. In some embodiments, a variant IgG Fc polypeptide comprises at least one amino acid substitution at at least one of position selected from position 9, 18, 22, 23, 24, 25, 26, 27, 28, 30, 31, 80, 81, 83, 132, 148, 162, 182, 202, 204, 208, and/or 213 of SEQ ID NO: 5. In some embodiments, a variant IgG Fc polypeptide comprises at least one amino acid substitution at at least one of position selected from position 9, 18, 22, 23, 24, 25, 26, 27, 28, 79, 80, 81, 83, 132, 148, 162, 182, 202, 204, 208, and/or 213 of SEQ ID NO: 6. In some embodiments, a variant IgG Fc polypeptide comprises at least one amino acid substitution at at least one of position selected from position 9, 18, 22, 23, 24, 25, 27, 28, 30, 31, 60, 80, 81, 83, 132, 148, 162, 182, 202, 204, 208, and/or 213 of SEQ ID NO: 7. In some embodiments, a variant IgG Fc polypeptide comprises at least one amino acid substitution at at least one of position selected from position 9, 18, 22, 23, 24, 25, 27, 28, 30, 31, 79, 80, 83, 132, 148, 162, 182, 202, 204, 208, and/or 213 of SEQ ID NO: 8. In some embodiments, a variant IgG Fc polypeptide comprises at least one amino acid substitution at at least one of position selected from position 9, 18, 22, 23, 24, 25, 27, 28, 30, 31, 79, 80, 81, 83, 132, 148, 162, 182, 202, 204, 208 and/or 213 of SEQ ID NO: 9. In some embodiments, a variant IgG Fc polypeptide comprises at least one amino acid substitution at at least one of position selected from position 18, 22, 23, 24, 25, 27, 28, 30, 31, 79, 80, 81, 83, 132, 148, 162, 182, 202, 204, 208, and/or 213 of SEQ ID NO: 10. In some embodiments, a variant IgG Fc polypeptide comprises at least one amino acid substitution at at least one of position selected from position 18, 22, 23, 24, 25, 27, 28, 30, 31, 80, 81, 83, 132, 148, 162, 182, 202, 204, 208, and/or 213 of SEQ ID NO: 11. In some embodiments, a variant IgG Fc polypeptide comprises at least one amino acid substitution at at least one of position selected from position 9, 18, 22, 23, 24, 25, 26, 27, 28, 30, 31, 60, 79, 80, 81, 83, 148, 162, 182, 202, 204, 208, and/or 213 of SEQ ID NO: 12. In some embodiments, a variant IgG Fc polypeptide comprises at least one amino acid substitution at at least one of position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 13. In some embodiments, a variant IgG Fc polypeptide comprises at least one amino acid substitution at at least one position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 54.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 149; a leucine, a phenylalanine, or a tyrosine at a position corresponding to position 162; an isoleucine at a position corresponding to position 183; a leucine or a tryptophan at a position corresponding to position 203; a proline at a position corresponding to position 205; and/or a serine, an alanine, an arginine, a phenylalanine, a tyrosine, a histidine, or a tryptophan at a position corresponding to position 209 of SEQ ID NO: 1.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, tyrosine, methionine, arginine, or valine at a position corresponding to position 24; a threonine at a position corresponding to position 25; a tryptophan, a tyrosine, a histidine, a leucine, a phenylalanine, a valine, a methionine, a threonine, or a serine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 2.

In some embodiments, a variant IgG Fc polypeptide comprises a tyrosine at a position corresponding to position 9; a proline at a position corresponding to position 18; a histidine or a proline at a position corresponding to position 22; a valine, an arginine, or a lysine at a position corresponding to position 23; a phenylalanine, a tyrosine, a methionine, or an arginine at a position corresponding to position 24; a tryptophan, a tyrosine, a histidine, a leucine, a phenylalanine, a valine, a methionine, a threonine, or a serine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; and/or a serine, an alanine, an arginine, a phenylalanine, a tyrosine, a histidine, or a tryptophan at a position corresponding to position 208 of SEQ ID NO: 3.

In some embodiments, a variant IgG Fc polypeptide comprises a tyrosine at a position corresponding to position 9; a proline at a position corresponding to position 18; a histidine or a proline at a position corresponding to position 22; a valine, an arginine, or a lysine at a position corresponding to position 23; a phenylalanine, a tyrosine, a methionine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an alanine at a position corresponding to position 60; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 149; a leucine, a phenylalanine, or a tyrosine at a position corresponding to position 163; an isoleucine at a position corresponding to position 183; a leucine or a tryptophan at a position corresponding to position 203; a proline at a position corresponding to position 205; and/or a serine, an alanine, an arginine, a phenylalanine, a tyrosine, a histidine, or a tryptophan at a position corresponding to position 209 of SEQ ID NO: 4.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, a glutamic acid, or a methionine at a position corresponding to position 79, a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 5.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, a methionine, or a threonine at a position corresponding to position 26; a leucine, a methionine, a proline, an isoleucine, an asparagine, or a valine at a position corresponding to position 27; a methionine, a glutamic acid, an asparagine, a histidine, an isoleucine, or a valine at a position corresponding to position 28; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83, an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 6.

In some embodiments, a variant IgG Fc polypeptide comprises a tyrosine at a position corresponding to position 9; a proline at a position corresponding to position 18; a histidine, an isoleucine, and/or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, a glutamic acid, or a methionine at a position corresponding to position 79, a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 7.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 8.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 9.

In some embodiments, a variant IgG Fc polypeptide comprises a proline at a position corresponding to position 18; a histidine, an isoleucine, or a proline at a position corresponding to position 22; a valine, an arginine, or a lysine at a position corresponding to position 23; a phenylalanine, a tyrosine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, or a tryptophan at a position corresponding to position 208; and/or an isoleucine or a valine at a position corresponding to position 213 of SEQ ID NO: 10.

In some embodiments, a variant IgG Fc polypeptide comprises a proline at a position corresponding to position 18; a histidine, an isoleucine, or a proline at a position corresponding to position 22; a valine, an arginine, or a lysine at a position corresponding to position 23; a phenylalanine, a tyrosine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, a glutamic acid, or a methionine at a position corresponding to position 79, a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, a phenylalanine, a tyrosine, a histidine, or a tryptophan at a position corresponding to position 208; and/or an isoleucine or a valine at a position corresponding to position 213 of SEQ ID NO: 11.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 12.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 13.

In some embodiments, a variant IgG Fc polypeptide comprises a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 54.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glycine, an arginine, or an alanine at position 79; a histidine or a phenylalanine at position 80; an alanine or a tyrosine at position 83; an isoleucine at position 132; a valine at position 149; a leucine, a phenylalanine, or a tyrosine at position 162; an isoleucine at position 183; a leucine or a tryptophan at position 203; a proline at position 205; and/or a serine, an alanine, an arginine, a phenylalanine, a tyrosine, a histidine, or a tryptophan at position 209 of SEQ ID NO: 1.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, tyrosine, methionine, arginine, or valine at position 24; a threonine at position 25; a tryptophan, a tyrosine, a histidine, a leucine, a phenylalanine, a valine, a methionine, a threonine, or a serine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 2.

In some embodiments, a variant IgG Fc polypeptide comprises a tyrosine at position 9; a proline at position 18; a histidine or a proline at position 22; a valine, an arginine, or a lysine at position 23; a phenylalanine, a tyrosine, a methionine, or an arginine at position 24; a tryptophan, a tyrosine, a histidine, a leucine, a phenylalanine, a valine, a methionine, a threonine, or a serine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; and/or a serine, an alanine, an arginine, a phenylalanine, a tyrosine, a histidine, or a tryptophan at position 208 of SEQ ID NO: 3.

In some embodiments, a variant IgG Fc polypeptide comprises a tyrosine at position 9; a proline at position 18; a histidine or a proline at position 22; a valine, an arginine, or a lysine at position 23; a phenylalanine, a tyrosine, a methionine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an alanine at position 60; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 149; a leucine, a phenylalanine, or a tyrosine at position 163; an isoleucine at position 183; a leucine or a tryptophan at position 203; a proline at position 205; and/or a serine, an alanine, an arginine, a phenylalanine, a tyrosine, a histidine, or a tryptophan at position 209 of SEQ ID NO: 4.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, a glutamic acid, or a methionine at position 79, a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 5.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, a methionine, or a threonine at position 26; a leucine, a methionine, a proline, an isoleucine, an asparagine, or a valine at position 27; a methionine, a glutamic acid, an asparagine, a histidine, an isoleucine, or a valine at position 28; a glutamine, an arginine, an alanine, a glutamic acid, or a methionine at position 79; a histidine or a phenylalanine at position 80; a glutamic acid at position 81; an alanine, a tyrosine, or a phenylalanine at position 83, an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 6.

In some embodiments, a variant IgG Fc polypeptide comprises a tyrosine at position 9; a proline at position 18; a histidine, an isoleucine, and/or a proline at position 22; a valine, an arginine, or a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, a glutamic acid, or a methionine at position 79, a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 7.

In some embodiments, a variant IgG Fc polypeptide comprises a tyrosine at position 9; a proline at position 18; a histidine, an isoleucine, or a proline at position 22; a valine, an arginine, a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 8.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 9.

In some embodiments, a variant IgG Fc polypeptide comprises a proline at position 18; a histidine, an isoleucine, or a proline at position 22; a valine, an arginine, or a lysine at position 23; a phenylalanine, a tyrosine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, or a tryptophan at position 208; and/or an isoleucine or a valine at position 213 of SEQ ID NO: 10.

In some embodiments, a variant IgG Fc polypeptide comprises a proline at position 18; a histidine, an isoleucine, or a proline at position 22; a valine, an arginine, or a lysine at position 23; a phenylalanine, a tyrosine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, a glutamic acid, or a methionine at position 79, a histidine or a phenylalanine at position 80; a glutamic acid at position 81; an alanine, a tyrosine, or phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, a phenylalanine, a tyrosine, a histidine, or a tryptophan at position 208; and/or an isoleucine or a valine at position 213 of SEQ ID NO: 11.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; a valine at position 148; a leucine, a phenylalanine, or a tyrosine at position 162; an isoleucine at position 182; a leucine or a tryptophan at position 202; a proline at position 204; an alanine, a serine, a phenylalanine, a tyrosine, a histidine, or a tryptophan at position 208; and/or an isoleucine or a valine at position 213 of SEQ ID NO: 12.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or tryptophan at position 202; a proline at position 204; an alanine, an arginine, a phenylalanine, a tyrosine, a histidine, or a tryptophan at position 208; and/or an isoleucine or a valine at position 213 of SEQ ID NO: 13.

In some embodiments, a variant IgG Fc polypeptide comprises a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or tryptophan at position 202; a proline at position 204; an alanine, an arginine, a phenylalanine, a tyrosine, a histidine, or a tryptophan at position 208; and/or an isoleucine or a valine at position 213 of SEQ ID NO: 54.

In some embodiments, a variant IgG Fc polypeptide comprises a phenylalanine or a tyrosine at a position corresponding to position 24, a threonine at a position corresponding to position 26, and/or a glutamic acid at a position corresponding to position 28 of SEQ ID NO: 1. In some embodiments, a variant IgG Fc polypeptide comprises a phenylalanine at a position corresponding to position 24, a threonine at a position corresponding to position 26, and/or a glutamic acid at a position corresponding to position 28 of SEQ ID NO: 2, SEQ ID NO: 3, or SEQ ID NO: 4. In some embodiments, a variant IgG Fc polypeptide comprises a phenylalanine or a tyrosine at a position corresponding to position 24, a threonine at a position corresponding to position 26, and/or a glutamic acid at a position corresponding to position 28 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a phenylalanine or a tyrosine at position 24, a threonine at position 26, and/or a glutamic acid at position 28 of SEQ ID NO: 1. In some embodiments, a variant IgG Fc polypeptide comprises a phenylalanine at position 24, a threonine at position 26, and/or a glutamic acid at position 28 of SEQ ID NO: 2, SEQ ID NO: 3, or SEQ ID NO: 4. In some embodiments, a variant IgG Fc polypeptide comprises a phenylalanine or a tyrosine at position 24, a threonine at position 26, and/or a glutamic acid at position 28 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a phenylalanine at a position corresponding to position 24 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54

In some embodiments, a variant IgG Fc polypeptide comprises a phenylalanine at position 24 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24, a tyrosine at a position corresponding to position 83, and/or an arginine, a phenylalanine, a tyrosine, a histidine, or a tryptophan at a position corresponding to position 209 of SEQ ID NO: 1 or SEQ ID NO: 4. In some embodiments, a variant IgG Fc polypeptide comprises a tyrosine at a position corresponding to position 24, a tyrosine at a position corresponding to position 83, and/or an arginine, a phenylalanine, a tyrosine, a histidine, or a tryptophan at a position corresponding to position 208 of SEQ ID NO: 2 or SEQ ID NO: 3. In some embodiments, a variant IgG Fc polypeptide comprises a tyrosine at a position corresponding to position 24, a tyrosine at a position corresponding to position 83, and/or an arginine, a phenylalanine, a tyrosine, a histidine, or a tryptophan at a position corresponding to position 208 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24, a tyrosine at position 83, and/or an arginine, a phenylalanine, a tyrosine, a histidine, or a tryptophan at position 209 of SEQ ID NO: 1 or SEQ ID NO: 4. In some embodiments, a variant IgG Fc polypeptide comprises a tyrosine at position 24, a tyrosine at position 83, and/or an arginine, a phenylalanine, a tyrosine, a histidine, or a tryptophan at position 208 of SEQ ID NO: 2 or SEQ ID NO: 3. In some embodiments, a variant IgG Fc polypeptide comprises a tyrosine at position 24, a tyrosine at position 83, and/or an arginine, a phenylalanine, a tyrosine, a histidine, or a tryptophan at position 208 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24 and an arginine at a position corresponding to position 209 of SEQ ID NO: 1 or SEQ ID NO: 4. In some embodiments, a variant IgG Fc polypeptide comprises a tyrosine at a position corresponding to position 24 and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24 and a phenylalanine at a position corresponding to position 209 of SEQ ID NO: 1 or SEQ ID NO: 4. In some embodiments, a variant IgG Fc polypeptide comprises a tyrosine at a position corresponding to position 24 and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24 and a tyrosine at a position corresponding to position 209 of SEQ ID NO: 1 or SEQ ID NO: 4. In some embodiments, a variant IgG Fc polypeptide comprises a tyrosine at a position corresponding to position 24 and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24 and a histidine at a position corresponding to position 209 of SEQ ID NO: 1 or SEQ ID NO: 4. In some embodiments, a variant IgG Fc polypeptide comprises a tyrosine at a position corresponding to position 24 and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24 and a tryptophan at a position corresponding to position 209 of SEQ ID NO: 1 or SEQ ID NO: 4. In some embodiments, a variant IgG Fc polypeptide comprises a tyrosine at a position corresponding to position 24 and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 83 and an arginine at a position corresponding to position 209 of SEQ ID NO: 1 or SEQ ID NO: 4. In some embodiments, a variant IgG Fc polypeptide comprises a tyrosine at a position corresponding to position 83 and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 83 and a phenylalanine at a position corresponding to position 209 of SEQ ID NO: 1 or SEQ ID NO: 4. In some embodiments, a variant IgG Fc polypeptide comprises a tyrosine at a position corresponding to position 83 and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 83 and a tyrosine at a position corresponding to position 209 of SEQ ID NO: 1 or SEQ ID NO: 4. In some embodiments, a variant IgG Fc polypeptide comprises a tyrosine at a position corresponding to position 83 and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 83 and a histidine at a position corresponding to position 209 of SEQ ID NO: 1 or SEQ ID NO: 4. In some embodiments, a variant IgG Fc polypeptide comprises a tyrosine at a position corresponding to position 83 and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 83 and a tryptophan at a position corresponding to position 209 of SEQ ID NO: 1 or SEQ ID NO: 4. In some embodiments, a variant IgG Fc polypeptide comprises a tyrosine at a position corresponding to position 83 and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24, a tyrosine at a position corresponding to position 83, and an arginine at a position corresponding to position 209 of SEQ ID NO: 1 or SEQ ID NO: 4. In some embodiments, a variant IgG Fc polypeptide comprises a tyrosine at a position corresponding to position 24, a tyrosine at a position corresponding to position 83, and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24, a tyrosine at a position corresponding to position 83, and a phenylalanine at a position corresponding to position 209 of SEQ ID NO: 1 or SEQ ID NO: 4. In some embodiments, a variant IgG Fc polypeptide comprises a tyrosine at a position corresponding to position 24, a tyrosine at a position corresponding to position 83, and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24, a tyrosine at a position corresponding to position 83, and a tyrosine at a position corresponding to position 209 of SEQ ID NO: 1 or SEQ ID NO: 4. In some embodiments, a variant IgG Fc polypeptide comprises a tyrosine at a position corresponding to position 24, a tyrosine at a position corresponding to position 83, and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24, a tyrosine at a position corresponding to position 83, and a histidine at a position corresponding to position 209 of SEQ ID NO: 1 or SEQ ID NO: 4. In some embodiments, a variant IgG Fc polypeptide comprises a tyrosine at a position corresponding to position 24, a tyrosine at a position corresponding to position 83, and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at a position corresponding to position 24, a tyrosine at a position corresponding to position 83, and a tryptophan at a position corresponding to position 209 of SEQ ID NO: 1 or SEQ ID NO: 4. In some embodiments, a variant IgG Fc polypeptide comprises a tyrosine at a position corresponding to position 24, a tyrosine at a position corresponding to position 83, and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24 and an arginine at position 209 of SEQ ID NO: 1 or SEQ ID NO: 4. In some embodiments, a variant IgG Fc polypeptide comprises a tyrosine at position 24 and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24 and a phenylalanine at position 209 of SEQ ID NO: 1 or SEQ ID NO: 4. In some embodiments, a variant IgG Fc polypeptide comprises a tyrosine at position 24 and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24 and a tyrosine at position 209 of SEQ ID NO: 1 or SEQ ID NO: 4. In some embodiments, a variant IgG Fc polypeptide comprises a tyrosine at position 24 and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24 and a histidine at position 209 of SEQ ID NO: 1 or SEQ ID NO: 4. In some embodiments, a variant IgG Fc polypeptide comprises a tyrosine at position 24 and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24 and a tryptophan at position 209 of SEQ ID NO: 1 or SEQ ID NO: 4. In some embodiments, a variant IgG Fc polypeptide comprises a tyrosine at position 24 and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 83 and an arginine at position 209 of SEQ ID NO: 1 or SEQ ID NO: 4. In some embodiments, a variant IgG Fc polypeptide comprises a tyrosine at position 83 and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 83 and a phenylalanine at position 209 of SEQ ID NO: 1 or SEQ ID NO: 4. In some embodiments, a variant IgG Fc polypeptide comprises a tyrosine at position 83 and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 83 and a tyrosine at position 209 of SEQ ID NO: 1 or SEQ ID NO: 4. In some embodiments, a variant IgG Fc polypeptide comprises a tyrosine at position 83 and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 83 and a histidine at position 209 of SEQ ID NO: 1 or SEQ ID NO: 4. In some embodiments, a variant IgG Fc polypeptide comprises a tyrosine at position 83 and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 83 and a tryptophan at position 209 of SEQ ID NO: 1 or SEQ ID NO: 4. In some embodiments, a variant IgG Fc polypeptide comprises a tyrosine at position 83 and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24, a tyrosine at position 83, and an arginine at position 209 of SEQ ID NO: 1 or SEQ ID NO: 4. In some embodiments, a variant IgG Fc polypeptide comprises a tyrosine at position 24, a tyrosine at position 83, and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24, a tyrosine at position 83, and a phenylalanine at position 209 of SEQ ID NO: 1 or SEQ ID NO: 4. In some embodiments, a variant IgG Fc polypeptide comprises a tyrosine at position 24, a tyrosine at position 83, and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24, a tyrosine at position 83, and a tyrosine at position 209 of SEQ ID NO: 1 or SEQ ID NO: 4. In some embodiments, a variant IgG Fc polypeptide comprises a tyrosine at position 24, a tyrosine at position 83, and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24, a tyrosine at position 83, and a histidine at position 209 of SEQ ID NO: 1 or SEQ ID NO: 4. In some embodiments, a variant IgG Fc polypeptide comprises a tyrosine at position 24, a tyrosine at position 83, and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises a tyrosine at position 24, a tyrosine at position 83, and a tryptophan at position 209 of SEQ ID NO: 1 or SEQ ID NO: 4. In some embodiments, a variant IgG Fc polypeptide comprises a tyrosine at position 24, a tyrosine at position 83, and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

In some embodiments, a variant IgG Fc polypeptide comprises the amino acid sequence of SEQ ID NO: 14, SEQ ID NO: 15, SEQ ID NO: 16, SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48; SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, or SEQ ID NO: 91.

In some embodiments, a polypeptide comprises the amino acid sequence of SEQ ID NO: 14, SEQ ID NO: 15, SEQ ID NO: 16, SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, SEQ ID NO: 51; SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, SEQ ID NO: 91, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 108, SEQ ID NO: 109, SEQ ID NO: 110, SEQ ID NO: 113, SEQ ID NO: 114, SEQ ID NO: 115, SEQ ID NO: 120, SEQ ID NO: 121, SEQ ID NO: 126, SEQ ID NO: 127, SEQ ID NO: 128, or SEQ ID NO: 129.

In some embodiments, a variant IgG Fc polypeptide has modified Protein A binding affinity. In some embodiments, a variant IgG Fc polypeptide has increased binding affinity to Protein A. In some embodiments, a variant IgG Fc polypeptide may be purified using Protein A column chromatography. In some embodiments, a variant IgG Fc polypeptide has modified CD16, CD32, and/or CD64 binding affinity. In some embodiments, a variant IgG Fc polypeptide has decreased binding affinity to CD16, CD32, and/or CD64. In some embodiments, a variant IgG Fc may have a reduced ADCC immune response. In some embodiments, a variant IgG Fc polypeptide has modified C1q binding affinity. In some embodiments, a variant IgG Fc polypeptide has reduced binding affinity to C1q. In some embodiments, a variant IgG Fc polypeptide may have reduced complement fixation. In some embodiments, a variant IgG Fc may have a reduced complement-mediated immune response.

### Exemplary Fusion Molecules

Polypeptides and other molecules may comprise a variant IgG Fc polypeptide. In some embodiments, a fusion molecule comprises a variant IgG Fc polypeptide, such as the variant IgG Fc polypeptides described herein. In some embodiments, an antibody or an antibody fragment comprises a variant IgG Fc polypeptide, such as the variant IgG Fc polypeptides described herein.

A “fusion molecule,” as used herein, refers to a molecule comprising one or more “fusion partners.” In some embodiments, the fusion partners are covalently linked (“fused”). If two fusion partners are both polypeptides, the fusion partner polypeptides may be part of a contiguous amino acid sequence (i.e., a contiguous polypeptide). A first fusion partner polypeptide may be linked to either the N-terminus or the C-terminus of a second fusion partner. In some embodiments, the fusion partners are translated as a single polypeptide from a coding sequence that encodes both fusion partners. Fusion partners may be covalently linked through other means, such as, for example, a chemical linkage other than a peptide bond. Many known methods of covalently linking polypeptides to other molecules (for example, fusion partners) may be used. In other embodiments, the fusion partners are fused through a “linker,” which is comprised of at least one amino acid or chemical moiety. In some embodiments, fusion partners are noncovalently linked. In some such embodiments, they may be linked, for example, using binding pairs. Exemplary binding pairs include, but are not limited to, biotin and avidin or streptavidin, an antibody and its antigen, etc.

In some embodiments, a fusion partner is a therapeutic polypeptide. Exemplary therapeutic polypeptides include, but are not limited to, a late embryogenesis abundant (LEA) polypeptide (e.g., an LEA-3 polypeptide), an NGF (or Nerve Growth Factor) polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα (or Tumor Necrosis Factor Alpha) polypeptide, a receptor of a TNFα polypeptide, a TNFR (or Tumor Necrosis Factor Receptor) polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 (or Interleukin 5) polypeptide, a receptor of an IL5 polypeptide, an IL5R (or Interleukin 5 Receptor) polypeptide (e.g., an ECD of an IL5R polypeptide), an IL5Rα polypeptide (e.g., an ECD of an IL5Rα polypeptide), an IL6 (or Interleukin 6) polypeptide, a receptor of an IL6 polypeptide, an IL6R (or Interleukin 6 Receptor) polypeptide (e.g., an ECD of an IL6R polypeptide), an IL17 (or Interleukin 17) polypeptide, a receptor of an IL17 polypeptide, an IL17R (or Interleukin 17 Receptor) polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 (or Interleukin 23) polypeptide, a receptor of an IL23 polypeptide, an IL23R (or Interleukin 23 Receptor) polypeptide (e.g., an ECD of an IL23R polypeptide), an IL12Rβ1 polypeptide (e.g., an ECD of an IL12Rβ1 polypeptide), a PDL (or Programmed Cell Death Ligand) polypeptide, a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 (or Cytotoxic T-Lymphocyte Associated Protein 4) polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 (or Lymphocyte Activating Gene 3) polypeptide (e.g., an ECD of a LAG-3 polypeptide), an IL31 (or Interleukin 31) polypeptide, a receptor of an IL31 polypeptide, an IL31RA (an Interleukin 31 Receptor A) polypeptide (e.g., an ECD of an IL31RA polypeptide), an OSMR (or Oncostatin M Receptor) polypeptide (e.g., an ECD of an OSMR polypeptide), an IL4 (or Interleukin 4) polypeptide, a receptor of an IL4R polypeptide, an IL4R (or Interleukin 4 Receptor) polypeptide (e.g., an ECD of an IL4R polypeptide), an IL13 (or Interleukin 13 Receptor) polypeptide, a receptor of an IL13 polypeptide, an IL13RA1 (or Interleukin 13 Receptor Al) polypeptide (e.g., an ECD of an IL13RA1 polypeptide), an IL4R (or Interleukin 4 Receptor) polypeptide (e.g., an ECD of an IL4R polypeptide), an IL13Rα2 (or Interleukin 13 Receptor α2) polypeptide (e.g., an ECD of an IL13Rα2 polypeptide), an IL22 (or Interleukin 22) polypeptide, a receptor of an IL22 polypeptide (e.g., an ECD of an IL22 polypeptide), an IL22Rα1 (or Interleukin 22 Receptor α1) polypeptide (e.g., an ECD of an IL22Rα1 polypeptide), an IL10Rβ2 (or Interleukin 10 Receptor β2) polypeptide (e.g., an ECD of an IL10Rβ2 polypeptide), an IL33 (or Interleukin 33) polypeptide, a receptor of an IL33 polypeptide, an IL1RL1 polypeptide (e.g., an ED of an IL1RL1 polypeptide), an EGF (or Epidermal Growth Factor) polypeptide, a receptor of an EGF polypeptide, a TGFα (or Transforming Growth Factor a) polypeptide, a receptor of a TGFα polypeptide, an EGFR (or Epidermal Growth Factor Receptor) polypeptide (e.g., an ECD of an EGFR polypeptide), an MMP9 (or Matrix Metallopeptidase 9) polypeptide, an FGF (or Fibroblast Growth Factor) polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR (or Fibroblast Growth Factor Receptor) polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF (or Epidermal Growth Factor) polypeptide, a receptor of an EGF polypeptide, a neuregulin polypeptide (e.g., a neuregulin isoform I, II, III, IV, V, or VI polypeptide), a receptor of a neuregulin polypeptide, a HER (Human Epidermal Growth Factor Receptor) polypeptide (e.g., HER1, HER2, HER3, or HER4 polypeptide), an ECD of a HER polypeptide (e.g., an ECD of a HER1, a HER2, a HER3, or a HER4 polypeptide), an EpCAM (or Epithelial Cell Adhesion Molecule) polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP (or Calcitonin Gene-Related Peptide) polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL (or Calcitonin Receptor-Like) polypeptide (e.g., an ECD of a CALCRL polypeptide), a RAMP (or Receptor Activity-Modifying Protein) polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF (or Insulin-Like Growth Factor) polypeptide (e.g., an IGF-1 or an IGF-2 polypeptide), a receptor of an IGF polypeptide (e.g., a receptor of an IGF-1 or an IGF-2 polypeptide), an IGFR (or Insulin-Like Growth Factor Receptor) polypeptide (e.g., an IGFR1 or an IGFR2 polypeptide), an ECD of an IGFR polypeptide (e.g., an ECD of an IGFR1 or an IGFR2 polypeptide), an IGFBP (or Insulin-Like Growth Factor Binding Protein) polypeptide (e.g., IGFBP1, IGFBP2, IGFBP3, IGFBP4, IGFBP5, or IGFBP6 polypeptide), a VEGF (or Vascular Endothelial Growth Factor) polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF (or Placental Growth Factor) polypeptide), a VEGFR (or Vascular Endothelial Growth Factor Receptor) polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 (or FMS-like Tyrosine Kinase 1) receptor polypeptide (e.g., an ECD of an FLT1 receptor polypeptide), an IL36 (or Interleukin 36) polypeptide (e.g., IL36A, IL36B, or IL36G polypeptide), a receptor of an IL36 polypeptide (e.g., a receptor of an IL36A, an IL36B, or an IL36G polypeptide), an IL36R (or Interleukin 36 Receptor) polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 (or Sialic Acid-Binding Ig-Like Lectin 10) polypeptide, a PCSK9 (or Proprotein Convertase Subtilisin/Kexin Type 9) polypeptide, a receptor of a PCSK9 polypeptide, an LDLR (or Low Density Lipoprotein Receptor) polypeptide (e.g., an ECD of an LDLR polypeptide), a CEA (or Carcinoembryonic Antigen) polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF (or B-cell Activating Factor) polypeptide, a receptor of a BAFF polypeptide, a TRAF (or TNF Receptor Associated Factor) polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA (or B-cell Maturation Antigen) polypeptide, a SOST polypeptide, a receptor of a SOST (or Sclerostin) polypeptide, an LRP (or Low-density Lipoprotein Receptor-Related Protein) polypeptide (e.g., an LRP5 or an LRP6 polypeptide), an ECD of an LRP polypeptide (e.g., an ECD of an LRP5 or an LRP6 polypeptide), a DLL (or Delta-like) polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF (or von Willebrand Factor) polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 (or Interleukin 2) polypeptide, a receptor of an IL2 polypeptide, an IL2R (or Interleukin 2 Receptor) polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ (or Transforming Growth Factor (3) polypeptide, a receptor of a TGFβ polypeptide, a Decorin polypeptide, an EIF3I (or Eukaryotic Translation Initiation Factor 3 Subunit 1) polypeptide, a LTBP1 (or Latent-transforming Growth Factor Beta-Binding Protein 1) polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK (or Kallikrein) polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl (or Receptor Activator of Nuclear Factor Kappa-B ligand) polypeptide, a receptor of a Rankl polypeptide, a RANK (or Receptor Activator of Nuclear Factor Kappa-B) polypeptide (e.g., an ECD of a RANK polypeptide), a TSLP (or Thymic Stromal Lymphopoietin) polypeptide, a receptor of a TSLP polypeptide, a CRLF2 (or Cytokine Receptor-like Factor 2) polypeptide (e.g., an ECD of a CRLF2 polypeptide), an IL7Rα polypeptide (e.g., an ECD of an IL7Rα polypeptide), an S1P (or Specificity Protein 1) polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 (or Cytotoxic T-lymphocyte-Associated Protein 4) polypeptide (e.g., an ECD of a CTLA-4 polypeptide), a GnRH (or Gonadotropin-Releasing Hormone) polypeptide, a receptor of a GNRH polypeptide, a GnRHR (or Gonadotropin-Releasing Hormone Receptor) polypeptide (e.g., an ECD of a GnRHR polypeptide), an ICAM (or Intercellular Adhesion Molecule) polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 (or Complement component 5) polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, a glucagon polypeptide, or etc.

In some embodiments, the therapeutic polypeptide is a canine polypeptide, a feline polypeptide, or an equine polypeptide.

Exemplary antibody or antibody fragment include, but are not limited to, those that recognize one or more of the following polypeptides: a late embryogenesis abundant (LEA) polypeptide (e.g., an LEA-3 polypeptide), an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an EPO polypeptide, a somatostatin polypeptide, a GLP1 polypeptide, a glucagon polypeptide, or etc.

In some embodiments, the target polypeptide is a canine polypeptide, a feline polypeptide, or an equine polypeptide.

A “linker” refers to one or more amino acid residues that connects a first polypeptide with a second polypeptide.

In some embodiments, the linker is a flexible, non-structural linker. In some embodiments, the linker is a glycine-rich, serine-rich, or glycine- and serine-rich linker. In some embodiments, a linker comprises 100%, at least 95%, at least 90%, or at least 85% serine and/or glycine amino acid residues.

### Exemplary Expression and Production of IgG Fc Variants and Fusion Molecules

A nucleotide sequence encoding a polypeptide of interest, such as a variant IgG Fc polypeptide or other polypeptide described herein, can be inserted into an expression vector suitable for expression in a selected host cell. A variant IgG Fc polypeptide or variant IgG Fc fusion protein may be expressed by culturing a host cell transfected with an expression vector comprising the nucleotide sequence.

A “vector” is a plasmid that can be used to transfer DNA sequences from one organism to another or to express a gene of interest. A vector typically includes an origin of replication and regulatory sequences which regulate the expression of the gene of interest, and may or may not carry a selective marker gene, such as an antibiotic resistance gene. A vector is suitable for the host cell in which it is to be expressed. A vector may be termed a “recombinant vector” when the gene of interest is present in the vector.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 cells, and CHO cells, and their derivatives, such as 293-6E, DG44, CHO-S, and CHO-K cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) encoding an amino acid sequence(s) provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated.”

A “signal sequence” refers to a sequence of amino acid residues or polynucleotides encoding such, which facilitates secretion of a polypeptide of interest and is typically cleaved upon export of the polypeptide to the outside of the cell surface membrane.

In some embodiments, a variant IgG Fc polypeptide or other polypeptide described herein is isolated using chromatography, such as size exclusion chromatography, ion exchange chromatography, protein A column chromatography, hydrophobic interaction chromatography, and CHT chromatography.

A label can be attached to a variant IgG Fc polypeptide or a polypeptide comprising a variant Fc polypeptide. A “label” means a moiety attached to a molecule to render it detectable. In some embodiments, a variant IgG Fc polypeptide is labeled with a detectable moiety including but not limited to radioisotopes, fluorescent labels, and various enzyme-substrate labels known in the art. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, p-galactosidase, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety. General techniques to be used in performing the various immunoassays noted above are known to those of ordinary skill in the art.

### Exemplary Variant IgG Fc Polypeptide Affinity to FcRn

Variant IgG Fc polypeptides described herein may have altered binding affinity to FcRn, such as enhanced binding affinity to FcRn at an acidic pH (e.g., at a pH in the range of from about 5.0 to about 6.5). Variant IgG Fc polypeptides described herein may extend the half-life or improve pharmacokinetics of an antibody or variant IgG Fc fusion protein in vivo.

“Neonatal Fc receptor” or “FcRn,” as used herein, is a polypeptide comprising the entirety or a portion of FcRn that is capable of binding a wild-type IgG of the same species, with the exception of canine IgG-C. In some embodiments, an FcRn is an FcRn extracellular domain (ECD). In some embodiments, FcRn comprises the amino acid sequence of SEQ ID NO: 18, SEQ ID NO: 19, SEQ ID NO: 21, SEQ ID NO: 22, SEQ ID NO: 24, or SEQ ID NO: 25. In some embodiments, FcRn is associated with B2M in a protein complex (“FcRn/B2M protein complex” or “FcRn/B2M complex”).

An “extracellular domain” (“ECD”) is the portion of a polypeptide that extends beyond the transmembrane domain into the extracellular space. The term “extracellular domain,” as used herein, may comprise a complete extracellular domain or may comprise a truncated extracellular domain missing one or more amino acids, that binds to its ligand. The composition of the extracellular domain may depend on the algorithm used to determine which amino acids are in the membrane. Different algorithms may predict, and different systems may express, different extracellular domains for a given protein.

“Beta-2-microglobulin” or “B2M,” as used herein, is a polypeptide comprising the entirety or a portion of B2M that is capable of associating with FcRn. In some embodiments, B2M comprises the amino acid sequence of SEQ ID NO: 20, SEQ ID NO: 23, or SEQ ID NO: 26.

The term “binds” to a substance is a term that is well understood in the art, and methods to determine such binding are also well known in the art. A molecule is said to exhibit “binding” if it reacts, associates with, or has affinity for a particular cell or substance and the reaction, association, or affinity is detectable by one or more protein-protein interaction tools known in the art, such as, for example, immunoblot, ELISA, KinEx A, biolayer interferometry (BLI), surface plasmon resonance (SPR) devices, or etc.

The term “affinity” means the strength of the sum total of noncovalent interactions between a single binding site of a molecule (for example, a receptor) and its binding partner (for example, a ligand). The affinity of a molecule X for its partner Y can generally be represented by the dissociation constant (K_{D}). Affinity can be measured by common protein-protein interaction tools known in the art, such as, for example, immunoblot, ELISA, KinEx A, biolayer interferometry (BLI), or surface plasmon resonance (SPR) devices.

“Surface plasmon resonance” denotes an optical phenomenon that allows for the analysis of real-time biospecific interactions by detection of alterations in protein concentrations within a biosensor matrix, for example using the BIAcore™ system (BlAcore International AB, a GE Healthcare company, Uppsala, Sweden and Piscataway, N.J.). For further descriptions, see Jonsson et al. (1993) Ann. Biol. Clin. 51: 19-26.

“Biolayer interferometry” refers to an optical analytical technique that analyzes the interference pattern of light reflected from a layer of immobilized protein on a biosensor tip and an internal reference layer. Changes in the number of molecules bound to the biosensor tip cause shifts in the interference pattern that can be measured in real-time. A nonlimiting exemplary device for biolayer interferometry is an Octet® system (Pall ForteBio LLC). See, e.g., Abdiche et al., 2008, Anal. Biochem. 377: 209-277.

The terms “K_{D},” “K_{d},” “Kd” or “Kd value” as used interchangeably to refer to the equilibrium dissociation constant of a receptor-ligand interaction or antibody-antigen interaction.

In some embodiments, a variant IgG Fc polypeptide binds to FcRn with a dissociation constant (K_{D}) of less than 5×10⁻⁶ M, less than 1×10⁻⁶ M, less than 5×10⁻⁶ M, less than 1×10⁻⁷ M, less than 5×10⁻⁸ M, less than 1×10⁻⁸ M, less than 5×10⁻⁹ M, less than 1×10⁻⁹ M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry.

In some embodiments, a polypeptide comprises a variant IgG Fc polypeptide having an increased serum half-life relative to a polypeptide comprising a wild-type Fc polypeptide.

In some embodiments, the KD of an Fc polypeptide, such as a variant IgG Fc polypeptide, to FcRn or a FcRn/B2M protein complex is measured by using biolayer interferometry assays using a biosensor, such as an Octet® System (Pall ForteBio LLC, Fremont, Calif.) according to the supplier's instructions. In brief, biotinylated FcRn or FcRn/B2M protein complex is bound to the sensor tip and the association of Fc polypeptide is monitored for a specified time or until steady state is reached. Dissociation may be monitored for a specified time or until steady state is reached. A buffer only blank curve is subtracted to correct for any drift. The data are fit to a 2:1 binding model using ForteBio data analysis software to determine association rate constant (kₒₙ), dissociation rate constant (k_{off}), and the K_{d}. The equilibrium dissociation constant (K_{D}) is calculated as the ratio of k_{off}/kₒₙ. The term “kₒₙ” refers to the rate constant for association of a molecule X to its partner Y and the term “k_{off}” refers to the rate constant for dissociation of a molecule X or partner Y from the molecule X/partner Y complex.

In some embodiments, association of an Fc polypeptide, such as a variant IgG Fc polypeptide, with FcRn or FcRn/B2M protein complex may be tested at various concentrations, such as a concentration less than about 0.5 μg/mL, a concentration in the range of about 0.5 μg/mL to about 100 μg/mL, a concentration in the range of about 0.5 μg/mL to about 10 μg/mL, a concentration in the range of about 10 μg/mL to about 100 μg/mL, or a concentration in the range of about 5 μg/mL to about 50 μg/mL. In some embodiments, the concentration of Fc polypeptide tested for association with FcRn or FcRn/B2M protein complex is about 0.5 μg/mL, about 1 μg/mL, about 5 μg/mL, about 10 μg/mL, about 15 μg/mL, about 20 μg/mL, about 25 μg/mL, about 30 μg/mL, about 40 μg/mL, about 50 μg/mL, about 60 μg/mL, about 70 μg/mL, about 75 μg/mL, about 80 μg/mL, about 90 μg/mL, or about 100 μg/mL.

In some embodiments, association or dissociation of an Fc polypeptide, such as a variant IgG Fc polypeptide, with FcRn or an FcRn/B2M protein complex may be tested at various pHs. In some embodiments, association or dissociation of an Fc polypeptide with FcRn or an FcRn/B2M protein complex is tested at an acidic pH. In some embodiments, association or dissociation of an Fc polypeptide with FcRn or an FcRn/B2M protein complex is tested at a neutral pH. In some embodiments, association or dissociation of an Fc polypeptide with FcRn or an FcRn/B2M protein complex is tested at a pH in the range of about 5.0 to about 6.5. In some embodiments, association or dissociation of an Fc polypeptide with FcRn or an FcRn/B2M protein complex is tested at a pH of about 5.0, about 6.0, about 6.5, or about 7.0. In some embodiments, association or dissociation of an Fc polypeptide with FcRn or an FcRn/B2M protein complex is tested at a pH of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

Buffers for dilutions and binding steps may be adjusted to the various pHs described above and are known to those of ordinary skill in the art. For example, a buffer of 20 mM MES and 15 mM NaCl adjusted to a pH in the range of about 5.0 to about 6.5 may be used in the binding assay. As another example, phosphate buffered saline, pH 7.4 may be used in the binding assay. As a further example, a buffer of 20 mM phosphate, 150 mM NaCl, pH 7.2 may be used in the binding assay.

In some embodiments, association or dissociation of an Fc polypeptide, such as a variant IgG Fc polypeptide, with FcRn or an FcRn/B2M protein complex is monitored for a specified time or until steady state is reached. In some embodiments, association or dissociation of an Fc polypeptide with FcRn or an FcRn/B2M protein complex is monitored for 30 seconds, 60 seconds, 90 seconds, 120 seconds, 150 seconds, 180 seconds, 210 seconds, 240 seconds, 270 seconds, 300 seconds, 330 seconds, 360 seconds, 390 seconds, 420 seconds, 450 seconds, 480 seconds, 510 seconds, 540 seconds, 570 seconds, 600 seconds, 630 seconds, 660 seconds, 690 seconds, 720 seconds, 750 seconds, 780 seconds, 810 seconds, 840 seconds, 870 seconds, or 900 seconds.

In some embodiments, a pharmacokinetic analysis is performed to determine any number of pharmacokinetic parameters including Tmax, Cmax, and Area under the Curve (AUC). For example, an animal may be administered a polypeptide described herein and serum samples collected at different time intervals (e.g., pre-injection and/or at 0.5, 1, 6, 24, 48, 72, 168, 216, and/or 336 hours post administration). The polypeptide concentrations in the serum samples may be determined, for example by ELISA.“Increased” or “greater” means an increase relative to a reference. In some embodiments, by “increased” or “greater” is meant the ability to cause an overall increase of about 5% or more, of about 10% or more, of about 20% or more, of about 30% or more, of about 40% or more, of about 50% or more, of about 60% or more, of about 70% or more, of about 80% or more, of about 90% or more, of about 100% or more, of about 125% or more, of about 150% or more, of about 200% or more relative to a reference value. In some embodiments, by “increase” or “greater” is meant the ability to cause an overall increase of about 5% to about 50%, of about 10% to about 20%, of about 50% to about 100%, of about 25% to about 70% relative to a reference value.

In some embodiments, a variant Fc polypeptide, such as a variant IgG Fc polypeptide, is capable of binding to FcRn or FcRn/B2M with an increased affinity of about 5% or more, of about 10% or more, of about 20% or more, of about 30% or more, of about 40% or more, of about 50% or more, of about 60% or more, of about 70% or more, of about 80% or more, of about 90% or more, of about 100% or more, of about 125% or more, of about 150% or more, of about 200% or more relative to a reference Fc polypeptide. In some embodiments, a variant Fc polypeptide is capable of binding to FcRn or FcRn/B2M with an increased affinity of about 5% to about 50%, of about 10% to about 20%, of about 50% to about 100%, of about 25% to about 70% relative to a reference Fc polypeptide. In some embodiments, the reference Fc polypeptide is a wild-type Fc polypeptide. In some embodiments, the Fc polypeptide is a different variant Fc polypeptide. In some embodiments, the affinity is measured by biolayer interferometry at a pH in the range of from about 5.0 to about 6.5.

### Exemplary Pharmaceutical Compositions

The terms “pharmaceutical formulation” and “pharmaceutical composition” refer to a preparation which is in such form as to permit the biological activity of the active ingredient(s) to be effective, and which contains no additional components that are unacceptably toxic to a subject to which the formulation would be administered.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The pharmaceutical composition can be stored in lyophilized form. Thus, in some embodiments, the preparation process includes a lyophilization step. The lyophilized composition may then be reformulated, typically as an aqueous composition suitable for parenteral administration, prior to administration to the dog, cat, or horse. In other embodiments, particularly where a variant IgG Fc polypeptide or other polypeptide described herein is highly stable to thermal and oxidative denaturation, the pharmaceutical composition can be stored as a liquid, i.e., as an aqueous composition, which may be administered directly, or with appropriate dilution, to the dog, cat, or horse. A lyophilized composition can be reconstituted with sterile Water for Injection (WFI). Bacteriostatic reagents, such benzyl alcohol, may be included. Thus, the invention provides pharmaceutical compositions in solid or liquid form.

The pH of the pharmaceutical compositions may be in the range of from about pH 5 to about pH 8, when administered. The compositions of the invention are sterile if they are to be used for therapeutic purposes. Sterility can be achieved by any of several means known in the art, including by filtration through sterile filtration membranes (e.g., 0.2 micron membranes). Sterility may be maintained with or without anti-bacterial agents.

### Exemplary Uses of Fc Polypeptides and Pharmaceutical Compositions

A polypeptide comprising a variant Fc polypeptide, such as a variant IgG Fc polypeptide, of the invention or pharmaceutical compositions comprising a variant Fc polypeptide of the invention may be useful for extending product half-life in vivo in a companion animal, including, but not limited to, canine, feline, or equine.

As used herein, “treatment” is an approach for obtaining beneficial or desired clinical results. “Treatment” as used herein, covers any administration or application of a therapeutic for disease in a mammal, including a companion animal. For purposes of this disclosure, beneficial or desired clinical results include, but are not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

A “therapeutically effective amount” of a substance/molecule, agonist or antagonist may vary according to factors such as the type of disease to be treated, the disease state, the severity and course of the disease, the type of therapeutic purpose, any previous therapy, the clinical history, the response to prior treatment, the discretion of the attending veterinarian, age, sex, and weight of the animal, and the ability of the substance/molecule, agonist or antagonist to elicit a desired response in the animal. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic or prophylactic result.

In some embodiments, a variant Fc polypeptide (such as a variant IgG Fc polypeptide), a variant Fc fusion molecule (such as a variant IgG Fc fusion protein), or pharmaceutical composition comprising a variant Fc polypeptide or variant Fc fusion molecule is administered parenterally, by subcutaneous administration, intravenous infusion, or intramuscular injection. In some embodiments, a variant Fc polypeptide fusion molecule, or pharmaceutical composition comprising a variant Fc polypeptide fusion molecule is administered as a bolus injection or by continuous infusion over a period of time. In some embodiments, a variant Fc polypeptide fusion molecule, or pharmaceutical composition comprising a variant Fc polypeptide fusion molecule is administered by an intramuscular, an intraperitoneal, an intracerebrospinal, a subcutaneous, an intra-arterial, an intrasynovial, an intrathecal, or an inhalation route.

In some embodiments, a variant Fc polypeptide fusion molecule described herein is administered in an amount in the range of 0.0001 mg/kg body weight to 100 mg/kg body weight per dose. In some embodiments, a variant Fc polypeptide fusion molecule described herein is administered to a companion animal at one time or over a series of treatments.

In some embodiments, a variant IgG Fc polypeptide or other polypeptide described herein, or a pharmaceutical composition comprising such is administered to a companion animal at one time or over a series of treatments. In some embodiments, the dose is administered once per week for at least two or three consecutive weeks, and in some embodiments, this cycle of treatment is repeated two or more times, optionally interspersed with one or more weeks of no treatment. In other embodiments, the therapeutically effective dose is administered once per day for two to five consecutive days, and in some embodiments, this cycle of treatment is repeated two or more times, optionally interspersed with one or more days or weeks of no treatment.

Administration “in combination with” one or more further therapeutic agents includes simultaneous (concurrent) and consecutive or sequential administration in any order. The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about a specified number of minutes. The term “sequentially” is used herein to refer to administration of two or more therapeutic agents where the administration of one or more agent(s) continues after discontinuing the administration of one or more other agent(s), or wherein administration of one or more agent(s) begins before the administration of one or more other agent(s). For example, administration of the two or more therapeutic agents are administered with a time separation of more than about a specified number of minutes. As used herein, “in conjunction with” refers to administration of one treatment modality in addition to another treatment modality. As such, “in conjunction with” refers to administration of one treatment modality before, during or after administration of the other treatment modality to the animal.

The following examples illustrate particular aspects of the disclosure and are not intended in any way to limit the disclosure.

### EXAMPLES

### Example 1

### Expression and Purification of Canine FcRn and Beta-2-M

A nucleotide sequence encoding canine FcRn ECD protein with poly-His tag on the C-terminal end (SEQ ID NO: 18) was synthesized and cloned into a mammalian expression vector. A nucleotide sequence encoding canine Beta-2-Microglobulin (B2M) protein (SEQ ID NO: 20) was synthesized and cloned into a mammalian expression vector. Both vectors were cotransfected to 293 cells or CHOS. The supernatant containing canine FcRn/B2M protein complex was collected and filtered. Canine FcRn/B2M protein complex was affinity purified using Ni-NTA column (CaptivA® Protein A Affinity Resin, Repligen). Feline and equine FcRn/B2M protein complexes may be produced using a similar method.

### Example 2

### Variant Canine IgG-A Fc Polypeptides

Variants of canine IgG-A Fc polypeptide (e.g., variants of SEQ ID NO: 1) may be prepared having one or more of the amino acid substitutions listed in Table 3 at a position corresponding to the listed position of SEQ ID NO: 1.

TABLE 3
Canine IgG-A Fc Amino Acid Substitutions

| Original Residue (position) | Substitution(s) |
|---|---|
| Gly (9) | Tyr |
| Lys (18) | Pro |
| Ile (22) | His; Ile; or Pro |
| Leu (23) | Val; Arg; or Lys |
| Arg (24) | Phe; Tyr; Met; or Val |
| Ile (25) | Thr |
| Thr (26) | Trp; Tyr; His; Leu; Phe; Val; or Met |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Lys (60) | Ala |
| Pro (79) | Gln; Arg; Ala; Glu: or Met |
| Ile (80) | His or Phe |
| Gln (83) | Ala; Tyr; or Phe |
| Ser (132) | Ile |
| Asp (148) | Val |
| Pro (162) | Leu; Phe; or Tyr |
| Ser (183) | Ile |
| Met (203) | Leu or Trp |
| Glu (205) | Pro |
| Asn (209) | Ser; Ala; Arg; Phe; Tyr; His; or Trp |

The binding of any of the variant canine IgG-A Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type canine IgG-A Fc polypeptide, another wild-type or variant canine IgG Fc polypeptide, or a wild-type or variant IgG Fc polypeptide of another companion animal, etc.).

Binding analysis may be performed using a biosensor Octet. Briefly, FcRn/B2M complex (e.g., a FcRn/B2M protein complex produced by the method of Example 1) may be biotinylated and free unreacted biotin removed (e.g., by dialysis). Biotinylated FcRn/B2M complex is captured on streptavidin sensor tips. Association of FcRn/B2M complex with various concentrations (e.g., 10 μg/mL) of variant IgG Fc polypeptides is monitored for a specified time or until steady state is reached. Dissociation is monitored for a specified time or until steady state is reached. A buffer only blank curve may be subtracted to correct for any drift. The data are fit to a 1:1 binding model using ForteBio™ data analysis software to determine the kon, koff, and the Kd. The binding assay (either association or dissociation steps) may be performed at different pH conditions using buffers. For example, the association step and/or dissociation step may be performed at a pH in the range of from about 5.0 to about 6.5 (e.g., 20 mM MES and 15 mM NaCl), or at a pH of about 7.4 (e.g., PBS).

### Example 3

### Variant Canine IgG-B Fc Polypeptides

Variants of canine IgG-B Fc polypeptide (e.g., variants of SEQ ID NO: 2) may be prepared having one or more of the amino acid substitutions listed in Table 4 at a position corresponding to the listed position of SEQ ID NO: 2.

TABLE 4
Canine IgG-B Fc Amino Acid Substitutions

| Original Residue (position) | Substitution(s) |
|---|---|
| Gly (9) | Tyr |
| Lys (18) | Pro |
| Thr (22) | His; Ile; or Pro |
| Leu (23) | Val; Arg; or Lys |
| Leu (24) | Phe; Tyr; Met; Arg; or Val |
| Ile (25) | Thr |
| Ala (26) | Trp; Tyr; His; Leu; Phe; Val; Met, Thr; or |
| | Ser |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Lys (60) | Ala |
| Pro (79) | Gln; Arg; Ala; Glu; or Met |
| Ile (80) | His or Phe |
| Gly (81) | Glu |
| Gln (83) | Ala; Tyr; or Phe |
| Lys (132) | Ile |
| Asp (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Glu (213) | Ile or Val |

The binding of any of the variant canine IgG-B Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type canine IgG-B Fc polypeptide, another wild-type or variant canine IgG Fc polypeptide, or a wild-type or variant IgG Fc polypeptide of another companion animal, etc.). The binding assay described in Example 2 may be used.

### Example 4

### Variant Canine IgG-C Fc Polypeptides

Variants of canine IgG-C Fc (e.g., variants of SEQ ID NO: 3) may be prepared having one or more of the amino acid substitutions listed in Table 5 at a position corresponding to the listed position of SEQ ID NO: 3.

TABLE 5
Canine IgG-C Fc Amino Acid Substitutions

| Original Residue (position) | Substitution(s) |
|---|---|
| Gly (9) | Tyr |
| Lys (18) | Pro |
| Ile (22) | His; Pro |
| Leu (23) | Val; Arg; or Lys |
| Val (24) | Phe; Tyr; Met; or Arg |
| Ala (26) | Trp; Tyr; His; Leu; Phe; Val; Met, Thr; or |
| | Ser |
| Arg (27) | Leu; Met; Pro; Ile; Asp; or Trp |
| Thr (28) | Met; Glu; Asp; His; Ile; or Val |
| Thr (30) | Met or Val |
| Val (31) | Ile |
| Leu (60) | Ala |
| Pro (79) | Gln; Arg; Ala; Glu; or Met |
| Ile (80) | His or Phe |
| Gly (81) | Glu |
| Gln (83) | Ala; Tyr; or Phe |
| Lys (132) | Ile |
| Glu (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |

The binding of any of the variant canine IgG-C Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type canine IgG-C Fc polypeptide, another wild-type or variant canine IgG Fc polypeptide, or a wild-type or variant IgG Fc polypeptide of another companion animal, etc.). The binding assay described in Example 2 may be used.

### Example 5

### Variant Canine IgG-D Fc Polypeptides

Variants of canine IgG-D Fc (e.g., variants of SEQ ID NO: 4) may be prepared having one or more of the amino acid substitutions listed in Table 6 at a position corresponding to the listed position of SEQ ID NO: 4.

TABLE 6
Canine IgG-D Fc Amino Acid Substitutions

| Original Residue (position) | Substitution(s) |
|---|---|
| Gly (9) | Tyr |
| Lys (18) | Pro |
| Ile (22) | His or Pro |
| Leu (23) | Val; Arg; or Lys |
| Arg (24) | Phe; Tyr; Met; or Val |
| Ile (25) | Thr |
| Thr (26) | Trp; Tyr; His; Leu; Phe; Val; or Met |
| Arg (27) | Leu; Met; Pro; Ile; Asp; or Trp |
| Thr (28) | Met; Glu; Asp; His; Ile; or Val |
| Glu (30) | Met or Val |
| Lys (60) | Ala |
| Pro (79) | Gln; Arg; Ala; Glu; or Met |
| Ile (80) | His or Phe |
| Gln (83) | Ala; Tyr; or Phe |
| Ser (132) | Ile |
| Glu (149) | Val |
| Glu (163) | Leu; Phe; or Tyr |
| Ser (183) | Ile |
| Met (203) | Leu or Trp |
| Glu (205) | Pro |
| Asn (209) | Ser; Ala; Arg; Phe; Tyr; His; or Trp |

The binding of any of the variant canine IgG-D Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type canine IgG-D Fc polypeptide, another wild-type or variant canine IgG Fc polypeptide, or a wild-type or variant IgG Fc polypeptide of another companion animal, etc.). The binding assay described in Example 2 may be used.

### Example 6

### Variant Equine IgG-1 Fc Polypeptides

Variants of equine IgG-1 Fc (e.g., variants of SEQ ID NO: 5) may be prepared having one or more of the amino acid substitutions listed in Table 7 at a position corresponding to the listed position of SEQ ID NO: 5.

TABLE 7
Equine IgG-1 Fc Amino Acid Substitutions

| Original Residue (position) | Substitution(s) |
|---|---|
| Gly (9) | Tyr |
| Asn (18) | Pro |
| Thr (22) | His; Ile; or Pro |
| Leu (23) | Val; Arg; or Lys |
| Met (24) | Phe; Tyr; Met; Arg; or Val |
| Ile (25) | Thr |
| Thr (26) | Trp; Tyr; His; Leu; Phe; Val; or Met, |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Arg (79) | Gln; Glu; or Met |
| Ile (80) | His or Phe |
| Gln (81) | Glu |
| Gln (83) | Ala; Tyr; or Phe |
| Lys (132) | Ile |
| Glu (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Lys (213) | Ile or Val |

The binding of any of the variant equine IgG-1 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-1 Fc polypeptide, another wild-type or variant equine IgG Fc polypeptide, or a wild-type or variant IgG Fc polypeptide of another companion animal, etc.). The binding assay described in Example 2 may be used.

### Example 7

### Variant Equine IgG-2 Fc Polypeptides

Variants of equine IgG-2 Fc (e.g., variants of SEQ ID NO: 6) may be prepared having one or more of the amino acid substitutions listed in Table 8 at a position corresponding to the listed position of SEQ ID NO: 6.

TABLE 8
Equine IgG-2 Fc Amino Acid Substitutions

| Original Residue (position) | Substitution(s) |
|---|---|
| Gly (9) | Tyr |
| Asn (18) | Pro |
| Ala (22) | His; Ile; or Pro |
| Leu (23) | Val; Arg; or Lys |
| Met (24) | Phe; Tyr; Met; Arg; or Val |
| Ile (25) | Thr |
| Ser (26) | Trp; Tyr; His; Leu; Phe; Val; Met; or Thr |
| Arg (27) | Leu; Met; Pro; Ile; Asp; or Trp |
| Thr (28) | Met; Glu; Asp; His; Ile; or Val |
| Pro (79) | Gln; Arg; Ala; Glu; or Met |
| Ile (80) | His or Phe |
| Gln (81) | Glu |
| Gln (83) | Ala; Tyr; or Phe |
| Lys (132) | Ile |
| Asp (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Thr (213) | Ile or Val |

The binding of any of the variant equine IgG-2 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-2 Fc polypeptide, another wild-type or variant equine IgG Fc polypeptide, or a wild-type or variant IgG Fc polypeptide of another companion animal, etc.). The binding assay described in Example 2 may be used.

### Example 8

### Variant Equine IgG-3 Fc Polypeptides

Variants of equine IgG-3 Fc (e.g., variants of SEQ ID NO: 7) may be prepared having one or more of the amino acid substitutions listed in Table 9 at a position corresponding to the listed position of SEQ ID NO: 7.

TABLE 9
Equine IgG-3 Fc Amino Acid Substitutions

| Original Residue (position) | Substitution(s) |
|---|---|
| Gly (9) | Tyr |
| Lys (18) | Pro |
| Val (22) | His; Ile; or Pro |
| Leu (23) | Val; Arg; or Lys |
| Met (24) | Phe; Tyr; Met; Arg; or Val |
| Ile (25) | Thr |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Lys (60) | Ala |
| Arg (79) | Gln; Glu; or Met |
| Ile (80) | His or Phe |
| Gln (81) | Glu |
| Gln (83) | Ala; Tyr; or Phe |
| Lys (132) | Ile |
| Asp (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Lys (213) | Ile or Val |

The binding of any of the variant equine IgG-3 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-3 Fc polypeptide, another wild-type or variant equine IgG Fc polypeptide, or a wild-type or variant IgG Fc polypeptide of another companion animal, etc.). The binding assay described in Example 2 may be used.

### Example 9

### Variant Equine IgG-4 Fc Polypeptides

Variants of equine IgG-4 Fc (e.g., variants of SEQ ID NO: 8) may be prepared having one or more of the amino acid substitutions listed in Table 10 at a position corresponding to the listed position of SEQ ID NO: 8.

TABLE 10
Equine IgG-4 Fc Amino Acid Substitutions

| Original Residue (position) | Substitution(s) |
|---|---|
| Val (9) | Tyr |
| Lys (18) | Pro |
| Val (22) | His; Ile; or Pro |
| Leu (23) | Val; Arg; or Lys |
| Met (24) | Phe; Tyr; Met; Arg; or Val |
| Ile (25) | Thr |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Pro (79) | Gln; Arg; Ala; Glu; or Met |
| Ile (80) | His or Phe |
| Lys (83) | Ala; Tyr; or Phe |
| Lys (132) | Ile |
| Asp (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Lys (213) | Ile or Val |

The binding of any of the variant equine IgG-4 Fc polypeptide to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-4 Fc polypeptide, another wild-type or variant equine IgG Fc polypeptide, or a wild-type or variant IgG Fc polypeptide of another companion animal, etc.). The binding assay described in Example 2 may be used.

### Example 10

### Variant Equine IgG-5 Fc Polypeptides

Variants of equine IgG-5 Fc (e.g., variants of SEQ ID NO: 9) may be prepared having one or more of the amino acid substitutions listed in Table 11 at a position corresponding to the listed position of SEQ ID NO: 9.

TABLE 11
Equine IgG-5 Fc Amino Acid Substitutions

| Original Residue (position) | Substitution(s) |
|---|---|
| Gly (9) | Tyr |
| Lys (18) | Pro |
| Val (22) | His; Ile; or Pro |
| Leu (23) | Val; Arg; or Lys |
| Lys (24) | Phe; Tyr; Met; Arg; or Val |
| Ile (25) | Thr |
| Arg (27) | Leu; Met; Pro; Ile; Asp; or Trp |
| Lys (28) | Met; Glu; Asp; His; Ile; or Val |
| Glu (30) | Met or Val |
| Val (31) | Ile |
| Pro (79) | Gln; Arg; Ala; Glu; or Met |
| Ile (80) | His or Phe |
| Gln (81) | Glu |
| Gln (83) | Ala; Tyr; or Phe |
| Lys (132) | Ile |
| Glu (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Lys (213) | Ile or Val |

The binding of any of the variant equine IgG-5 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-5 Fc polypeptide, another wild-type or variant equine IgG Fc polypeptide, or a wild-type or variant IgG Fc polypeptide of another companion animal, etc.). The binding assay described in Example 2 may be used.

### Example 11

### Variant Equine IgG-6 Fc Polypeptides

Variants of equine IgG-6 Fc (e.g., variants of SEQ ID NO: 10) may be prepared having one or more of the amino acid substitutions listed in Table 12 at a position corresponding to the listed position of SEQ ID NO: 10.

TABLE 12
Equine IgG-6 Fc Amino Acid Substitutions

| Original Residue (position) | Substitution(s) |
|---|---|
| Asn (18) | Pro |
| Thr (22) | His; Ile; or Pro |
| Leu (23) | Val; Arg; or Lys |
| Met (24) | Phe; Tyr; Arg; or Val |
| Ile (25) | Thr |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Pro (79) | Gln; Arg; Ala; Glu; Met |
| Ile (80) | His or Phe |
| Gln (81) | Glu |
| Gln (83) | Ala; Tyr; Phe |
| Lys (132) | Ile |
| Asp (148) | Val |
| Glu (162) | Leu; Phe; or Tyr |
| Ser (182) | Ile |
| Met (202) | Leu or Trp |
| Glu (204) | Pro |
| His (208) | Ser; Ala; Arg; Phe; Tyr; or Trp |
| Lys (213) | Ile or Val |

The binding of any of the variant equine IgG-6 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-6 Fc polypeptide, another wild-type or variant equine IgG Fc polypeptide, or a wild-type or variant IgG Fc polypeptide of another companion animal, etc.). The binding assay described in Example 2 may be used.

### Example 12

### Variant Equine IgG-7 Fc Polypeptides

Variants of equine IgG-7 Fc (e.g., variants of SEQ ID NO: 11) may be prepared having one or more of the amino acid substitutions listed in Table 13 at a position corresponding to the listed position of SEQ ID NO: 11.

TABLE 13
Equine IgG-7 Fc Amino Acid Substitutions

| Original Residue (position) | Substitution(s) |
|---|---|
| Lys (18) | Pro |
| Val (22) | His; Ile; or Pro |
| Leu (23) | Val; Arg; or Lys |
| Met (24) | Phe; Tyr; Arg; or Val |
| Ile (25) | Thr |
| Arg (27) | Leu; Met; Pro; Ile; Asp; or Trp |
| Thr (28) | Met; Glu; Asp; His; Ile; or Val |
| Thr (30) | Met or Val |
| Val (31) | Ile |
| Ala (79) | Gln; Arg; Glu; or Met |
| Ile (80) | His or Phe |
| Gln (81) | Glu |
| Lys (83) | Ala; Tyr; or Phe |
| Lys (132) | Ile |
| Asp (148) | Val |
| Glu (162) | Leu; Phe; or Tyr |
| Ser (182) | Ile |
| Met (202) | Leu or Trp |
| Glu (204) | Pro |
| Asn (208) | Ser; Ala; Arg; Phe; Tyr; His; or Trp |
| Lys (213) | Ile or Val |

The binding of any of the variant equine IgG-7 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type equine IgG-7 Fc polypeptide, another wild-type or variant equine IgG Fc polypeptide, or a wild-type or variant IgG Fc polypeptide of another companion animal, etc.). The binding assay described in Example 2 may be used.

### Example 13

### Variant Feline IgG1a Fc Polypeptides

Variants of feline IgG1a Fc (e.g., variants of SEQ ID NO: 12) may be prepared having one or more of the amino acid substitutions listed in Table 14 at a position corresponding to the listed position of SEQ ID NO: 12.

TABLE 14
Feline IgG1a Fc Amino Acid Substitutions

| Original Residue (position) | Substitution(s) |
|---|---|
| Gly (9) | Tyr |
| Lys (18) | Pro |
| Thr (22) | His; Ile; or Pro |
| Leu (23) | Val; Arg; or Lys |
| Ser (24) | Phe; Tyr; Met; Arg; or Val |
| Ile (25) | Thr |
| Ser (26) | Trp; Tyr; His; Leu; Phe; Val; or Met |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Lys (60) | Ala |
| Pro (79) | Gln; Arg; Ala; Glu; or Met |
| Ile (80) | His or Phe |
| Leu (81) | Glu |
| Gln (83) | Ala; Tyr; or Phe |
| Asp (148) | Val |
| Glu (162) | Leu; Phe; or Tyr |
| Ser (182) | Ile |
| Ser (202) | Leu or Trp |
| Glu (204) | Pro |
| Ser (208) | Ala; Arg; Phe; Tyr; His; or Trp |
| Lys (213) | Ile or Val |

The binding of any of the variant feline IgG1a Fc polypeptides to FcRn/B2M (e.g., at a pH in the range of from about 5.0 to about 6.5) may be determined and compared to the binding of another IgG Fc to FcRn (e.g., the corresponding wild-type feline IgG1a Fc polypeptide, another wild-type or variant feline IgG Fc polypeptide, or a wild-type or variant IgG Fc polypeptide of another companion animal, etc.). The binding assay described in Example 2 may be used.

### Example 14

### Variant Feline IgG1b Fc Polypeptides

Variants of feline IgG1b Fc (e.g., variants of SEQ ID NO: 13) may be prepared having one or more of the amino acid substitutions listed in Table 15 at a position corresponding to the listed position of SEQ ID NO: 13.

TABLE 15
Feline IgG1b Fc Amino Acid Substitutions

| Original Residue (position) | Substitution(s) |
|---|---|
| Gly (9) | Tyr |
| Lys (18) | Pro |
| Thr (22) | His; Ile; or Pro |
| Leu (23) | Val; Arg; or Lys |
| Ser (24) | Phe; Tyr; Met; Arg; or Val |
| Ile (25) | Thr |
| Ser (26) | Trp; Tyr; His; Leu; Phe; Val; or Met |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Lys (60) | Ala |
| Pro (79) | Gln; Arg; Ala; Glu; or Met |
| Ile (80) | His or Phe |
| Leu (81) | Glu |
| Gln (83) | Ala; Tyr; or Phe |
| Arg (132) | Ile |
| Asp (148) | Val |
| Glu (162) | Leu; Phe; or Tyr |
| Ser (182) | Ile |
| Ser (202) | Leu or Trp |
| Glu (204) | Pro |
| Ser (208) | Ala; Arg; Phe; Tyr; His; or Trp |
| Lys (213) | Ile or Val |

The binding of any of the variant feline IgG1b Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type feline IgG1b Fc polypeptide, another wild-type or variant feline IgG Fc polypeptide, or a wild-type or variant IgG Fc polypeptide of another companion animal, etc.). The binding assay described in Example 2 may be used.

### Example 15

### Variant Feline IgG2 Fc Polypeptides

Variants of feline IgG2 Fc (e.g., variants of SEQ ID NO: 54) may be prepared having one or more of the amino acid substitutions listed in Table 16 at a position corresponding to the listed position of SEQ ID NO: 54.

TABLE 16
Feline IgG2 Fc Amino Acid Substitutions

| Original Residue (position) | Substitution(s) |
|---|---|
| Ala (9) | Tyr |
| Lys (18) | Pro |
| Thr (22) | His; Ile; or Pro |
| Leu (23) | Val; Arg; or Lys |
| Ser (24) | Phe; Tyr; Met; Arg; or Val |
| Ile (25) | Thr |
| Ser (26) | Trp; Tyr; His; Leu; Phe; Val; or Met |
| Arg (27) | Leu; Met; Pro; Ile; Asp; or Trp |
| Thr (28) | Met; Glu; Asp; His; Ile; or Val |
| Glu (30) | Met or Val |
| Val (31) | Ile |
| Lys (60) | Ala |
| Pro (79) | Gln; Arg; Ala; Glu; or Met |
| Ile (80) | His or Phe |
| Leu (81) | Glu |
| Gln (83) | Ala; Tyr; or Phe |
| Glu (132) | Ile |
| Asp (148) | Val |
| Glu (162) | Leu; Phe; or Tyr |
| Ser (182) | Ile |
| Ser (202) | Leu or Trp |
| Glu (204) | Pro |
| Ser (208) | Ala; Arg; Phe; Tyr; His; or Trp |
| Lys (213) | Ile or Val |

The binding of any of the variant feline IgG2 Fc polypeptides to FcRn/B2M complex (e.g., at a pH in the range of from about 5.0 to about 6.5) may be determined and compared to the binding of another IgG Fc polypeptide to FcRn/B2M complex (e.g., the corresponding wild-type feline IgG2 Fc polypeptide, another wild-type or variant feline IgG Fc polypeptide, or a wild-type or variant IgG Fc polypeptide of another companion animal, etc.). The binding assay described in Example 2 may be used.

### Example 16

### Screening Variant Canine IgG-B Polypeptides with Enhanced Canine FcRn/B2M Binding at Acidic pH

Canine FcRn with a poly-His tag (SEQ ID NO: 18) and canine B2M (SEQ ID NO: 20) heterodimer complex was transiently expressed in HEK cells and purified using Ni-NTA chromatography.

Fast Screening for Expression, Biophysical Properties and Affinity (FASEBA) of canine IgG-B Fc phage libraries was performed. Briefly, the open reading frame of canine IgG-B Fc polypeptide was subcloned into plasmid pFASEBA. Based on three-dimensional protein modeling of the canine IgG-B/canine FcRn/canine B2M complex, twelve amino acid positions of canine IgG-B were identified as being potentially involved in the binding between IgG-B and FcRn/B2M. The twelve positions of canine IgG-B identified were Thr(22), Leu(23), Leu(24), Ile(25), Ala(26), Thr (28), Gly (81), His (82), Gln (83), Leu (86), Met (202), and Asn (208) of SEQ ID NO: 2.

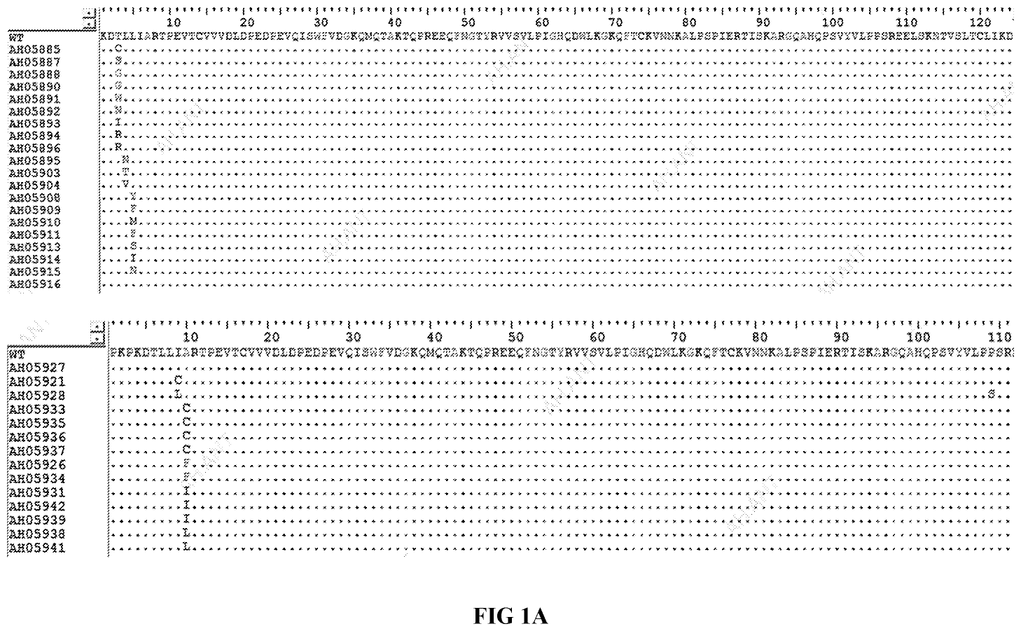

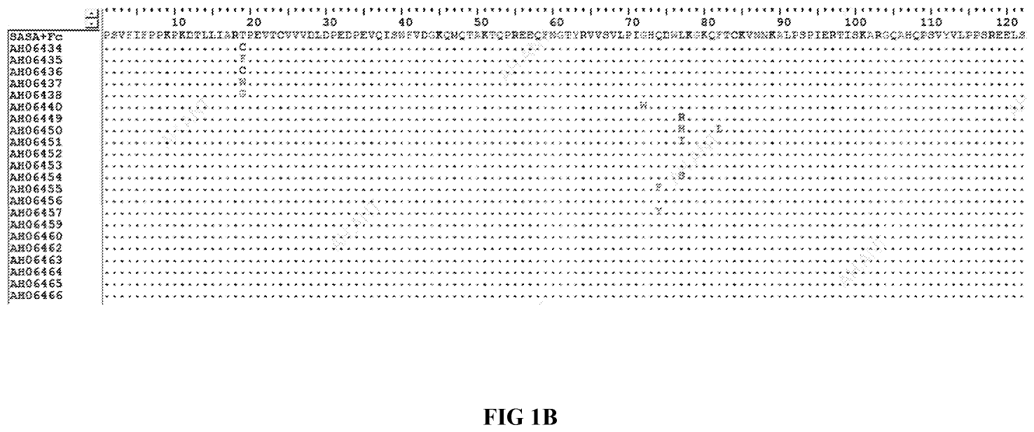

Twelve single site NNK mutation libraries of canine IgG-B Fc were prepared such that each library should have included variant IgG-B Fc polypeptides having each of the 20 possible amino acids substituted at each of the twelve sites. Each phage library was panned against canine FcRn/B2M complex at pH 6.0. After three rounds of panning, a total of 53 Fc phage clones were identified as potentially having enhanced FcRn/B2M binding and the mutations were identified by sequencing (see FIG. 1).

Single E. coli colonies expressing each of the 53 variant canine IgG-B Fc polypeptides with an SASA tag were cultured and induced to express the Fc polypeptides. Cell culture media containing the variant canine IgG-B Fc polypeptides was exposed to immobilized BSA either on a plate or a Biacore chip. The plates or chips with bound variant canine IgG-B Fc polypeptides were exposed to soluble canine FcRn/B2M complex to screen for slow off rate (koff) at pH 6. Each variant IgG-B Fc polypeptide exhibiting a slower koff with canine FcRn/B2M complex compared to wildtype IgG-B Fc polypeptide was identified. Four lead variant canine IgG-B polypeptides were identified: L(24)Y (SEQ ID NO: 14); L(24)F (SEQ ID NO: 15); L(24)M (SEQ ID NO: 16); and L(24)S (SEQ ID NO: 17).

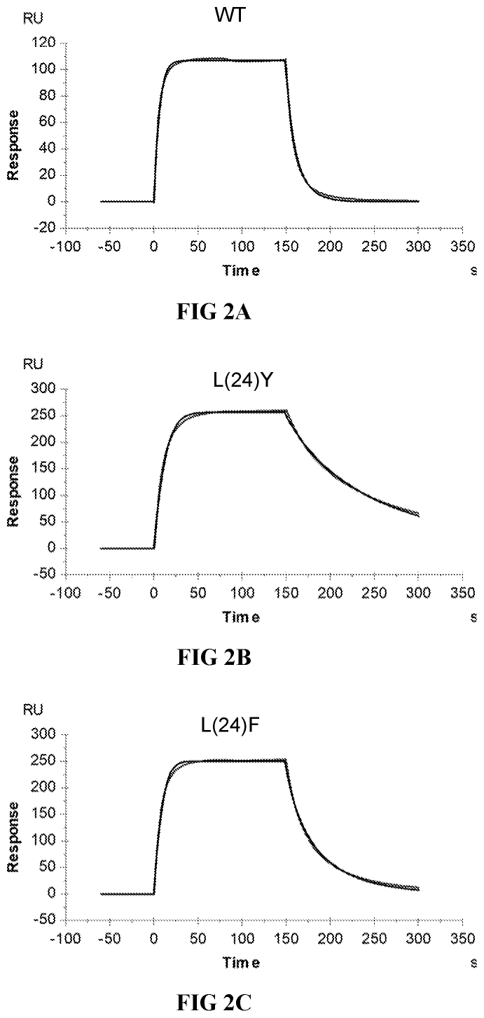

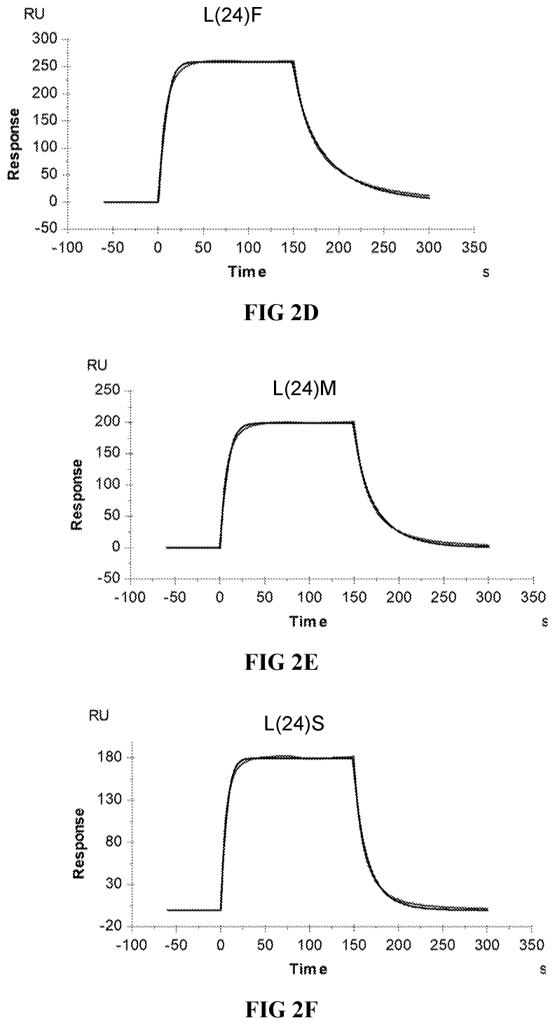

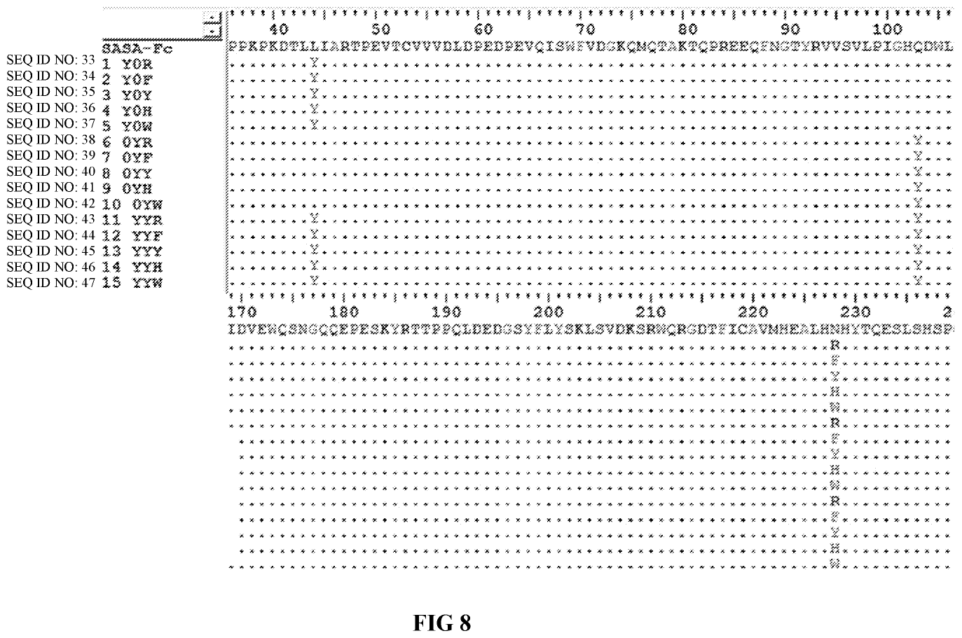

The koff of each of the lead variant canine IgG-B polypeptides was further investigated. Biotinylated canine FcRn/B2M complex was immobilized on a Biacore chip and exposed to each variant canine IgG-B polypeptide as an analyte using a Biacore T200 at pH 6.0. The results are presented in FIG. 2. The koff (1/s) for wild-type canine IgG-B Fc polypeptide was 1.22×10¹ (FIG. 2A); the koff (1/s) for variant canine IgG-B Fc polypeptide L(24)Y was 1.38×10⁻² (FIG. 2B); the koff (1/s) for variant IgG-B Fc polypeptide L(24)F was 6.31×10⁻² (FIG. 2C) and 8.47×10⁻² (FIG. 2D); the koff (1/s) for variant canine IgG-B polypeptide L(24)M was 1.26×10⁻¹ (FIG. 2E); and the koff (1/s) for variant canine IgG-B polypeptide L(24)S was 2.41×10⁻¹ (FIG. 2F).

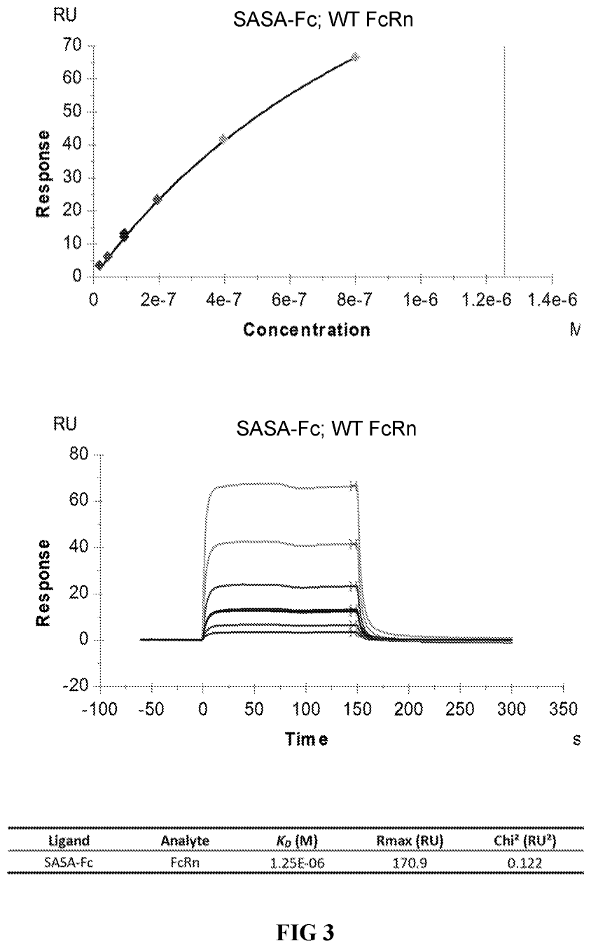

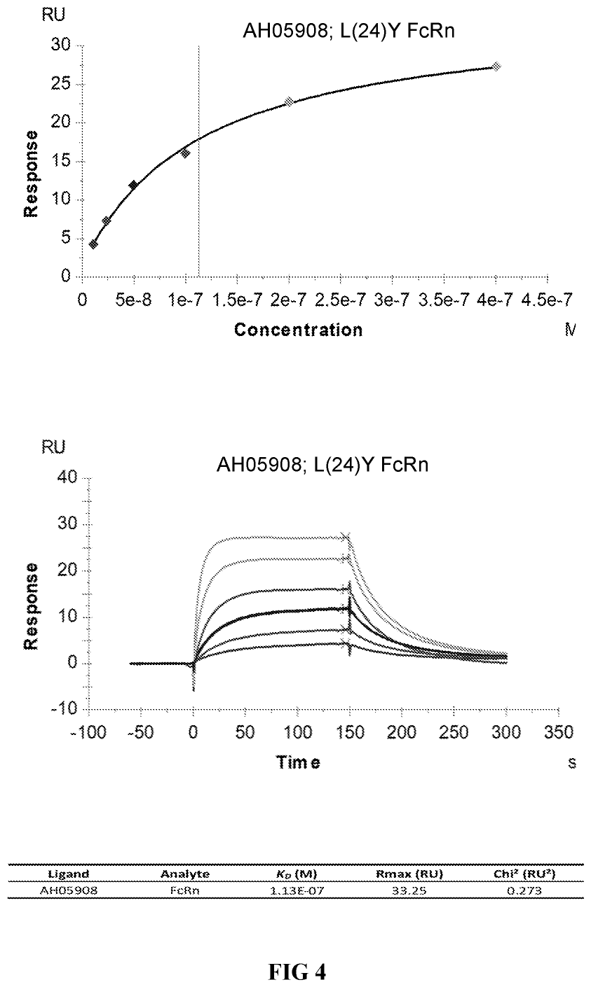

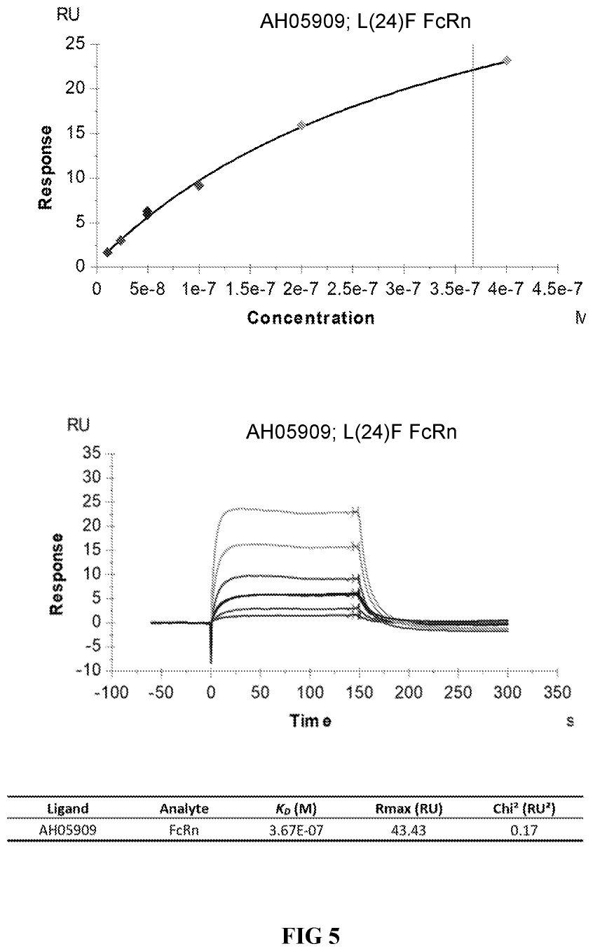

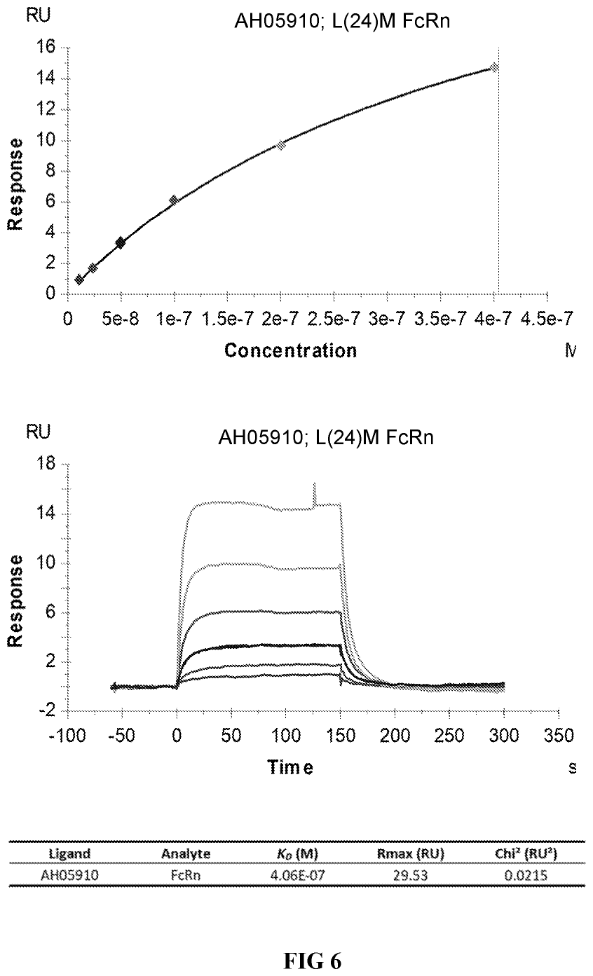

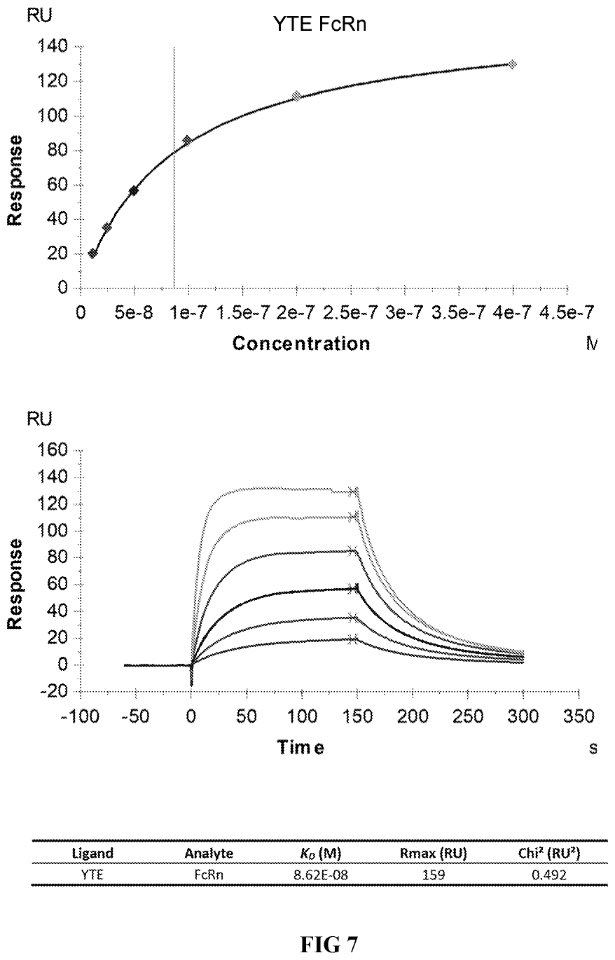

Binding analysis was performed using a Biacore T200. Briefly, the lead variant canine IgG-B Fc polypeptides with an SASA tag were each immobilized to a Series S Sensor Chip CM5. Association of each variant IgG-B Fc polypeptide with various concentrations of canine FcRn/B2M complex (12.5, 25, 50, 100, and 200 nM) was monitored at 25° C. until steady state was reached. A running buffer of 10 mM HEPES, 500 mM NaCl, 3 mM EDTA, 0.005% Tween-20, pH 6.0 was used. A buffer only blank curve was used as a control. The results are presented in FIGS. 3-7. The steady state Kd for wild-type canine IgG-B Fc polypeptide was 1.25×10⁻⁶ (FIG. 3); the steady state Kd for variant canine IgG-B Fc polypeptide L(24)Y was 1.13×10⁻⁷ (FIG. 4); the steady state Kd for variant canine IgG-B Fc polypeptide L(24)F was 3.67×10⁻⁷ (FIG. 5); and the steady state Kd for variant canine IgG-B Fc polypeptide L(24)M was 4.06×10⁻⁷ (FIG. 6); and the steady state Kd for variant canine IgG-B Fc polypeptide YTE was 8.62×10⁻⁸ (FIG. 7).

### Example 17

### Combinations of IgG Fc Mutations for Enhanced FcRn/B2M Binding at Acidic pH

ELISA screens were conducted to further identify combinations of IgG mutations having enhanced binding to FcRn/B2M. FIG. 8 and Table 17 (below) list 15 different combinations of substitutions tested at amino acid positions corresponding to position Leu24 (e.g., Leu24Tyr), position Gln83 (e.g., Gln83Tyr), and/or position Asn208 (e.g., Asn208Arg, Asn208Phe, Asn208Tyr, Asn208His, and Asn208Trp) of wild-type canine IgG-B Fc (SEQ ID NO: 2). The affinity of the 15 variant IgG Fc polypeptides to FcRn/B2M was increased compared to the affinity of either wildtype canine IgG-B Fc polypeptide (SEQ ID NO: 2) or YTE variant canine IgG-B Fc polypeptide (Leu24Tyr, Ala26Thr, Thr28Glu; SEQ ID NO: 48) to FcRn/B2M.

Briefly, each of the 15 variant canine IgG-B Fc polypeptides, wildtype canine IgG-B Fc polypeptide, and YTE variant canine IgG-B Fc polypeptide (each with an SASA tag) was subcloned into plasmid pFASEBA. Single E. coli colonies expressing the recombinant plasmids were cultured and induced to express the Fc polypeptides.

Canine FcRn with a poly-His tag (SEQ ID NO: 18) and canine B2M (SEQ ID NO: 20) heterodimer complex was transiently expressed in HEK cells and purified using Ni-NTA chromatography.

Binding analysis was performed using a Biacore 8K. Cell culture media containing the Fc polypeptides was exposed to immobilized BSA on a Biacore chip. The chips with bound Fc polypeptides were exposed to soluble canine FcRn/B2M complex (400 nM) at a flow rate of 30 μl/min to determine affinity at pH 6. A running buffer of 10 mM HEPES, 500 mM NaCl, 3 mM EDTA, 0.005% Tween-20, pH 6.0 was used. Association of each variant IgG-B Fc polypeptide with canine FcRn/B2M complex was monitored for 150 seconds at 25° C. Dissociation was monitored for 150 seconds using the running buffer. A buffer only blank curve was used as a control. The surface of the chip was regenerated between samples using a 10 mM Glycine-HCl buffer for 30 seconds at a flow rate of 30 μl/min. The affinities are presented in Table 17, below.

TABLE 17
Affinity of Variant Fc Polypeptides to FcRn/B2M

| | SEQ | | | | | |
|---|---|---|---|---|---|---|
| Variant | ID | kₒₙ | k_{off} | K_{D} | Rmax | Chi² |
| Fc | NO: | (1/Ms) | (1/s) | (M) | (RU) | (RU²) |
| 1 Y0R | 33 | 3.41E+05 | 2.95E−03 | 8.64E−09 | 26.4 | 1.07E−01 |
| 2 Y0F | 34 | 2.81E+05 | 2.21E−04 | 7.87E−10 | 50.9 | 6.42E−01 |
| 3 Y0Y | 35 | 2.86E+05 | 1.40E−04 | 4.90E−10 | 36.4 | 4.24E−01 |
| 4 Y0H | 36 | 3.34E+05 | 2.71E−03 | 8.11E−09 | 19.3 | 5.31E−02 |
| 5 Y0W | 37 | 2.50E+05 | 9.29E−04 | 3.72E−09 | 22.4 | 8.71E−02 |
| 6 0YR | 38 | 6.93E+05 | 6.55E−03 | 9.45E−09 | 64.7 | 4.89E−01 |
| 7 0YF | 39 | 3.80E+05 | 1.18E−03 | 3.10E−09 | 68.4 | 9.96E−01 |
| 8 0YY | 40 | 4.25E+05 | 8.60E−04 | 2.03E−09 | 40.9 | 2.32E−01 |
| 9 0YH | 41 | 6.73E+05 | 7.91E−03 | 1.18E−08 | 61.7 | 4.39E−01 |
| 10 0YW | 42 | 3.48E+05 | 2.13E−03 | 6.10E−09 | 29.9 | 8.52E−02 |
| 11 YYR | 43 | 3.47E+05 | 6.38E−04 | 1.84E−09 | 31.6 | 1.91E−01 |
| 12 YYF | 44 | 2.71E+05 | 1.63E−07 | 6.02E−13 | 48.5 | 7.55E−01 |
| 13 YYY | 45 | 2.62E+05 | 1.87E−07 | 7.13E−13 | 37.6 | 8.24E−01 |
| 14 YYH | 46 | 3.60E+05 | 6.12E−04 | 1.70E−09 | 42.3 | 1.26E−01 |
| 15 YYW | 47 | 2.56E+05 | 4.96E−04 | 1.93E−09 | 16.4 | 4.22E−02 |
| 16 | 2 | 8.38E+05 | 8.56E−02 | 1.02E−07 | 12.5 | 1.08E−01 |
| Wildtype | | | | | | |
| 17 YTE | 48 | 6.62E+05 | 2.05E−02 | 3.10E−08 | 16.6 | 7.59E−02 |

Each of the 15 variant IgG-B Fc polypeptides (SEQ ID NOs: 33-47) exhibited a slower koff with canine FcRn/B2M complex compared to wildtype IgG-B Fc polypeptide (SEQ ID NO: 2) and YTE variant canine IgG-B Fc polypeptide (SEQ ID NO: 48). The dissociation of YYF (Leu24Tyr, Gln83Tyr, Asn208Phe; SEQ ID NO: 44) and YYY (Leu24Tyr, Gln83Tyr, Asn208Tyr; SEQ ID NO: 45) variant IgG Fc polypeptides exceeded the detection limit of the Biacore instrument.

Corresponding substitutions (e.g., Y0R, Y0F, Y0Y, Y0H, Y0W, 0YR, 0YF, 0YY, 0YH, 0YW, YYR, YYF, YYY, YYH, and YYW) can be made to feline, equine, and other canine IgG Fc polypeptides for enhanced affinity to FcRn. For example, combinations of substitutions at amino acid positions at position 24 (e.g., substitution with Tyr), position 83 (e.g., substitution with Tyr), and/or position 208 (e.g., substitution with Arg, Phe, Tyr, His, or Trp) of wild-type canine IgG-B Fc (SEQ ID NO: 2) may be made at corresponding amino acid positions of a feline IgG1 and/or IgG2 Fc polypeptide (e.g., SEQ ID NOs: 12 and 13), an equine IgG1, IgG2, IgG3, IgG4, IgG5, IgG6, and/or IgG7 Fc polypeptide (e.g., SEQ ID NOs: 5, 6, 7, 8, 9, 10, and 11), and/or a canine IgG-A, IgG-C, and/or IgG-D Fc polypeptide (e.g., SEQ ID NOs: 1, 3, and 4).

### Example 18

### Recombinant Caninized Antibody With Variant IgG Fc Having Enhanced Affinity for FcRn

Full length antibodies having 1) a caninized variable heavy chain of mouse anti-canine IL31 clone M14 and either a wild-type canine IgG-B Fc polypeptide (SEQ ID NO: 49), a YYY variant canine IgG-B Fc polypeptide (SEQ ID NO: 50), a Y0Y variant canine IgG-B Fc polypeptide (SEQ ID NO: 51), or a YTE variant canine IgG-B Fc polypeptide (SEQ ID NO: 52) and 2) a caninized light chain of mouse anti-canine IL31 clone M14 and canine kappa light chain (SEQ ID NO: 53) were prepared. The heavy chain and light chain nucleotide sequences were synthesized chemically and inserted into an expression vector suitable for transfection into a mammalian host cell. Following transfection of heavy and light chain vector pairs into cells and culture, antibodies were purified from the culture media by single step Protein A column chromatography.

Affinity of the four antibodies to canine FcRn/B2M complex was measured using a Biacore T200. Briefly, Biotinylated FcRn/B2M complex was captured on streptavidin sensor tips. Association of FcRn/B2M complex with various concentrations of each of the four antibodies was monitored for 120 seconds at a flow rate of 30 μl/min. Concentrations of 18.75, 37.5, 75, 150, 300 600, and 1200 nM were tested for the antibody having a wild-type canine IgG-B Fc polypeptide (SEQ ID NO: 49); concentrations of 12.5, 25, 50, 100, 200, 400, and 800 nM were tested for the antibody having a YTE variant canine IgG-B Fc polypeptide (SEQ ID NO: 52); concentrations of 2.5, 5, 10, 20, 40, 80, and 160 nM were tested for the antibody having a Y0Y variant canine IgG-B Fc polypeptide (SEQ ID NO: 51); and concentrations of 1.5625, 3.125, 6.25, 12.5, 25, 50, and 100 nM were tested for the antibody having a YYY variant canine IgG-B Fc polypeptide (SEQ ID NO: 50). Higher concentrations of antibodies having wild-type and YTE Fc polypeptides were necessary to detect a signal since those Fc polypeptides had less affinity to FcRn/B2M than YYY and Y0Y Fc polypeptides (see Example 17).

Dissociation was monitored for 120 seconds for antibodies having wild-type, a YTE variant, or a Y0Y variant canine IgG-B Fc polypeptide or for 300 seconds for the antibody having a YYY variant canine IgG-B Fc polypeptide. A running buffer of 10 mM HEPES, 500 mM NaCl, 3 mM EDTA, 0.005% Tween-20, pH 6.0 was used. A buffer only blank curve was used as a control. The surface of the chip was regenerated between samples using a 10 mM Glycine-HCl buffer for 40 seconds at a flow rate of 30 μl/min.

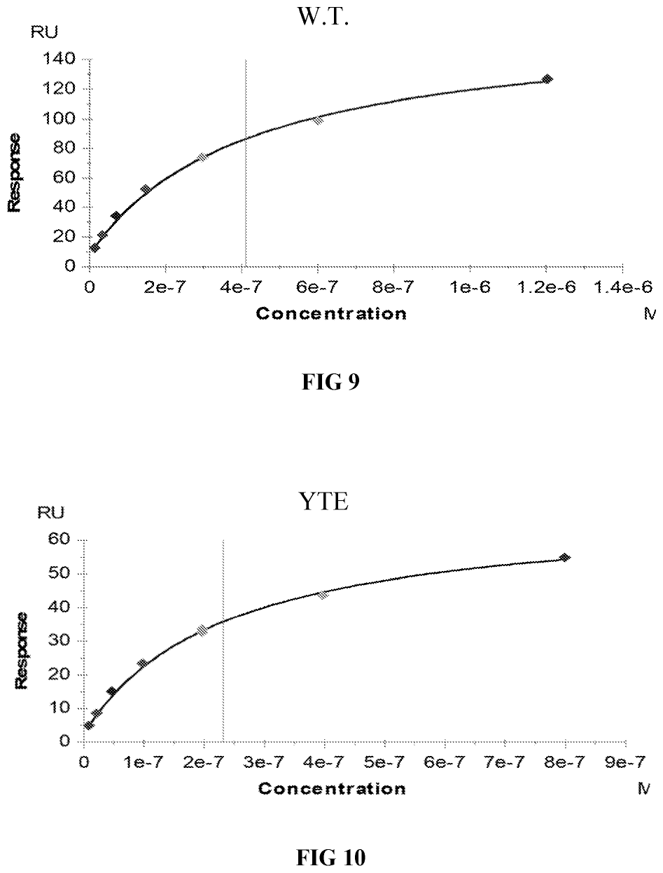

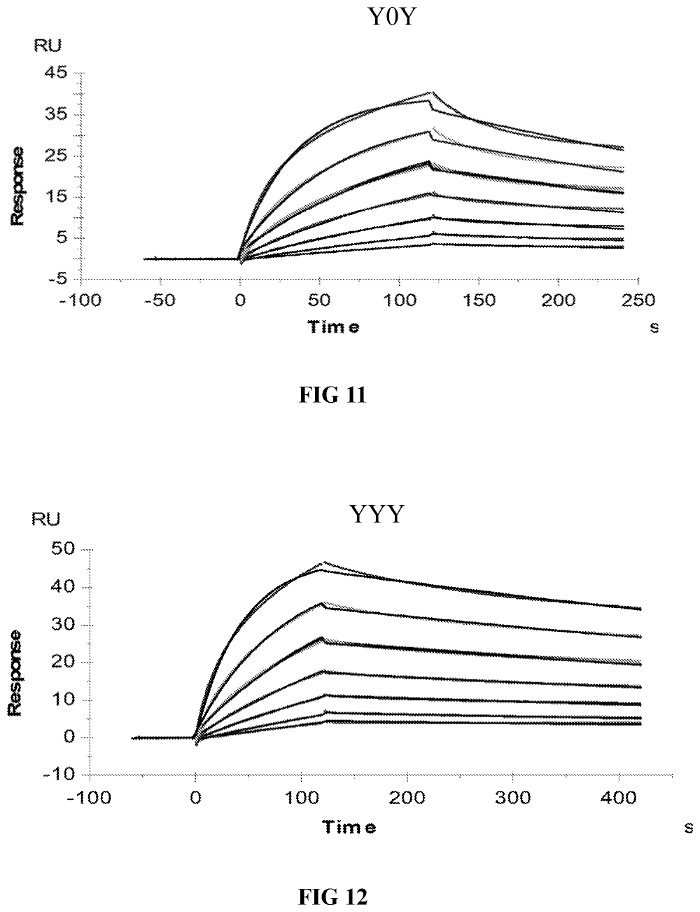

The affinities are presented in Table 18 (below). Due to the low affinity of wild-type canine IgG-B Fc and YTE variant canine IgG-B Fc for FcRn/B2M, rate constants were not well observed for those antibodies. Thus, steady state binding analyses were performed in a manner similar to that described above to obtain binding affinities. Affinity plots for antibodies having wild-type and YTE variant canine IgG-B Fc polypeptides are presented in FIG. 9 and FIG. 10, respectively. Sensor-grams for antibodies having Y0Y and YYY variant IgG-B Fc polypeptides are presented in FIG. 11 and FIG. 12, respectively.

TABLE 18
Affinity of Antibodies having Variant
Fc Polypeptides to FcRn/B2M

| | SEQ | | | | | |
|---|---|---|---|---|---|---|
| IgG-B | ID | kₒₙ | k_{off} | K_{D} | Rmax | Chi² |
| Fc | NO: | (1/Ms) | (1/s) | (M) | (RU) | (RU²) |
| Y0Y | 51 | 1.68E+05 | 2.62E−03 | 1.56E−08 | 40.89 | 0.231 |
| YYY | 50 | 2.27E+05 | 8.69E−04 | 3.84E−09 | 48.91 | 0.137 |
| Wildtype | 49 | N/A | N/A | 4.10E−07 | 157.7 | 5.12 |
| YTE | 52 | N/A | N/A | 2.31E−07 | 66.93 | 0.628 |

### Example 19

### YTE Mutation in Feline IgG Enhances Feline FcRn Interaction

The affinity of variant feline Fc polypeptides for FcRn was evaluated in the context of chimeric D2E7 anti-TNFα antibodies. Anti-TNFα variable heavy (VH) and variable light (VL) chain sequences derived from D2E7 were fused to feline constant sequences. Specifically, D2E7 VL chain was fused to feline kappa light chain to produce SEQ ID NO: 117. In addition, D2E7 VH chain was fused to wild-type feline IgG1b Fc polypeptide (comprising SEQ ID NO: 13) or YTE variant feline IgG1b Fc polypeptide (comprising SEQ ID NO: 82) to produce SEQ ID NOs: 119 and 121, respectively.

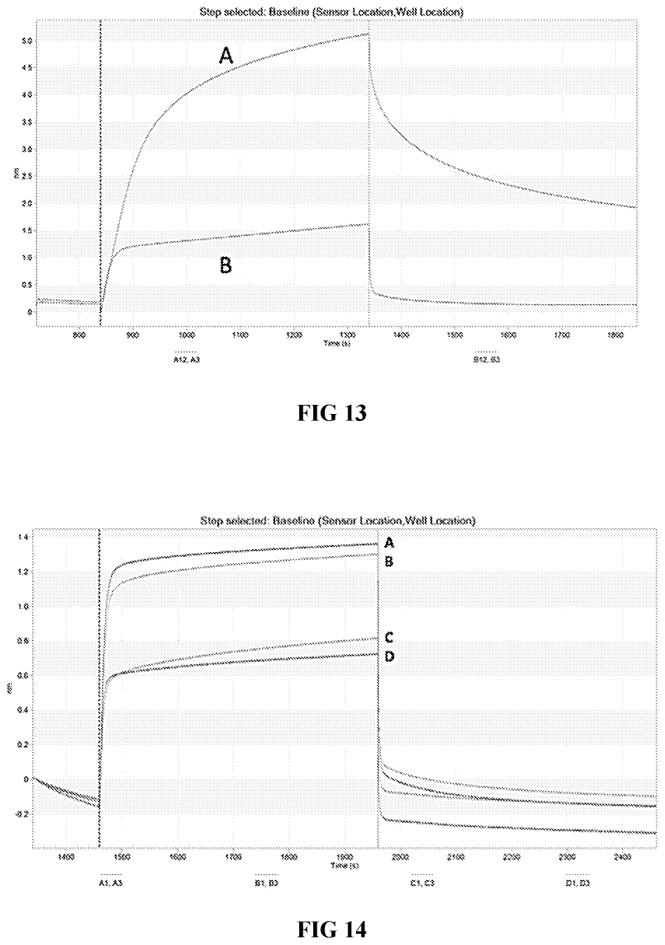

The binding analysis was performed using a biosensor OctetRed as follows. Briefly, biotinylated feline FcRn was captured on streptavidin sensor tips. The association of antibody at 30 μg/mL was measured at pH 6.0 and dissociation was performed at pH 7.2. The YTE mutations in feline IgG Fc (FIG. 13, A) enhanced association to feline FcRN at low pH compared to wild-type feline IgG Fc (FIG. 13, B).

### Example 20

### Phe Mutation in Canine IgG Enhances Canine FcRn Interaction

The affinity of variant canine Fc polypeptides for FcRn was evaluated in the context of chimeric D2E7 anti-TNFα antibodies. Anti-TNFα variable heavy (VH) and variable light (VL) chain sequences derived from D2E7 were fused to canine constant sequences. Specifically, D2E7 VL chain was fused to canine kappa light chain to produce SEQ ID NO: 93. In addition, D2E7 VH chain was fused to variant canine IgG-A Fc polypeptides comprising SEQ ID NO: 85 (F00; Protein A+; C1q−; CD16−) or SEQ ID NO: 86 (Protein A+; C1q+; CD16+) and to variant canine IgG-D Fc polypeptides comprising SEQ ID NO: 89 (F00; Protein A+; C1q−; CD16−), or SEQ ID NO: 90 (Protein A+; C1q+; CD16+) to produce SEQ ID NOs: 110, 107, 112, and 115, respectively.

The binding analysis was performed using a biosensor OctetRed as follows. Briefly, biotinylated TNFα was captured on streptavidin sensor tips. The association of antibody at 20 μg/mL was bound to TNFα. The complex was then used to bind to canine FcRn (50 μg/mL) at pH 6.0. Dissociation was performed at pH 7.2.

The Phe mutation enhanced canine FcRn binding at low pH (pH6.0, 20 mM NaCitrate, 140 mM NaCl), as illustrated by the binding profiles of chimeric variant canine IgG-A “F00” antibody (SEQ ID NO: 93 and SEQ ID NO: 110) (FIG. 14, A) and IgG-D “F00” antibody (SEQ ID NO: 93 and SEQ ID NO: 115) (FIG. 14, B) compared to chimeric variant canine IgG-A without the Phe mutation (SEQ ID NO: 93 and SEQ ID NO: 107) (FIG. 14, C) and IgG-D without the Phe mutation (SEQ ID NO: 93 and SEQ ID NO: 112) (FIG. 14, D). The chimeric variant canine IgG-A and IgG-D antibodies with the Phe mutation (FIG. 14, A and B) exhibited enhanced association with canine FcRn at low pH (pH 6.0) and fast dissociation at neutral pH (PBS pH7.2). A similar enhanced binding profile was also observed with chimeric variant canine IgG-B “F00” antibody (SEQ ID NO: 93 and SEQ ID NO: 105).

### Example 21

### Pharmacokinetics of Phe Mutation in Canine IgG

Pharmacokinetics analysis was performed using Sprague Dawley rats. The rats were subcutaneously administered with 2 mg/kg of chimeric variant canine IgG-A “F00” antibody (SEQ ID NO: 93 and SEQ ID NO: 110) and chimeric variant canine IgG-A without the Phe mutation (SEQ ID NO: 93 and SEQ ID NO: 107) (two rats per group). Serum samples were collected from the rats at pre-injection and at 0.5, 1, 6, 24, 48, 72, 168, 216, and 336 hours post injection. The canine chimeric antibody concentrations in the serum samples were determined by ELISA, as follows.

Anti-adalimumab antibody (Bio-Rad, catalog no. HCA202) 1 μg/mL in PBS was coated on a 96-well Maxisorp plate with 100 μl in each well. The plate was incubated overnight at 4° C. and washed five times with PBST (PBS containing 0.05% Tween-20). Each well was blocked with 200 μl 5% BSA in PBST and the plate incubated for 1 hour at room temperature. The plate was washed five times with PBST. Anti-TNFα antibody at a concentration of 1,000 ng/mL was prepared in 5% BSA-PBST along with serial dilutions to 0.1 ng/mL. The anti-TNFα serial dilutions (1,000 ng/mL to 0.1 ng/mL) were added to the plate in duplicate and along with a blank well containing no anti-TNFα were used to generate a standard curve. The serum samples were prepared by 10-fold, 20-fold, and 40-fold dilutions in 5% BSA-PBST and added to the plate. The plate was incubated at room temperature for 1 hour and washed 5 times with PBST. 100 μl HRP-conjugated antibody (Bio-Rad, catalog no. HCA204P) was added to each well at 0.25 μg/mL in 5% BSA-PBST. The plate was incubated for 1 hour at room temperature and washed 5 times with PBST. 100 μl QuantaBlu (Thermo Scientific, catalog no. 15169) was added to each well. The fluorescence was measured after 10-15 minutes incubation at 325 nm/420 nm (emission/excitation). The titer of anti-TNFα in the serum samples was calculated against the standard curve.

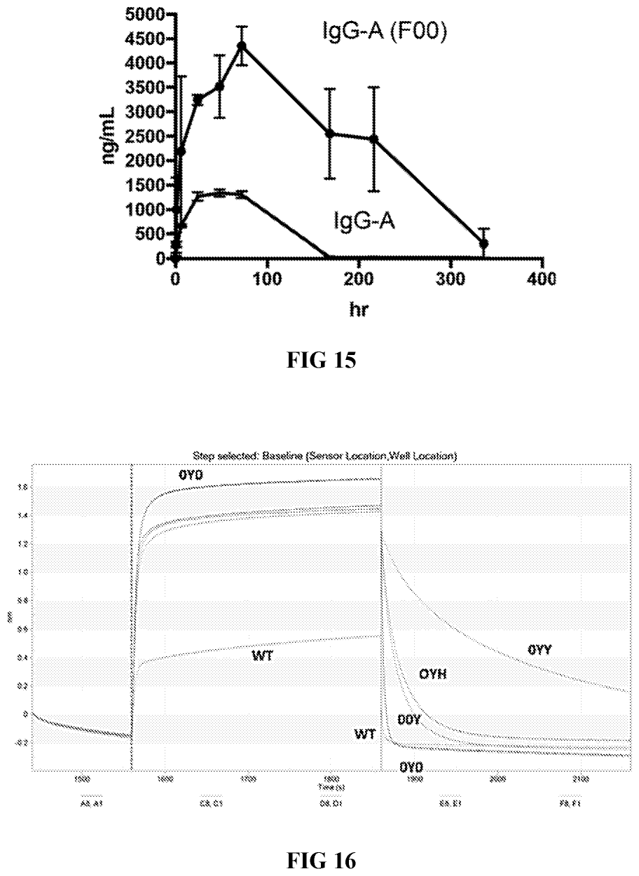

The AUC₀₋₃₃₆ₕ for IgG-A was 150970, while IgG-A “F00” was 848924 ng/mL*hr (FIG. 15). The terminal half-life was estimated to be 33 hours and 152 hours, respectively. Thus, the single Phe mutation significantly improved the pharmacokinetic profile of the anti-TNFα antibody in rat.

### Example 22

### Phe Mutation in Canine, Feline, and Equine IgG Fcs

The interaction between the Phe mutation in canine IgG-A, IgG-B, IgG-C, and IgG-D Fc and FcRn was modeled using three-dimensional protein structure analysis. The aromatic side chain of Phe appears to have a hydrophobic interaction with canine FcRn at the Pro hydrophobic ring (π-CH) of the “WPE” motif. In addition, the Phe hydrophobic side chain may be in direct contact with the Glu side chain next to the Pro of the same “WPE” motif. This interaction may have energy penalty if the Glu side chain is deprotonated to be negative charged, such as at a neutral pH. Thus, some level of protonation of the Glu residue may be required to minimize the aromatics to Glu-H interaction. That may explain why the interaction between variant IgGs having the Phe mutation and FcRn is reduced at neutral pH. Based on protein structure analysis, the interaction appears to be conserved among canine IgG-A, IgG-B, IgG-C, and IgG-D Fc.

Furthermore, the interactions between a Phe mutation in feline IgG1a and IgG2 Fc were modeled when complexed with feline FcRn. The same interactions observed with the canine IgG Fcs appeared to be conserved with the feline IgG Fcs.

The interactions between a Phe mutation in equine IgG1, IgG2, IgG3, IgG4, IgG5, IgG6, and IgG7 Fc in complex with equine FcRn were also modeled. The same interactions appeared to be maintained with the equine IgG Fcs.

### Example 23

### Other Exemplary Variant Canine IgG Fcs Enhance Canine FcRn Interaction

The affinity of additional variant canine Fc polypeptides for FcRn was evaluated in the context of chimeric D2E7 anti-TNFα antibodies. Anti-TNFα variable heavy (VH) and variable light (VL) chain sequences derived from D2E7 were fused to canine constant sequences. Specifically, D2E7 VL chain was fused to canine kappa light chain to produce SEQ ID NO: 93. In addition, D2E7 VH chain was fused to wild-type IgG-B Fc polypeptide (comprising SEQ ID NO: 2), variant canine IgG-B Fc polypeptide 0Y0 (comprising SEQ ID NO: 27), variant canine IgG-B Fc polypeptide 0YH (comprising SEQ ID NO: 41), variant canine IgG-B Fc polypeptide 0YY (comprising SEQ ID NO: 40), and variant canine IgG-B Fc polypeptide 00Y (comprising SEQ ID NO: 30) to produce SEQ ID NOs: 95, 97, 99, 101, and 103, respectively.

The binding analysis was performed using a biosensor OctetRed as follows. Briefly, biotinylated TNFα was captured on streptavidin sensor tips. The association of antibody at 20 μg/mL was bound to TNFα. The complex was then used to bind to canine FcRn (50 μg/mL) at pH 6.0. Dissociation was performed at pH 7.2.

Each of the chimeric variant canine IgG-B antibodies exhibited enhanced binding to canine FcRn at pH 6.0 compared to the chimeric wild-type canine IgG-B antibody and each had an appreciable rate of dissociation at neutral pH (FIG. 16).

1. A polypeptide comprising a variant IgG Fc polypeptide comprising at least one amino acid substitution relative to a wild-type IgG Fc polypeptide derived from a companion animal species, wherein the variant Fc polypeptide is capable of binding to neonatal Fc receptor (FcRn) with an increased affinity relative to the wild-type Fc polypeptide.

2. The polypeptide of claim 1, wherein the variant Fc polypeptide binds to FcRn with an affinity greater than the wild-type IgG Fc polypeptide, as measured by biolayer interferometry, surface plasmon resonance, or any protein-protein interaction tool at a pH in the range of from about 5.0 to about 6.5, such as at a pH of about 5.0, a pH of about 5.2, a pH of about 5.5, a pH of about 6.0, a pH of about 6.2, or a pH of about 6.5.

3. The polypeptide of claim 1 or claim 2, wherein the variant IgG Fc polypeptide binds to FcRn with a dissociation constant (Kd) of less than 5×10⁻⁶M, less than 1×10⁻⁶M, less than 5×10⁻⁷ M, less than 1×10⁻⁶ M, less than 5×10⁻⁸ M, less than 1×10⁻⁸M, less than 5×10⁻⁹M, less than 1×10⁻⁹M, less than 5×10⁻¹⁰ M, less than 1×10⁻¹⁰ M, less than 5×10⁻¹¹ M, less than 1×10⁻¹¹ M, less than 5×10⁻¹² M, or less than 1×10⁻¹² M, as measured by biolayer interferometry, surface plasmon resonance, or any protein-protein interaction tool at a pH in the range of from about 5.0 to about 6.5, such as at a pH of about 5.0, a pH of about 5.5, a pH of about 6.0, or a pH of about 6.5.

4. The polypeptide of any one of the preceding claims, wherein the polypeptide has increased serum half-life relative to a polypeptide comprising a wild-type Fc polypeptide.

5. The polypeptide of any one of the preceding claims, wherein the companion animal species is canine, feline, or equine.

6. The polypeptide of any one of the preceding claims, wherein the wild-type IgG Fc polypeptide is
a) a canine IgG-A Fc, IgG-B Fc, IgG-C Fc, or IgG-D Fc;
b) an equine IgG1 Fc, IgG2 Fc, IgG3 Fc, IgG4 Fc, IgG5 Fc, IgG6 Fc, or IgG7 Fc; or
c) a feline IgG1 Fc, or IgG2 Fc.

7. The polypeptide of any one of the preceding claims, wherein the variant IgG Fc polypeptide is at least 90% identical, at least 95% identical, at least 97% identical, or at least 99% identical to the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, or SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, or SEQ ID NO: 91.

8. The polypeptide of any one of the preceding claims, wherein the variant IgG Fc polypeptide comprises:
a) at least one amino acid substitution at at least one position corresponding to position 9, 18, 22, 23, 24, 25, 26, 27, 28, 30, 31, 60, 79, 80, 83, 132, 149, 162, 183, 203, 205, and/or 209 of SEQ ID NO: 1;
b) at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 2;
c) at least one amino acid substitution at at least one position corresponding to position 9, 18, 22, 23, 24, 26, 27, 28, 30, 31, 60, 79, 80, 81, 83, 132, 148, 162, 182, 202, 204, and/or 208 of SEQ ID NO: 3;
d) at least one amino acid substitution at at least one position corresponding to position 9, 18, 22, 23, 24, 25, 26, 27, 28, 30, 60, 79, 80, 83, 132, 149, 163, 183, 203, 205, and/or 209 of SEQ ID NO: 4;
e) at least one amino acid substitution at at least one position corresponding to position 9, 18, 22, 23, 24, 25, 26, 27, 28, 30, 31, 79, 80, 81, 83, 132, 148, 162, 182, 202, 204, 208, and/or 213 of SEQ ID NO: 5;
f) at least one amino acid substitution at at least one position corresponding to position 9, 18, 22, 23, 24, 25, 26, 27, 28, 79, 80, 81, 83, 132, 148, 162, 182, 202, 204, 208, and/or 213 of SEQ ID NO: 6;
g) at least one amino acid substitution at at least one position corresponding to position 9, 18, 22, 23, 24, 25, 27, 28, 30, 31, 60, 79, 80, 81, 83, 132, 148, 162, 182, 202, 204, 208, and/or 213 of SEQ ID NO: 7;
h) at least one amino acid substitution at at least one position corresponding to position 9, 18, 22, 23, 24, 25, 27, 28, 30, 31, 79, 80, 83, 132, 148, 162, 182, 202, 204, 208, and/or 213 of SEQ ID NO: 8;
i) at least one amino acid substitution at at least one position corresponding to position 9, 18, 22, 23, 24, 25, 27, 28, 30, 31, 79, 80, 81, 83, 132, 148, 162, 182, 202, 204, 208 and/or 213 of SEQ ID NO: 9;
j) at least one amino acid substitution at at least one position corresponding to position 18, 22, 23, 24, 25, 27, 28, 30, 31, 79, 80, 81, 83, 132, 148, 162, 182, 202, 204, 208, and/or 213 of SEQ ID NO: 10;
k) at least one amino acid substitution at at least one position corresponding to position 18, 22, 23, 24, 25, 27, 28, 30, 31, 79, 80, 81, 83, 132, 148, 162, 182, 202, 204, 208, and/or 213 of SEQ ID NO: 11;
l) at least one amino acid substitution at at least one position corresponding to position 9, 18, 22, 23, 24, 25, 26, 27, 28, 30, 31, 60, 79, 80, 81, 83, 148, 162, 182, 202, 204, 208, and/or 213 of SEQ ID NO: 12;
m) at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 13; or n) at least one amino acid substitution at at least one position corresponding to position 9, 18, 22, 23, 24, 25, 26, 27, 28, 30, 31, 60, 79, 80, 81, 83, 132, 148, 162, 182, 202, 204, 208, and/or 213 of SEQ ID NO: 54.

9. The polypeptide of any one of the preceding claims, wherein the variant IgG Fc polypeptide comprises:
a) at least one amino acid substitution at at least one position selected from 9, 18, 22, 23, 24, 25, 26, 27, 28, 30, 31, 60, 79, 80, 83, 132, 149, 162, 183, 203, 205, and/or 209 of SEQ ID NO: 1;
b) at least one amino acid substitution at at least one position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 2;
c) at least one amino acid substitution at at least one position selected from position 9, 18, 22, 23, 24, 26, 27, 28, 30, 31, 60, 79, 80, 81, 83, 132, 148, 162, 182, 202, 204, and/or 208 of SEQ ID NO: 3;
d) at least one amino acid substitution at at least one position selected from position 9, 18, 22, 23, 24, 25, 26, 27, 28, 30, 60, 79, 80, 83, 132, 149, 163, 183, 203, 205, and/or 209 of SEQ ID NO: 4;
e) at least one amino acid substitution at at least one position selected from position 9, 18, 22, 23, 24, 25, 26, 27, 28, 30, 31, 79, 80, 81, 83, 132, 148, 162, 182, 202, 204, 208, and/or 213 of SEQ ID NO: 5;
f) at least one amino acid substitution at at least one position selected from position 9, 18, 22, 23, 24, 25, 26, 27, 28, 79, 80, 81, 83, 132, 148, 162, 182, 202, 204, 208, and/or 213 of SEQ ID NO: 6;
g) at least one amino acid substitution at at least one position selected from position 9, 18, 22, 23, 24, 25, 27, 28, 30, 31, 60, 79, 80, 81, 83, 132, 148, 162, 182, 202, 204, 208, and/or 213 of SEQ ID NO: 7;
h) at least one amino acid substitution at at least one position selected from position 9, 18, 22, 23, 24, 25, 27, 28, 30, 31, 79, 80, 83, 132, 148, 162, 182, 202, 204, 208, and/or 213 of SEQ ID NO: 8;
i) at least one amino acid substitution at at least one position selected from position 9, 18, 22, 23, 24, 25, 27, 28, 30, 31, 79, 80, 81, 83, 132, 148, 162, 182, 202, 204, 208 and/or 213 of SEQ ID NO: 9;
j) at least one amino acid substitution at at least one position selected from position 18, 22, 23, 24, 25, 27, 28, 30, 31, 79, 80, 81, 83, 132, 148, 162, 182, 202, 204, 208, and/or 213 of SEQ ID NO: 10;
k) at least one amino acid substitution at at least one position selected from position 18, 22, 23, 24, 25, 27, 28, 30, 31, 79, 80, 81, 83, 132, 148, 162, 182, 202, 204, 208, and/or 213 of SEQ ID NO: 11;
l) at least one amino acid substitution at at least one position selected from position 9, 18, 22, 23, 24, 25, 26, 27, 28, 30, 31, 60, 79, 80, 81, 83, 148, 162, 182, 202, 204, 208, and/or 213 of SEQ ID NO: 12; or
m) at least one amino acid substitution at at least one position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 13; or
n) at least one amino acid substitution at at least on position selected from position 9, 18, 22, 23, 24, 25, 26, 27, 28, 30, 31, 60, 79, 80, 81, 83, 132, 148, 162, 182, 202, 204, 208, and/or 213 of SEQ ID NO: 54.

10. The polypeptide of any one of the preceding claims, wherein the variant IgG Fc polypeptide comprises:
a) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 149; a leucine, a phenylalanine, or a tyrosine at a position corresponding to position 162; an isoleucine at a position corresponding to position 183; a leucine or a tryptophan at a position corresponding to position 203; a proline at a position corresponding to position 205; and/or a serine, an alanine, an arginine, a phenylalanine, a tyrosine, a histidine, or a tryptophan at a position corresponding to position 209 of SEQ ID NO: 1;
b) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, tyrosine, methionine, arginine, or valine at a position corresponding to position 24; a threonine at a position corresponding to position 25; a tryptophan, a tyrosine, a histidine, a leucine, a phenylalanine, a valine, a methionine, a threonine, or a serine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 2;
c) a tyrosine at a position corresponding to position 9; a proline at a position corresponding to position 18; a histidine or a proline at a position corresponding to position 22; a valine, an arginine, or a lysine at a position corresponding to position 23; a phenylalanine, a tyrosine, a methionine, or an arginine at a position corresponding to position 24; a tryptophan, a tyrosine, a histidine, a leucine, a phenylalanine, a valine, a methionine, a threonine, or a serine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; and/or a serine, an alanine, an arginine, a phenylalanine, a tyrosine, a histidine, or a tryptophan at a position corresponding to position 208 of SEQ ID NO: 3;
d) a tyrosine at a position corresponding to position 9; a proline at a position corresponding to position 18; a histidine or a proline at a position corresponding to position 22; a valine, an arginine, or a lysine at a position corresponding to position 23; a phenylalanine, a tyrosine, a methionine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an alanine at a position corresponding to position 60; a glutamine, an arginine, an alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 149; a leucine, a phenylalanine, or a tyrosine at a position corresponding to position 163; an isoleucine at a position corresponding to position 183; a leucine or a tryptophan at a position corresponding to position 203; a proline at a position corresponding to position 205; and/or a serine, an alanine, an arginine, a phenylalanine, a tyrosine, a histidine, or a tryptophan at a position corresponding to position 209 of SEQ ID NO: 4;
e) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 5;
f) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, a methionine, or a threonine at a position corresponding to position 26; a leucine, a methionine, a proline, an isoleucine, an asparagine, or a valine at a position corresponding to position 27; a methionine, a glutamic acid, an asparagine, a histidine, an isoleucine, or a valine at a position corresponding to position 28; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83, an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 6;
g) a tyrosine at a position corresponding to position 9; a proline at a position corresponding to position 18; a histidine, an isoleucine, and/or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 7;
h) a tyrosine at a position corresponding to position 9; a proline at a position corresponding to position 18; a histidine, an isoleucine, or a proline at a position corresponding to position 22; a valine, an arginine, a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, alanine, a glutamic acid, or a methionine at a position corresponding to position 79; a histidine or a phenylalanine at a position corresponding to position 80; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 8;
i) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 9;
j) a proline at a position corresponding to position 18; a histidine, an isoleucine, or a proline at a position corresponding to position 22; a valine, an arginine, or a lysine at a position corresponding to position 23; a phenylalanine, a tyrosine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, or a tryptophan at a position corresponding to position 208; and/or an isoleucine or a valine at a position corresponding to position 213 of SEQ ID NO: 10;
k) a proline at a position corresponding to position 18; a histidine, an isoleucine, or a proline at a position corresponding to position 22; a valine, an arginine, or a lysine at a position corresponding to position 23; a phenylalanine, a tyrosine, an arginine, or a valine at a position corresponding to position 24; a threonine at a position corresponding to position 25; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; a glutamine, an arginine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; a serine, an alanine, an arginine, a phenylalanine, a tyrosine, a histidine, or a tryptophan at a position corresponding to position 208; and/or an isoleucine or a valine at a position corresponding to position 213 of SEQ ID NO: 11;
l) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; a valine at a position corresponding to position 148; a leucine, a phenylalanine, or a tyrosine at a position corresponding to position 162; an isoleucine at a position corresponding to position 182; a leucine or a tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 12;
m) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 13; or
n) a tyrosine at a position corresponding to position 9; a proline at a position corresponding to position 18; a histidine, an isoleucine, or a proline at a position corresponding to position 22; a valine, an arginine, or a lysine at a position corresponding to position 23; a phenylalanine, a tyrosine, a methionine, an arginine, or a valine at a position corresponding to position 24; a threonine at a position corresponding to position 25; a tryptophan, a tyrosine, a histidine, a leucine, a phenylalanine, a valine, or a methionine at a position corresponding to position 26; a leucine, a methionine, a proline, an isoleucine, an asparagine, or a tryptophan at a position corresponding to position 27; a methionine, a glutamic acid, an asparagine, a histidine, an isoleucine, or a valine at a position corresponding to position 28; a methionine or a valine at a position corresponding to position 30; an isoleucine at a position corresponding to position 31; an alanine at a position corresponding to position 60; a glutamine, an arginine, an alanine, a glutamic acid, or a methionine at a position corresponding to position 79; a histidine or a phenylalanine at a position corresponding to position 80; a glutamic acid at a position corresponding to position 81; an alanine, a tyrosine, or a phenylalanine at a position corresponding to position 83; an isoleucine at a position corresponding to position 132; a valine at a position corresponding to position 148; a leucine, a phenylalanine, or a tyrosine at a position corresponding to position 162; an isoleucine at a position corresponding to position 182; a leucine or tryptophan at a position corresponding to position 202; a proline at a position corresponding to position 204; an alanine, an arginine, a phenylalanine, a tyrosine, a histidine, or a tryptophan at a position corresponding to position 208; and/or an isoleucine or a valine at a position corresponding to position 213 of SEQ ID NO: 54.

11. The polypeptide of any one of the preceding claims, wherein the variant IgG Fc polypeptide comprises:
a) a tyrosine at position 9; a proline at position 18; a histidine, an isoleucine, or a proline at position 22; a valine, an arginine, or a lysine at position 23; a phenylalanine, a tyrosine, a methionine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 149; a leucine, a phenylalanine, or a tyrosine at position 162; an isoleucine at position 183; a leucine or a tryptophan at position 203; a proline at position 205; and/or a serine, an alanine, an arginine, a phenylalanine, a tyrosine, a histidine, or a tryptophan at position 209 of SEQ ID NO: 1;
b) a tyrosine at position 9; a proline at position 18; a histidine, an isoleucine, or a proline at position 22; a valine, an arginine, or a lysine at position 23; a phenylalanine, tyrosine, methionine, arginine, or valine at position 24; a threonine at position 25; a tryptophan, a tyrosine, a histidine, a leucine, a phenylalanine, a valine, a methionine, a threonine, or a serine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 2;
c) a tyrosine at position 9; a proline at position 18; a histidine or a proline at position 22; a valine, an arginine, or a lysine at position 23; a phenylalanine, a tyrosine, a methionine, or an arginine at position 24; a tryptophan, a tyrosine, a histidine, a leucine, a phenylalanine, a valine, a methionine, a threonine, or a serine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; and/or a serine, an alanine, an arginine, a phenylalanine, a tyrosine, a histidine, or a tryptophan at position 208 of SEQ ID NO: 3;
d) a tyrosine at position 9; a proline at position 18; a histidine or a proline at position 22; a valine, an arginine, or a lysine at position 23; a phenylalanine, a tyrosine, a methionine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an alanine at position 60; a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 149; a leucine, a phenylalanine, or a tyrosine at position 163; an isoleucine at position 183; a leucine or a tryptophan at position 203; a proline at position 205; and/or a serine, an alanine, an arginine, a phenylalanine, a tyrosine, a histidine, or a tryptophan at position 209 of SEQ ID NO: 4;
e) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, a glutamic acid, or a methionine at position 79, a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 5;
f) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, a methionine, or a threonine at position 26; a leucine, a methionine, a proline, an isoleucine, an asparagine, or a valine at position 27; a methionine, a glutamic acid, an asparagine, a histidine, an isoleucine, or a valine at position 28; a glutamine, an arginine, an alanine, a glutamic acid, or a methionine at position 79; a histidine or a phenylalanine at position 80; a glutamic acid at position 81; an alanine, a tyrosine, or a phenylalanine at position 83, an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 6;
g) a tyrosine at position 9; a proline at position 18; a histidine, an isoleucine, and/or a proline at position 22; a valine, an arginine, or a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 7;
h) a tyrosine at position 9; a proline at position 18; a histidine, an isoleucine, or a proline at position 22; a valine, an arginine, a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glycine, an arginine, a glutamine, an arginine, an alanine, a glutamic acid, or a methionine at position 79; a histidine or a phenylalanine at position 80; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 8;
i) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, a histidine, or a tryptophan at position 208; and/or an isoleucine or a valine at position 213 of SEQ ID NO: 9;
j) a proline at position 18; a histidine, an isoleucine, or a proline at position 22; a valine, an arginine, or a lysine at position 23; a phenylalanine, a tyrosine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, an arginine, a phenylalanine, a tyrosine, or a tryptophan at position 208; and/or an isoleucine or a valine at position 213 of SEQ ID NO: 10;
k) a proline at position 18; a histidine, an isoleucine, or a proline at position 22; a valine, an arginine, or a lysine at position 23; a phenylalanine, a tyrosine, an arginine, or a valine at position 24; a threonine at position 25; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; a glutamine, an arginine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or a tryptophan at position 202; a proline at position 204; a serine, an alanine, a phenylalanine, a tyrosine, a histidine, or a tryptophan at position 208; and/or an isoleucine or a valine at position 213 of SEQ ID NO: 11;
l) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; a valine at position 148; a leucine, a phenylalanine, or a tyrosine at position 162; an isoleucine at position 182; a leucine or a tryptophan at position 202; a proline at position 204; an alanine, an arginine, a phenylalanine, a tyrosine, a histidine, or a tryptophan at position 208; and/or an isoleucine or a valine at position 213 of SEQ ID NO: 12;
m) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or tryptophan at position 202; a proline at position 204; an alanine, an arginine, a phenylalanine, a tyrosine, a histidine, or a tryptophan at position 208; and/or an isoleucine or a valine at position 213 of SEQ ID NO: 13; or
n) a tyrosine at position 9; a proline at position 18; a histidine, an isoleucine, or a proline at position 22; a valine, an arginine, or a lysine at position 23; a phenylalanine, a tyrosine, a methionine, an arginine, or a valine at position 24; a threonine at position 25; a tryptophan, a tyrosine, a histidine, a leucine, a phenylalanine, a valine, or a methionine at position 26; a leucine, a methionine, a proline, an isoleucine, an asparagine, or a tryptophan at position 27; a methionine, a glutamic acid, an asparagine, a histidine, an isoleucine, or a valine at position 28; a methionine or a valine at position 30; an isoleucine at position 31; an alanine at position 60; a glutamine, an arginine, an alanine, a glutamic acid, or a methionine at position 79; a histidine or a phenylalanine at position 80; a glutamic acid at position 81; an alanine, a tyrosine, or a phenylalanine at position 83; an isoleucine at position 132; a valine at position 148; a leucine, a phenylalanine, or a tyrosine at position 162; an isoleucine at position 182; a leucine or tryptophan at position 202; a proline at position 204; an alanine, an arginine, a phenylalanine, a tyrosine, a histidine, or a tryptophan at position 208; and/or an isoleucine or a valine at position 213 of SEQ ID NO: 54.

12. The polypeptide of any one of the preceding claims, wherein the variant IgG Fc polypeptide comprises:
a) a phenylalanine or a tyrosine at a position corresponding to position 24, a threonine at a position corresponding to position 26, and/or a glutamic acid at a position corresponding to position 28 of SEQ ID NO: 1;
b) a phenylalanine at a position corresponding to position 24, a threonine at a position corresponding to position 26, and/or a glutamic acid at a position corresponding to position 28 of SEQ ID NO: 2, SEQ ID NO: 3, or SEQ ID NO: 4; or
c) a phenylalanine or a tyrosine at a position corresponding to position 24, a threonine at a position corresponding to position 26, and/or a glutamic acid at a position corresponding to position 28 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

13. The polypeptide of any one of the preceding claims, wherein the variant IgG Fc polypeptide comprises:
a) a phenylalanine or a tyrosine at position 24, a threonine at position 26, and/or a glutamic acid at position 28 of SEQ ID NO: 1;
b) a phenylalanine at position 24, a threonine at position 26, and/or a glutamic acid at position 28 of SEQ ID NO: 2, SEQ ID NO: 3, or SEQ ID NO: 4; or
c) a phenylalanine or a tyrosine at position 24, a threonine at position 26, and/or a glutamic acid at position 28 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

14. The polypeptide of any one of the preceding claims, wherein the variant IgG Fc polypeptide comprises a phenylalanine at a position corresponding to position 24 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

15. The polypeptide of any one of the preceding claims, wherein the variant IgG Fc polypeptide comprises a phenylalanine at position 24 of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

16. The polypeptide of any one of the preceding claims, wherein the variant IgG Fc polypeptide comprises:
a) a tyrosine at a position corresponding to position 24, a tyrosine at a position corresponding to position 83, and/or an arginine, a phenylalanine, a tyrosine, a histidine, or a tryptophan at a position corresponding to position 209 of SEQ ID NO: 1 or SEQ ID NO: 4;
b) a tyrosine at a position corresponding to position 24, a tyrosine at a position corresponding to position 83, and/or an arginine, a phenylalanine, a tyrosine, a histidine, or a tryptophan at a position corresponding to position 208 of SEQ ID NO: 2 or SEQ ID NO: 3; or
c) a tyrosine at a position corresponding to position 24, a tyrosine at a position corresponding to position 83, and/or an arginine, a phenylalanine, a tyrosine, a histidine, or a tryptophan at a position corresponding to position 208 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

17. The polypeptide of any one of the preceding claims, wherein the variant IgG Fc polypeptide comprises:
a) a tyrosine at position 24, a tyrosine at position 83, and/or an arginine, a phenylalanine, a tyrosine, a histidine, or a tryptophan at position 209 of SEQ ID NO: 1 or SEQ ID NO: 4;
b) a tyrosine at position 24, a tyrosine at position 83, and/or an arginine, a phenylalanine, a tyrosine, a histidine, or a tryptophan at position 208 of SEQ ID NO: 2 or SEQ ID NO: 3; or
c) a tyrosine at position 24, a tyrosine at position 83, and/or an arginine, a phenylalanine, a tyrosine, a histidine, or a tryptophan at position 208 of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

18. The polypeptide of any one of the preceding claims, wherein the variant IgG Fc polypeptide comprises:
a) a tyrosine at a position corresponding to position 24 and an arginine at a position corresponding to position 209 of SEQ ID NO: 1 or SEQ ID NO: 4;
b) a tyrosine at a position corresponding to position 24 and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
c) a tyrosine at a position corresponding to position 24 and a phenylalanine at a position corresponding to position 209 of SEQ ID NO: 1 or SEQ ID NO: 4;
d) a tyrosine at a position corresponding to position 24 and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
e) a tyrosine at a position corresponding to position 24 and a tyrosine at a position corresponding to position 209 of SEQ ID NO: 1 or SEQ ID NO: 4;
f) a tyrosine at a position corresponding to position 24 and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
g) a tyrosine at a position corresponding to position 24 and a histidine at a position corresponding to position 209 of SEQ ID NO: 1 or SEQ ID NO: 4;
h) a tyrosine at a position corresponding to position 24 and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
i) a tyrosine at a position corresponding to position 24 and a tryptophan at a position corresponding to position 209 of SEQ ID NO: 1 or SEQ ID NO: 4;
j) a tyrosine at a position corresponding to position 24 and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
k) a tyrosine at a position corresponding to position 83 and an arginine at a position corresponding to position 209 of SEQ ID NO: 1 or SEQ ID NO: 4;
l) a tyrosine at a position corresponding to position 83 and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
m) a tyrosine at a position corresponding to position 83 and a phenylalanine at a position corresponding to position 209 of SEQ ID NO: 1 or SEQ ID NO: 4;
n) a tyrosine at a position corresponding to position 83 and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
o) a tyrosine at a position corresponding to position 83 and a tyrosine at a position corresponding to position 209 of SEQ ID NO: 1 or SEQ ID NO: 4;
p) a tyrosine at a position corresponding to position 83 and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
q) a tyrosine at a position corresponding to position 83 and a histidine at a position corresponding to position 209 of SEQ ID NO: 1 or SEQ ID NO: 4;
r) a tyrosine at a position corresponding to position 83 and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
s) a tyrosine at a position corresponding to position 83 and a tryptophan at a position corresponding to position 209 of SEQ ID NO: 1 or SEQ ID NO: 4;
t) a tyrosine at a position corresponding to position 83 and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
u) a tyrosine at a position corresponding to position 24, a tyrosine at a position corresponding to position 83, and an arginine at a position corresponding to position 209 of SEQ ID NO: 1 or SEQ ID NO: 4;
v) a tyrosine at a position corresponding to position 24, a tyrosine at a position corresponding to position 83, and an arginine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
w) a tyrosine at a position corresponding to position 24, a tyrosine at a position corresponding to position 83, and a phenylalanine at a position corresponding to position 209 of SEQ ID NO: 1 or SEQ ID NO: 4;
x) a tyrosine at a position corresponding to position 24, a tyrosine at a position corresponding to position 83, and a phenylalanine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
y) a tyrosine at a position corresponding to position 24, a tyrosine at a position corresponding to position 83, and a tyrosine at a position corresponding to a position corresponding to position 209 of SEQ ID NO: 1 or SEQ ID NO: 4;
z) a tyrosine at a position corresponding to position 24, a tyrosine at a position corresponding to position 83, and a tyrosine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
aa) a tyrosine at a position corresponding to position 24, a tyrosine at a position corresponding to position 83, and a histidine at a position corresponding to position 209 of SEQ ID NO: 1 or SEQ ID NO: 4;
bb) a tyrosine at a position corresponding to position 24, a tyrosine at a position corresponding to position 83, and a histidine at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
cc) a tyrosine at a position corresponding to position 24, a tyrosine at a position corresponding to position 83, and a tryptophan at a position corresponding to position 209 of SEQ ID NO: 1 or SEQ ID NO: 4; or
dd) a tyrosine at a position corresponding to position 24, a tyrosine at a position corresponding to position 83, and a tryptophan at a position corresponding to position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

19. The polypeptide of any one of the preceding claims, wherein the variant IgG Fc polypeptide comprises:
a) a tyrosine at position 24 and an arginine at position 209 of SEQ ID NO: 1 or SEQ ID NO: 4;
b) a tyrosine at position 24 and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
c) a tyrosine at position 24 and a phenylalanine at position 209 of SEQ ID NO: 1 or SEQ ID NO: 4;
d) a tyrosine at position 24 and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
e) a tyrosine at position 24 and a tyrosine at position 209 of SEQ ID NO: 1 or SEQ ID NO: 4;
f) a tyrosine at position 24 and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
g) a tyrosine at position 24 and a histidine at position 209 of SEQ ID NO: 1 or SEQ ID NO: 4;
h) a tyrosine at position 24 and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
i) a tyrosine at position 24 and a tryptophan at position 209 of SEQ ID NO: 1 or SEQ ID NO: 4;
j) a tyrosine at position 24 and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
k) a tyrosine at position 83 and an arginine at position 209 of SEQ ID NO: 1 or SEQ ID NO: 4;
l) a tyrosine at position 83 and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
m) a tyrosine at position 83 and a phenylalanine at position 209 of SEQ ID NO: 1 or SEQ ID NO: 4;
n) a tyrosine at position 83 and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
o) a tyrosine at position 83 and a tyrosine at position 209 of SEQ ID NO: 1 or SEQ ID NO: 4;
p) a tyrosine at position 83 and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
q) a tyrosine at position 83 and a histidine at position 209 of SEQ ID NO: 1 or SEQ ID NO: 4;
r) a tyrosine at position 83 and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
s) a tyrosine at position 83 and a tryptophan at position 209 of SEQ ID NO: 1 or SEQ ID NO: 4;
t) a tyrosine at position 83 and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
u) a tyrosine at position 24, a tyrosine at position 83, and an arginine at position 209 of SEQ ID NO: 1 or SEQ ID NO: 4;
v) a tyrosine at position 24, a tyrosine at position 83, and an arginine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
w) a tyrosine at position 24, a tyrosine at position 83, and a phenylalanine at position 209 of SEQ ID NO: 1 or SEQ ID NO: 4;
x) a tyrosine at position 24, a tyrosine at position 83, and a phenylalanine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
y) a tyrosine at position 24, a tyrosine at position 83, and a tyrosine at position 209 of SEQ ID NO: 1 or SEQ ID NO: 4;
z) a tyrosine at position 24, a tyrosine at position 83, and a tyrosine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
aa) a tyrosine at position 24, a tyrosine at position 83, and a histidine at position 209 of SEQ ID NO: 1 or SEQ ID NO: 4;
bb) a tyrosine at position 24, a tyrosine at position 83, and a histidine at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54;
cc) a tyrosine at position 24, a tyrosine at position 83, and a tryptophan at position 209 of SEQ ID NO: 1 or SEQ ID NO: 4; or
dd) a tyrosine at position 24, a tyrosine at position 83, and a tryptophan at position 208 of SEQ ID NO: 2, SEQ ID NO: 3, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 54.

20. The polypeptide of any one of the preceding claims, wherein the variant IgG Fc polypeptide comprises the amino acid sequence of SEQ ID NO: 14, SEQ ID NO: 15, SEQ ID NO: 16, SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, or SEQ ID NO: 91.

21. The polypeptide of any one of the preceding claims, wherein the polypeptide comprises the amino acid sequence of SEQ ID NO: 14, SEQ ID NO: 15, SEQ ID NO: 16, SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, SEQ ID NO: 51, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, SEQ ID NO: 91, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 108, SEQ ID NO: 109, SEQ ID NO: 110, SEQ ID NO: 113, SEQ ID NO: 114, SEQ ID NO: 115, SEQ ID NO: 120, SEQ ID NO: 121, SEQ ID NO: 126, SEQ ID NO: 127, SEQ ID NO: 128, or SEQ ID NO: 129.

22. The polypeptide of any one of the preceding claims, wherein the at least one amino acid substitution comprises an amino acid substitution with an amino acid derivative.

23. The polypeptide of claim 22, wherein the amino acid derivative is a histidine derivative, a tryptophan derivative, a tyrosine derivative, a leucine derivative, a phenylalanine derivative, a valine derivative, a methionine derivative, an isoleucine derivative, an arginine derivative, a glutamic acid derivative, a proline derivative, a lysine derivative, a threonine derivative, an asparagine derivative, a glutamine derivative, a serine derivative, an alanine derivative, or a glutamic acid derivative.

24. The polypeptide of any one of the preceding claims, wherein the polypeptide is an antibody, an antibody fragment, or a fusion polypeptide.

25. The polypeptide of any one of the preceding claims, wherein the polypeptide comprises at least one therapeutic polypeptide selected from a late embryogenesis abundant (LEA) protein polypeptide (e.g., an LEA-3 polypeptide), an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ECD of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.

26. The polypeptide of claim 25, wherein the therapeutic polypeptide is a canine polypeptide, a feline polypeptide, or an equine polypeptide.

27. The polypeptide of any one of the preceding claims, wherein the polypeptide comprises an antibody or antibody fragment that binds at least one target polypeptide selected from a late embryogenesis abundant (LEA) protein polypeptide (e.g., an LEA-3 polypeptide), an NGF polypeptide, a receptor of an NGF polypeptide (e.g., an ECD of a receptor of an NGF polypeptide), a TrkA polypeptide (e.g., an ECD of a TrkA polypeptide), an LNGFR polypeptide (e.g., an ECD of a LNGFR polypeptide), a TNFα polypeptide, a receptor of a TNFα polypeptide, a TNFR polypeptide (e.g., an ECD of a TNFR polypeptide), a TNFR1 polypeptide (e.g., an ECD of a TNFR1 polypeptide), a TNFR2 polypeptide (e.g., an ECD of a TNFR2 polypeptide), an IL5 polypeptide, a receptor of an IL5 polypeptide, an IL5R polypeptide (e.g., an ECD of an IL5R polypeptide), an IL5Rα polypeptide (e.g., an ECD of an IL5Rα polypeptide), an IL6 polypeptide, a receptor of an IL6 polypeptide, an IL6R polypeptide (e.g., an ECD of an IL6R polypeptide), an IL17 polypeptide, a receptor of an IL17 polypeptide, an IL17R polypeptide (e.g., an ECD of an IL17R polypeptide), an IL17RA polypeptide (e.g. an ECD of an IL17RA polypeptide), an IL17RB polypeptide (e.g., an ECD of an IL17RB polypeptide), an IL17RC polypeptide (e.g., an ECD of an IL17RC polypeptide), an IL23 polypeptide, a receptor of an IL23 polypeptide, an IL23R polypeptide (e.g., an ECD of an IL23R polypeptide), an IL12Rβ1 polypeptide (e.g., an ECD of an IL12Rβ1 polypeptide), a PDL1 polypeptide, a receptor of a PDL1 polypeptide, a PDL2 polypeptide, a receptor of a PDL2 polypeptide, a PD1 polypeptide (e.g., an ECD of a PD1 polypeptide), an integrin polypeptide (e.g., ITGA1, ITGA2, ITGA3, ITGA4, ITGA5, ITGA6, ITGA7, ITGA8, ITGA9, ITGA10, ITGA11, ITGAD, ITGAE, ITGAL, ITGAM, ITGAV, ITGA2B, ITGAX, ITGB1, ITGB2, ITGB3, ITGB4, ITGB5, ITGB6, ITGB7, or ITGB8 polypeptide), a receptor of an integrin polypeptide, a fibronectin polypeptide (e.g., an ECD of a fibronectin polypeptide), a vitronectin polypeptide (e.g., an ECD of a vitronectin polypeptide), a collagen polypeptide (e.g., an ECD of a collagen polypeptide), a laminin polypeptide (e.g., an ECD of a laminin polypeptide), a CD80 polypeptide, a receptor of a CD80 polypeptide, a CD86 polypeptide, a receptor of a CD86 polypeptide, a CTLA-4 polypeptide (e.g., an ECD of a CTLA-4 polypeptide), a B7-H3 polypeptide, a receptor of a B7-H3 polypeptide (e.g., an ECD of receptor of a B7-H3 polypeptide), a LAG-3 polypeptide (e.g., an ECD of a LAG-3 polypeptide), an IL31 polypeptide, a receptor of an IL31 polypeptide, an IL31RA polypeptide (e.g., an ECD of an IL31RA polypeptide), an OSMR polypeptide (e.g., an ECD of an OSMR polypeptide), an IL4 polypeptide, a receptor of an IL4R polypeptide, an IL4R polypeptide (e.g., an ECD of an IL4R polypeptide), an IL13 polypeptide, a receptor of an IL13 polypeptide, an IL13RA1 polypeptide (e.g., an ECD of an IL13RA1 polypeptide), an IL4R polypeptide (e.g., an ECD of an IL4R polypeptide), an IL13Rα2 polypeptide (e.g., an ECD of an IL13Rα2 polypeptide), an IL22 polypeptide, a receptor of an IL22 polypeptide (e.g., an ECD of an IL22 polypeptide), an IL22Rα1 polypeptide (e.g., an ECD of an IL22Rα1 polypeptide), an IL10Rβ2 polypeptide (e.g., an ECD of an IL10Rβ2 polypeptide), an IL33 polypeptide, a receptor of an IL33 polypeptide, an IL1RL1 polypeptide (e.g., an ED of an IL1RL1 polypeptide), an EGF polypeptide, a receptor of an EGF polypeptide, a TGFα polypeptide, a receptor of a TGFα polypeptide, an EGFR polypeptide (e.g., an ECD of an EGFR polypeptide), an MMP9 polypeptide, an FGF polypeptide (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, or FGF23 polypeptide), a receptor of an FGF polypeptide, an FGFR polypeptide (e.g., FGFR1, FGFR2, FGFR3, FGFR4, or FGFRL1 polypeptide), an ECD of an FGFR polypeptide (e.g., an ECD of an FGFR1, an FGFR2, an FGFR3, an FGFR4, or an FGFRL1 polypeptide), an EGF polypeptide, a receptor of an EGF polypeptide, a neuregulin polypeptide (e.g., a neuregulin isoform I, II, III, IV, V, or VI polypeptide), a receptor of a neuregulin polypeptide, a HER polypeptide (e.g., HER1, HER2, HER3, or HER4 polypeptide), an ECD of a HER polypeptide (e.g., an ECD of a HER1, a HER2, a HER3, or a HER4 polypeptide), an EpCAM polypeptide (e.g., an ECD of an EpCAM polypeptide), a CD20 polypeptide (e.g., an ECD of a CD20 polypeptide), a ligand of a CD20 polypeptide, a CD19 polypeptide (e.g., an ECD of a CD19 polypeptide), a ligand of a CD19 polypeptide, a CGRP polypeptide (e.g., an α-CGRP polypeptide or a β-CGRP polypeptide), a receptor of a CGRP polypeptide, a receptor of an α-CGRP polypeptide, a receptor of a β-CGRP polypeptide, a CALCRL polypeptide (e.g., an ECD of a CALCRL polypeptide), a RAMP polypeptide (e.g., RAMP1, RAMP2, or RAMP3 polypeptide), an ECD of a RAMP polypeptide (e.g., an ECD of a RAMP1, RAMP2, or RAMP3 polypeptide), an IGF polypeptide (e.g., an IGF-1 or an IGF-2 polypeptide), a receptor of an IGF polypeptide (e.g., a receptor of an IGF-1 or an IGF-2 polypeptide), an IGFR polypeptide (e.g., an IGFR1 or an IGFR2 polypeptide), an ECD of an IGFR polypeptide (e.g., an ECD of an IGFR1 or an IGFR2 polypeptide), an IGFBP polypeptide (e.g., IGFBP1, IGFBP2, IGFBP3, IGFBP4, IGFBP5, or IGFBP6 polypeptide), a VEGF polypeptide (e.g., VEGF-A, VEGF-B, VEGF-C, VEGF-D, or PGF polypeptide), a receptor of a VEGF polypeptide (e.g., a receptor of a VEGF-A, a VEGF-B, a VEGF-C, a VEGF-D, or a PGF polypeptide), a VEGFR polypeptide (e.g., a VEGFR1, a VEGFR2, or a VEGFR3 polypeptide), an ECD of a VEGFR polypeptide (e.g., an ECD of a VEGFR1, a VEGFR2, or a VEGFR3 polypeptide), an FLT1 receptor polypeptide (e.g., an ECD of an FLT1 receptor polypeptide), an IL36 polypeptide (e.g., IL36A, IL36B, or IL36G polypeptide), a receptor of an IL36 polypeptide (e.g., a receptor of an IL36A, an IL36B, or an IL36G polypeptide), an IL36R polypeptide (e.g., an ECD of an IL36R polypeptide), an IL1R1 polypeptide (e.g., an ECD of an IL1R1 polypeptide), an IL1R2 polypeptide (e.g., an ECD of an IL1R2 polypeptide), an IL1RL1 polypeptide (an ECD of an IL1RL1 polypeptide), an IL18R1 polypeptide (an ECD of an IL18R1 polypeptide), a bacterial toxin polypeptide, an exotoxin polypeptide, an endotoxin polypeptide, a Botulinum neurotoxin polypeptide, a Tetanus toxin polypeptide, a Staphylococcal toxin polypeptide, a CD52 polypeptide (e.g., an ECD of a CD52 polypeptide), a ligand of a CD52 polypeptide, a SIGLEC10 polypeptide, a PCSK9 polypeptide, a receptor of a PCSK9 polypeptide, an LDLR polypeptide (e.g., an ECD of an LDLR polypeptide), a CEA polypeptide (e.g., CD66a, CD66b, CD66c, CD66d, CD66e, or CD66f polypeptide), an ECD of a CEA polypeptide (e.g., an ECD of a CD66a, a CD66b, a CD66c, a CD66d, a CD66e, or a CD66f polypeptide), a BAFF polypeptide, a receptor of a BAFF polypeptide, a TRAF polypeptide (e.g., TRAF1, TRAF2, TRAF3, TRAF4, TRAF5, TRAF6, TRAF7 polypeptide), a receptor of a TRAF polypeptide (e.g., a receptor of a TRAF1, a TRAF2, a TRAF3, a TRAF4, a TRAF5 polypeptide), a BCMA polypeptide, an ECD of a BCMA polypeptide, a SOST polypeptide, a receptor of a SOST polypeptide, an LRP polypeptide (e.g., an LRP5 or an LRP6 polypeptide), an ECD of an LRP polypeptide (e.g., an ECD of an LRP5 or an LRP6 polypeptide), a DLL polypeptide (e.g., a DLL4 polypeptide), a receptor of a DLL polypeptide, a Jagged polypeptide (e.g., JAG1 or JAG polypeptide), a receptor of a Jagged polypeptide (e.g., a receptor of a JAG1 or a JAG polypeptide), a NOTCH polypeptide (e.g., NOTCH1, NOTCH2, NOTCH3, or NOTCH4 polypeptide), a ligand of a NOTCH polypeptide (e.g., a ligand of a NOTCH1, a NOTCH2, a NOTCH3, or a NOTCH4 polypeptide), a VWF polypeptide, a receptor of a VWF polypeptide, a Factor VIII polypeptide, a receptor of a Factor VIII polypeptide, a platelet GP1b receptor polypeptide (e.g., an ECD of a platelet GP1b receptor polypeptide), an integrin α_{IIb}β₃ polypeptide (e.g., an ECD of an integrin α_{IIb}β₃ polypeptide), an IL2 polypeptide, a receptor of an IL2 polypeptide, an IL2R polypeptide (e.g., an IL2Rα, an IL2Rβ, or an IL2Rγ polypeptide), an ECD of an IL2R polypeptide (e.g., an ECD of an IL2Rα, an IL2Rβ, or an IL2Rγ polypeptide), a TGFβ polypeptide, a receptor of a TGFβ polypeptide, a Decorin polypeptide, an EIF3I polypeptide, a LTBP1 polypeptide, a TGFβR1 polypeptide (e.g., an ECD of a TGFβR1 polypeptide), a YWHAE polypeptide, an IgE polypeptide, a receptor or an IgE polypeptide, an Fc receptor polypeptide (e.g., an FcεRI or an FcεRII polypeptide), an ECD of an Fc receptor polypeptide (e.g., an ECD of an FcεRI or an FcεRII polypeptide), a KLK polypeptide (e.g., KLK1, KLK2, KLK3, KLK4, KLK5, KLK6, KLK7, KLK8, KLK9, KLK10, KLK11, KLK12, KLK13, KLK14, or KLK15 polypeptide), a Rankl polypeptide, a receptor of a Rankl polypeptide, a RANK polypeptide (e.g., an ECD of a RANK polypeptide), a TSLP polypeptide, a receptor of a TSLP polypeptide, a CRLF2 polypeptide (e.g., an ECD of a CRLF2 polypeptide), an IL7Rα polypeptide (e.g., an ECD of an IL7Rα polypeptide), an S1P polypeptide, a CD3 polypeptide (e.g., a CD3γ polypeptide, a CD3δ polypeptide, or a CD3ε polypeptide), an ECD of a CD3 polypeptide (e.g., an ECD of a CD3γ polypeptide, a CD3δ polypeptide, or a CD3ε polypeptide), a CD80 polypeptide, a receptor of a CD80 polypeptide, a CD28 polypeptide (e.g., an ECD of a CD28 polypeptide), a CTLA-4 polypeptide (e.g., an ECD of a CTLA-4 polypeptide), a GnRH polypeptide, a receptor of a GNRH polypeptide, a GnRHR polypeptide (e.g., an ECD of a GnRHR polypeptide), an ICAM polypeptide (e.g., ICAM-1, ICAM-2, ICAM-3, ICAM-4, or ICAM-5 polypeptide), a receptor of an ICAM polypeptide (e.g., a receptor of an ICAM-1, an ICAM-2, an ICAM-3, an ICAM-4, or an ICAM-5 polypeptide), a JAM-A polypeptide, a receptor of a JAM-A polypeptide, an LFA-1 polypeptide (e.g., an ECD of an LFA-1 polypeptide), a Naᵥ1.7 polypeptide, a C5 polypeptide (e.g., a C5a or a C5b polypeptide), a receptor of a C5 polypeptide (e.g., a receptor of a C5a or a C5b polypeptide), a C5aR polypeptide (e.g., an ECD of a C5aR polypeptide), a C5L2 polypeptide (e.g., an ECD of a C5L2 polypeptide), an IL17 polypeptide, a receptor of an IL17 polypeptide, an IL17Ra polypeptide (e.g., an ECD of an IL17Ra polypeptide), an IL17RC polypeptide (e.g., an ECD of an IL17RC polypeptide), an EPO polypeptide, a somatostatin polypeptide, a GLP1 polypeptide, and a glucagon polypeptide.

28. The polypeptide of claim 27, wherein the target polypeptide is a canine polypeptide, a feline polypeptide, or an equine polypeptide.

29. The polypeptide of any one of claims 1 to 24, wherein the polypeptide comprises an antibody or antibody fragment that binds canine and/or feline IL31.

30. The polypeptide of any one of the preceding claims, wherein the variant IgG Fc polypeptide has altered binding affinity to Protein A, C1q, CD16, CD32, or CD64 relative to the wild-type IgG Fc polypeptide.

31. A polypeptide comprising the amino acid sequence of SEQ ID NO: 14, SEQ ID NO: 15, SEQ ID NO: 16, SEQ ID NO: 17, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 50, SEQ ID NO: 51, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 85, SEQ ID NO: 87, SEQ ID NO: 89, SEQ ID NO: 91, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 108, SEQ ID NO: 109, SEQ ID NO: 110, SEQ ID NO: 113, SEQ ID NO: 114, SEQ ID NO: 115, SEQ ID NO: 120, SEQ ID NO: 121, SEQ ID NO: 126, SEQ ID NO: 127, SEQ ID NO: 128, or SEQ ID NO: 129.

32. An isolated nucleic acid encoding the polypeptide of any one of the preceding claims.

33. A host cell comprising the nucleic acid of claim 32.

34. A host cell that expresses the polypeptide of any one of claims 1 to 33.

35. A method of producing a polypeptide comprising culturing the host cell of claim 33 or of claim 34 and isolating the polypeptide.

36. A pharmaceutical composition comprising the polypeptide of any one of claims 1 to 31 and a pharmaceutically acceptable carrier.

37. A method of delivering a polypeptide to a companion animal species comprising administering the polypeptide of any one of claims 1 to 31 or the pharmaceutical composition of claim 36 parenterally.

38. A method of delivering a polypeptide to a companion animal species comprising administering the polypeptide of any one of claims 1 to 31 or the pharmaceutical composition of claim 36 by an intramuscular route, an intraperitoneal route, an intracerebrospinal route, a subcutaneous route, an intra-arterial route, an intrasynovial route, an intrathecal route, or an inhalation route.

39. The method of claim 37 or claim 38, wherein the companion animal species is canine, equine, or feline.


SEQUENCE LISTING (134 sequences)

SEQ ID NO: 1 - protein, 222 aa, Canis lupus familiaris
  misc_feature (1)..(222): Exemplary wild-type canine IgG-A Fc
CPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 2 - protein, 221 aa, Canis lupus familiaris
  misc_feature (1)..(221): Exemplary wild-type canine IgG-B Fc
CPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 3 - protein, 221 aa, Canis lupus familiaris
  misc_feature (1)..(221): Exemplary wild-type canine IgG-C Fc
CPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 4 - protein, 222 aa, Canis lupus familiaris
  misc_feature (1)..(222): Exemplary wild-type canine IgG-D Fc
CPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 5 - protein, 221 aa, Equus caballus
  misc_feature (1)..(221): Exemplary wild-type equine IgG1 Fc
CPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 6 - protein, 221 aa, Equus caballus
  misc_feature (1)..(221): Exemplary wild-type equine IgG2 Fc
YTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 7 - protein, 221 aa, Equus caballus
  misc_feature (1)..(221): Exemplary wild-type equine IgG3 Fc
CPAPELLGGPSVFIFPPKPKDVLMITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 8 - protein, 221 aa, Equus caballus
  misc_feature (1)..(221): Exemplary wild-type equine IgG4 Fc
CPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 9 - protein, 221 aa, Equus caballus
  misc_feature (1)..(221): Exemplary wild-type equine IgG5 Fc
CPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 10 - protein, 221 aa, Equus caballus
  misc_feature (1)..(221): Exemplary wild-type equine IgG6 Fc
CCCPKCPGRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 11 - protein, 221 aa, Equus caballus
  misc_feature (1)..(221): Exemplary wild-type equine IgG7 Fc
TCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 12 - protein, 221 aa, Felis catus
  misc_feature (1)..(221): Exemplary wild-type feline IgG1a Fc
CPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 13 - protein, 221 aa, Felis catus
  misc_feature (1)..(221): Exemplary wild-type feline IgG1b Fc
CPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 14 - protein, 221 aa, Artificial Sequence
  Synthetic Exemplary variant canine IgG-B Fc (Y00)  Leu24Tyr
CPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 15 - protein, 221 aa, Artificial Sequence
  Synthetic Exemplary variant canine IgG-B Fc (F00)  Leu24Phe
CPAPEMLGGPSVFIFPPKPKDTLFIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 16 - protein, 221 aa, Artificial Sequence
  Synthetic Exemplary variant canine IgG-B Fc (M00)  Leu24Met
CPAPEMLGGPSVFIFPPKPKDTLMIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 17 - protein, 221 aa, Artificial Sequence
  Synthetic Exemplary variant canine IgG-B Fc (S00)  Leu24Ser
CPAPEMLGGPSVFIFPPKPKDTLSIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 18 - protein, 305 aa, Artificial Sequence
  Synthetic Exemplary canine FcRn with poly-His
MGVPRPRSWGLGFLLFLLPTLRAADSHLSLLYHLTAVSAPPPGTPAFWASGWLGPQQYLSYNNLRAQAEPYGAWVWENQVSWYWEKETTDLRTKEGLFLEALKALGDGGPYTLQGLLGCELGPDNTSVPVAKFALNGEDFMTFDPKLGTWNGDWPETETVSKRWMQQAGAVSKERTFLLYSCPQRLLGHLERGRGNLEWKEPPSMRLKARPGSPGFSVLTCSAFSFYPPELQLRFLRNGLAAGSGEGDFGPNGDGSFHAWSSLTVKSGDEHHYRCLVQHAGLPQPLTVELESPAKSSGSHHHHHH

SEQ ID NO: 19 - protein, 269 aa, Artificial Sequence
  Synthetic Exemplary canine FcRn ECD
HLSLLYHLTAVSAPPPGTPAFWASGWLGPQQYLSYNNLRAQAEPYGAWVWENQVSWYWEKETTDLRTKEGLFLEALKALGDGGPYTLQGLLGCELGPDNTSVPVAKFALNGEDFMTFDPKLGTWNGDWPETETVSKRWMQQAGAVSKERTFLLYSCPQRLLGHLERGRGNLEWKEPPSMRLKARPGSPGFSVLTCSAFSFYPPELQLRFLRNGLAAGSGEGDFGPNGDGSFHAWSSLTVKSGDEHHYRCLVQHAGLPQPLTVELESPAK

SEQ ID NO: 20 - protein, 125 aa, Artificial Sequence
  Synthetic Exemplary canine B2M
MAPRPALATAGFLALLLILLAACRLDAVQHPPKIQVYSRHPAENGKPNFLNCYVSGFHPPEIEIDLLKNGKEMKAEQTDLSFSKDWTFYLLVHTEFTPNEQDEFSCRVKHVTLSEPQIVKWDRDN

SEQ ID NO: 21 - protein, 305 aa, Artificial Sequence
  Synthetic Exemplary feline FcRn with poly-His
MGVPRPQPWGLGFLLFLLPTLRAAESHLSLLYHLTAVSSPAPGTPAFWVSGWLGPQQYLSYNNMRAQAEPCGAWVWENQVSWYWEKETTDLRNKQELFLEALKVLGEGGPYTLQGLLGCELGPDNASVPVAKFALNGEDFMDFDPKLGTWSGEWPETETISKRWMQEAGAVSKERTFLLNSCPQRLLGHLERGRGNLEWKEPPSMRLKARPGSPGFSVLTCSAFSFYPPELQLRFLRNGLAAGSGEGDFGPNGDGSFHAWSSLTVKSGDEHHYRCLVQHAGLPQPLTVELESPAKSSGSHHHHHH

SEQ ID NO: 22 - protein, 269 aa, Artificial Sequence
  Synthetic Exemplary feline FcRn ECD
HLSLLYHLTAVSSPAPGTPAFWVSGWLGPQQYLSYNNMRAQAEPCGAWVWENQVSWYWEKETTDLRNKQELFLEALKVLGEGGPYTLQGLLGCELGPDNASVPVAKFALNGEDFMDFDPKLGTWSGEWPETETISKRWMQEAGAVSKERTFLLNSCPQRLLGHLERGRGNLEWKEPPSMRLKARPGSPGFSVLTCSAFSFYPPELQLRFLRNGLAAGSGEGDFGPNGDGSFHAWSSLTVKSGDEHHYRCLVQHAGLPQPLTVELESPAK

SEQ ID NO: 23 - protein, 118 aa, Artificial Sequence
  Synthetic Exemplary feline B2M
MARFVVLVLLGLLYLSHLDAVQHSPKVQVYSRHPAENGKPNFLNCYVSGFHPPQIDITLMKNGKKMEAEQTDLSFNRDWTFYLLVHTEFTPTVEDEYSCQVNHTTLSEPKVVKWDRDM

SEQ ID NO: 24 - protein, 407 aa, Artificial Sequence
  Synthetic Exemplary equine FcRn
PPDSGSVCLTLGLPLPLWICPLVCGSVLLCLGRSPPPPQVCPVSLGLSLLSLSFCLSGSPSSPHPPPALFTRFSPAESRPSLLYHFTAVSSPAPGAPAFWVSGWLGPQQYLSYNNLRAQAEPCGAWVWENQVSWYWEKETTDLRKKEKLFLDAFQVLTEEGPHTLQGLLGCELNPDNTSVPVAKFALDGEDFMEFDLKLGAWNGDWPEALAIGQRWARQADTTKMCRSSLLYSCPERLLRERERRRENLEWKEPPSMRLKARPGSPGFSVLTCSAFSFYPPELQLRFLRNGLAAGSGEGDLGPNGDGSFYAWSSLTVKSGDEYHYRCWVQHAGLAQPLTVELESPAKSPMQVVGIVVGVLLLLVLAAGGALLWWRMRKGLPAPWISLRGDDIGALLPAPGLPKDADS

SEQ ID NO: 25 - protein, 266 aa, Artificial Sequence
  Synthetic Exemplary equine FcRn ECD
LLYHFTAVSSPAPGAPAFWVSGWLGPQQYLSYNNLRAQAEPCGAWVWENQVSWYWEKETTDLRKKEKLFLDAFQVLTEEGPHTLQGLLGCELNPDNTSVPVAKFALDGEDFMEFDLKLGAWNGDWPEALAIGQRWARQADTTKMCRSSLLYSCPERLLRERERRRENLEWKEPPSMRLKARPGSPGFSVLTCSAFSFYPPELQLRFLRNGLAAGSGEGDLGPNGDGSFYAWSSLTVKSGDEYHYRCWVQHAGLAQPLTVELESPAK

SEQ ID NO: 26 - protein, 118 aa, Artificial Sequence
  Synthetic Exemplary equine  B2M
MARVVALVLLGLLSLTGLEAVPRVPKVQVYSRHPAENGKPNFLNCYVSGFHPPEIEIDLLKNGEKMKVDRSDLSFSKDWSFYLLVHTDFTPNGVDEYSCRVQHSTLKDPLIVKWDRDL

SEQ ID NO: 27 - protein, 221 aa, Artificial Sequence
  Synthetic Exemplary variant canine IgG-B Fc (0Y0)  Gln83Tyr
CPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 28 - protein, 221 aa, Artificial Sequence
  Synthetic Exemplary variant canine IgG-B Fc (00R)  Asn208Arg
CPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHRHYTQESLSHSPGK

SEQ ID NO: 29 - protein, 221 aa, Artificial Sequence
  Synthetic Exemplary variant canine IgG-B Fc (00F)  Asn208Phe
CPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHFHYTQESLSHSPGK

SEQ ID NO: 30 - protein, 221 aa, Artificial Sequence
  Synthetic Exemplary variant canine IgG-B Fc (00Y)  Asn208Tyr
CPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 31 - protein, 221 aa, Artificial Sequence
  Synthetic Exemplary variant canine IgG-B Fc (00H)  Asn208His
CPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 32 - protein, 221 aa, Artificial Sequence
  Synthetic Exemplary variant canine IgG-B Fc (00W)  Asn208Trp
CPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHWHYTQESLSHSPGK

SEQ ID NO: 33 - protein, 221 aa, Artificial Sequence
  Synthetic Exemplary variant canine IgG-B Fc (Y0R)  Leu24Tyr Asn208Arg
CPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHRHYTQESLSHSPGK

SEQ ID NO: 34 - protein, 221 aa, Artificial Sequence
  Synthetic Exemplary variant canine IgG-B Fc (Y0F)  Leu24Tyr Asn208Phe
CPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHFHYTQESLSHSPGK

SEQ ID NO: 35 - protein, 221 aa, Artificial Sequence
  Synthetic Exemplary variant canine IgG-B Fc (Y0Y)  Leu24Tyr Asn208Tyr
CPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 36 - protein, 221 aa, Artificial Sequence
  Synthetic Exemplary variant canine IgG-B Fc (Y0H)  Leu24Tyr Asn208His
CPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 37 - protein, 221 aa, Artificial Sequence
  Synthetic Exemplary variant canine IgG-B Fc (Y0W)  Leu24Tyr Asn208Trp
CPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHWHYTQESLSHSPGK

SEQ ID NO: 38 - protein, 221 aa, Artificial Sequence
  Synthetic Exemplary variant canine IgG-B Fc (0YR)  Gln83Tyr Asn208Arg
CPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHRHYTQESLSHSPGK

SEQ ID NO: 39 - protein, 221 aa, Artificial Sequence
  Synthetic Exemplary variant canine IgG-B Fc (0YF)  Gln83Tyr Asn208Phe
CPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHFHYTQESLSHSPGK

SEQ ID NO: 40 - protein, 221 aa, Artificial Sequence
  Synthetic Exemplary variant canine IgG-B Fc (0YY)  Gln83Tyr Asn208Tyr
CPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 41 - protein, 221 aa, Artificial Sequence
  Synthetic Exemplary variant canine IgG-B Fc (0YH)  Gln83Tyr Asn208His
CPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 42 - protein, 221 aa, Artificial Sequence
  Synthetic Exemplary variant canine IgG-B Fc (0YW)  Gln83Tyr Asn208Trp
CPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHWHYTQESLSHSPGK

SEQ ID NO: 43 - protein, 221 aa, Artificial Sequence
  Synthetic Exemplary variant canine IgG-B Fc (YYR)  Leu24Tyr Gln83Tyr Asn208Arg
CPAPEMLGGPSVFIFPPKPKDTLYIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHRHYTQESLSHSPGK

SEQ ID NO: 44 - protein, 221 aa, Artificial Sequence
  Synthetic Exemplary variant canine IgG-B Fc (YYF)  Leu24Tyr Gln83Tyr Asn208Phe
CPAPEMLGGPSVFIFPPKPKDTLYIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHFHYTQESLSHSPGK

SEQ ID NO: 45 - protein, 221 aa, Artificial Sequence
  Synthetic Exemplary variant canine IgG-B Fc (YYY)  Leu24Tyr Gln83Tyr Asn208Tyr
CPAPEMLGGPSVFIFPPKPKDTLYIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 46 - protein, 221 aa, Artificial Sequence
  Synthetic Exemplary variant canine IgG-B Fc (YYH)  Leu24Tyr Gln83Tyr Asn208His
CPAPEMLGGPSVFIFPPKPKDTLYIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 47 - protein, 221 aa, Artificial Sequence
  Synthetic Exemplary variant canine IgG-B Fc (YYW)  Leu24Tyr Gln83Tyr Asn208Trp
CPAPEMLGGPSVFIFPPKPKDTLYIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHWHYTQESLSHSPGK

SEQ ID NO: 48 - protein, 221 aa, Artificial Sequence
  Synthetic Exemplary variant canine IgG-B Fc (YTE)  Leu24Tyr Ala26Thr Thr28Glu
CPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 49 - protein, 453 aa, Artificial Sequence
  Synthetic Exemplary caninized variable heavy chain of mouse anti-canine IL31 clone M14 and canine IgG-B
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 50 - protein, 453 aa, Artificial Sequence
  Synthetic Exemplary caninized variable heavy chain of mouse anti-canine IL31 clone M14 and YYY variant canine IgG-B Leu24Tyr Gln83Tyr  Asn208Tyr (amino acid substitution numbering corresponding to position of canine IgGB Fc (SEQ ID NO 2))
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 51 - protein, 453 aa, Artificial Sequence
  Synthetic Exemplary caninized variable heavy chain of mouse anti-canine IL31 clone M14 and Y0Y variant canine IgG-B  Leu24Tyr Asn208Tyr (amino acid substitution numbering corresponding to position of canine IgGB Fc (SEQ ID NO 2))
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 52 - protein, 453 aa, Artificial Sequence
  Synthetic Exemplary caninized variable heavy chain of mouse anti-canine IL31 clone M14 and YTE variant canine IgG-B  Leu24Tyr Ala26Thr Thr28Glu (amino acid substitution numbering corresponding to position of canine IgGB Fc (SEQ ID NO 2))
EVQLVESGPSLVKPGGSLRLTCSVTGDSITSGYWNWIRKFPGNKLEYMGYISYSGITDYNPSLKSRITISRDTSKNQYYLQLNSVTTEDTATYYCARYGNYGYAMDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 53 - protein, 221 aa, Artificial Sequence
  Synthetic Exemplary caninized light chain of mouse anticanine IL31 clone M14 and canine kappa light chain constant region
DIVMTQSPASLSVSLGQRATISCRASESVDTYGNSFMHWYQQKPGQSPKLLIYRASNLESGIPARFGGSGSGTDFTLTIDPVQADDVATYYCQQSYEDPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 54 - protein, 221 aa, Felis catus
  misc_feature (1)..(221): Exemplary wild-type feline IgG2 Fc
CPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 55 - protein, 222 aa, Artificial Sequence
  Synthetic Exemplary variant canine IgG-A Fc (F00)  Arg24Phe
CPVPEPLGGPSVLIFPPKPKDILFITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 56 - protein, 221 aa, Artificial Sequence
  Synthetic Exemplary variant canine IgG-C Fc (F00)  Val24Phe
CPGCGLLGGPSVFIFPPKPKDILFTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 57 - protein, 222 aa, Artificial Sequence
  Synthetic Exemplary variant canine IgG-D Fc (F00)  Arg24Phe
CPVPESLGGPSVFIFPPKPKDILFITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 58 - protein, 222 aa, Artificial Sequence
  Synthetic Exemplary variant canine IgG-A Fc (Y00)  Arg24Tyr
CPVPEPLGGPSVLIFPPKPKDILYITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 59 - protein, 221 aa, Artificial Sequence
  Synthetic Exemplary variant canine IgG-C Fc (Y00)  Val24Tyr
CPGCGLLGGPSVFIFPPKPKDILYTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 60 - protein, 222 aa, Artificial Sequence
  Synthetic Exemplary variant canine IgG-D Fc (Y00)  Arg24Tyr
CPVPESLGGPSVFIFPPKPKDILYITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 61 - protein, 221 aa, Artificial Sequence
  Synthetic Exemplary variant equine IgG1 Fc (F00)  Met24Phe
CPAPELLGGPSVFIFPPNPKDTLFITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 62 - protein, 221 aa, Artificial Sequence
  Synthetic Exemplary variant equine IgG2 Fc (F00)  Met24Phe
YTHSKFLGGPSVFIFPPNPKDALFISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 63 - protein, 221 aa, Artificial Sequence
  Synthetic Exemplary variant equine IgG3 Fc (F00)  Met24Phe
CPAPELLGGPSVFIFPPKPKDVLFITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 64 - protein, 221 aa, Artificial Sequence
  Synthetic Exemplary variant equine IgG4 Fc (F00)  Met24Phe
CPAECLQVGPSVFIFPPKPKDVLFISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 65 - protein, 221 aa, Artificial Sequence
  Synthetic Exemplary variant equine IgG5 Fc (F00)  Lys24Phe
CPAPELPGGPSVFIFPPKPKDVLFISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 66 - protein, 221 aa, Artificial Sequence
  Synthetic Exemplary variant equine IgG6 Fc (F00)  Met24Phe
CCCPKCPGRPSVFIFPPNPKDTLFISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 67 - protein, 221 aa, Artificial Sequence
  Synthetic Exemplary variant equine IgG7 Fc (F00)  Met24Phe
TCPECLSVGPSVFIFPPKPKDVLFISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 68 - protein, 221 aa, Artificial Sequence
  Synthetic Exemplary variant equine IgG1 Fc (Y00)  Met24Tyr
CPAPELLGGPSVFIFPPNPKDTLYITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 69 - protein, 221 aa, Artificial Sequence
  Synthetic Exemplary variant equine IgG2 Fc (Y00)  Met24Tyr
YTHSKFLGGPSVFIFPPNPKDALYISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 70 - protein, 221 aa, Artificial Sequence
  Synthetic Exemplary variant equine IgG3 Fc (Y00)  Met24Tyr
CPAPELLGGPSVFIFPPKPKDVLYITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 71 - protein, 221 aa, Artificial Sequence
  Synthetic Exemplary variant equine IgG4 Fc (Y00)  Met24Tyr
CPAECLQVGPSVFIFPPKPKDVLYISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 72 - protein, 221 aa, Artificial Sequence
  Synthetic Exemplary variant equine IgG5 Fc (Y00)  Lys24Tyr
CPAPELPGGPSVFIFPPKPKDVLYISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 73 - protein, 221 aa, Artificial Sequence
  Synthetic Exemplary variant equine IgG6 Fc (Y00)  Met24Tyr
CCCPKCPGRPSVFIFPPNPKDTLYISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 74 - protein, 221 aa, Artificial Sequence
  Synthetic Exemplary variant equine IgG7 Fc (Y00)  Met24Tyr
TCPECLSVGPSVFIFPPKPKDVLYISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 75 - protein, 221 aa, Artificial Sequence
  Synthetic Exemplary variant feline IgG1a Fc (F00)  Ser24Phe
CPPPEMLGGPSIFIFPPKPKDTLFISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 76 - protein, 221 aa, Artificial Sequence
  Synthetic Exemplary variant feline IgG1b Fc (F00)  Ser24Phe
CPPPEMLGGPSIFIFPPKPKDTLFISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 77 - protein, 221 aa, Artificial Sequence
  Synthetic Exemplary variant feline IgG2 Fc (F00)  Ser24Phe
CPVPEIPGAPSVFIFPPKPKDTLFISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 78 - protein, 221 aa, Artificial Sequence
  Synthetic Exemplary variant feline IgG1a Fc (Y00)  Ser24Tyr
CPPPEMLGGPSIFIFPPKPKDTLTISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 79 - protein, 221 aa, Artificial Sequence
  Synthetic Exemplary variant feline IgG1a Fc (Y00)  Ser24Tyr
CPPPEMLGGPSIFIFPPKPKDTLTISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 80 - protein, 221 aa, Artificial Sequence
  Synthetic Exemplary variant feline IgG2 Fc (Y00)  Ser24Tyr
CPVPEIPGAPSVFIFPPKPKDTLTISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 81 - protein, 221 aa, Artificial Sequence
  Synthetic Exemplary variant feline IgG1a Fc (YTE)  Ser24Tyr Ser26Thr Thr28Glu
CPPPEMLGGPSIFIFPPKPKDTLYITREPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 82 - protein, 221 aa, Artificial Sequence
  Synthetic Exemplary variant feline IgG1b Fc (YTE)  Ser24Tyr Ser26Thr Thr28Glu
CPPPEMLGGPSIFIFPPKPKDTLYITREPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 83 - protein, 221 aa, Artificial Sequence
  Synthetic Exemplary variant feline IgG2 Fc (YTE)  Ser24Tyr Ser26Thr Thr28Glu
CPVPEIPGAPSVFIFPPKPKDTLYITREPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 84 - protein, 222 aa, Artificial Sequence
  Synthetic Exemplary variant canine IgG-A Fc (Protein A+; C1q; CD16 )  I(22)T; R(24)L; T(26)A; E(81)G; T(206)A; Q(208)H
CPVPEPLGGPSVLIFPPKPKDTLLIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 85 - protein, 222 aa, Artificial Sequence
  Synthetic Exemplary variant canine IgG-A Fc (F00; Protein A+; C1q; CD16 )  I(22)T; R(24)F; T(26)A; E(81)G; T(206)A; Q(208)H
CPVPEPLGGPSVLIFPPKPKDTLFIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 86 - protein, 222 aa, Artificial Sequence
  Synthetic Exemplary variant canine IgG-A Fc (Protein A+; C1q+; CD16 +)  V3A; P6M; I22T; R24L; T26A; L36V; G39D; R40P; Q66E; E81G; R94K; H97N; I98K; D99A; T206A; Q208H
CPAPEMLGGPSVLIFPPKPKDTLLIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 87 - protein, 222 aa, Artificial Sequence
  Synthetic Exemplary variant canine IgG-A Fc (F00; Protein A+; C1q+; CD16 +)  V3A; P6M; I22T; R24F; T26A; L36V; G39D; R40P; Q66E; E81G; R94K; H97N; I98K; D99A; T206A; Q208H
CPAPEMLGGPSVLIFPPKPKDTLFIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 88 - protein, 222 aa, Artificial Sequence
  Synthetic Exemplary variant canine IgG-D Fc (Protein A+; C1q; CD16 )  I(22)T; R(24)L; T(26)A; E(81)G; Q(208)H
CPVPESLGGPSVFIFPPKPKDTLLIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 89 - protein, 222 aa, Artificial Sequence
  Synthetic Exemplary variant canine IgG-D Fc (F00; Protein A+; C1q; CD16 )  I(22)T; R(24)F; T(26)A; E(81)G; Q(206)A; Q(208)H
CPVPESLGGPSVFIFPPKPKDTLFIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 90 - protein, 222 aa, Artificial Sequence
  Synthetic Exemplary variant canine IgG-D Fc (Protein A+; C1q+; CD16 +)  V3A; S6M; I22T; R24L; T26A; L36V; G39D; R40P; Q66E; E81G; R94K; H97N; I98K; G99A; Q208H
CPAPEMLGGPSVFIFPPKPKDTLLIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 91 - protein, 222 aa, Artificial Sequence
  Synthetic Exemplary variant canine IgG-D Fc (F00; Protein A+; C1q+; CD16 +)  V3A; S6M; I22T; R24L; T26A; L36V; G39D; R40P; Q66E; E81G; R94K; H97N; I98K; G99A; Q208H
CPAPEMLGGPSVFIFPPKPKDTLFIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 92 - protein, 237 aa, Artificial Sequence
  Synthetic Exemplary chimeric D2E7 antiTNF- alpha variable light chain and canine kappa constant light chain with leader sequence
METDTLLLWVLLLWVPGSTGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 93 - protein, 217 aa, Artificial Sequence
  Synthetic Exemplary chimeric D2E7 antiTNF- alpha variable light chain and canine kappa constant light chain without leader sequence
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 94 - protein, 475 aa, Artificial Sequence
  Synthetic Exemplary chimeric D2E7 antiTNF- alpha variable heavy chain and wild-type canine IgG-B Fc with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 95 - protein, 456 aa, Artificial Sequence
  Synthetic Exemplary chimeric D2E7 antiTNF- alpha variable heavy chain and wild-type canine IgG-B Fc without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 96 - protein, 475 aa, Artificial Sequence
  Synthetic Exemplary chimeric D2E7 antiTNF- alpha variable heavy chain and variant canine IgG-B Fc (0Y0) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 97 - protein, 456 aa, Artificial Sequence
  Synthetic Exemplary chimeric D2E7 antiTNF- alpha variable heavy chain and variant canine IgG-B Fc (0Y0) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 98 - protein, 475 aa, Artificial Sequence
  Synthetic Exemplary chimeric D2E7 antiTNF- alpha variable heavy chain and variant canine IgG-B Fc (0YH) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 99 - protein, 456 aa, Artificial Sequence
  Synthetic Exemplary chimeric D2E7 antiTNF- alpha variable heavy chain and variant canine IgG-B Fc (0YH) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 100 - protein, 475 aa, Artificial Sequence
  Synthetic Exemplary chimeric D2E7 antiTNF- alpha variable heavy chain and variant canine IgG-B Fc (0YY) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 101 - protein, 456 aa, Artificial Sequence
  Synthetic Exemplary chimeric D2E7 antiTNF- alpha variable heavy chain and variant canine IgG-B Fc (0YY) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHYDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 102 - protein, 475 aa, Artificial Sequence
  Synthetic Exemplary chimeric D2E7 antiTNF- alpha variable heavy chain and variant canine IgG-B Fc (00Y) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 103 - protein, 456 aa, Artificial Sequence
  Synthetic Exemplary chimeric D2E7 antiTNF- alpha variable heavy chain and variant canine IgG-B Fc (00Y) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHYHYTQESLSHSPGK

SEQ ID NO: 104 - protein, 475 aa, Artificial Sequence
  Synthetic Exemplary chimeric D2E7 antiTNF- alpha variable heavy chain and variant canine IgG-B Fc (F00) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLFIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 105 - protein, 456 aa, Artificial Sequence
  Synthetic Exemplary chimeric D2E7 antiTNF- alpha variable heavy chain and variant canine IgG-B Fc (F00) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLFIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 106 - protein, 471 aa, Artificial Sequence
  Synthetic Exemplary chimeric D2E7 antiTNF- alpha variable heavy chain and variable canine IgG-A Fc (Protein A+; C1q+; CD16+) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPAPEMLGGPSVLIFPPKPKDTLLIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 107 - protein, 452 aa, Artificial Sequence
  Synthetic Exemplary chimeric D2E7 antiTNF- alpha variable heavy chain and variable canine IgG-A Fc (Protein A+; C1q+; CD16+) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPAPEMLGGPSVLIFPPKPKDTLLIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 108 - protein, 452 aa, Artificial Sequence
  Synthetic Exemplary chimeric D2E7 antiTNF- alpha variable heavy chain and variable canine IgG-A Fc (F00; Protein A+; C1q+; CD16+) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPAPEMLGGPSVLIFPPKPKDTLFIARTPEVTCVVVDLDPEDPEVQISWFVDGKEVHTAKTQSREEQFNGTYRVVSVLPIGHQDWLTGKEFKCKVNNKALPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 109 - protein, 471 aa, Artificial Sequence
  Synthetic Exemplary chimeric D2E7 antiTNF- alpha variable heavy chain and variable canine IgG-A Fc (F00, Protein A+; C1q; CD16) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDTLFIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 110 - protein, 452 aa, Artificial Sequence
  Synthetic Exemplary chimeric D2E7 antiTNF- alpha variable heavy chain and variable canine IgG-A Fc (F00, Protein A+; C1q; CD16) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDTLFIARTPEVTCVVLDLGREDPEVQISWFVDGKEVHTAKTQSREQQFNGTYRVVSVLPIGHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHEALHNHYTDLSLSHSPGK

SEQ ID NO: 111 - protein, 471 aa, Artificial Sequence
  Synthetic Exemplary chimeric D2E7 antiTNF- alpha variable heavy chain and variable canine IgG-D Fc (Protein A+; C1q+; CD16+) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPAPEMLGGPSVFIFPPKPKDTLLIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 112 - protein, 452 aa, Artificial Sequence
  Synthetic Exemplary chimeric D2E7 antiTNF- alpha variable heavy chain and variable canine IgG-D Fc (Protein A+; C1q+; CD16+) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPAPEMLGGPSVFIFPPKPKDTLLIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 113 - protein, 452 aa, Artificial Sequence
  Synthetic Exemplary chimeric D2E7 antiTNF- alpha variable heavy chain and variable canine IgG-D Fc (F00; Protein A+; C1q+; CD16+) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPAPEMLGGPSVFIFPPKPKDTLFIARTPEITCVVVDLDPEDPEVQISWFVDGKEVHTAKTQPREEQFNSTYRVVSVLPIGHQDWLTGKEFKCKVNNKALPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 114 - protein, 471 aa, Artificial Sequence
  Synthetic Exemplary chimeric D2E7 antiTNF- alpha variable heavy chain and variable canine IgG-D Fc (F00, Protein A+; C1q; CD16) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDTLFIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 115 - protein, 452 aa, Artificial Sequence
  Synthetic Exemplary chimeric D2E7 antiTNF- alpha variable heavy chain and variable canine IgG-D Fc (F00, Protein A+; C1q; CD16) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDTLFIARTPEITCVVLDLGREDPEVQISWFVDGKEVHTAKTQPREQQFNSTYRVVSVLPIGHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALHNHYTDLSLSHSPGK

SEQ ID NO: 116 - protein, 237 aa, Artificial Sequence
  Synthetic Exemplary chimeric D2E7 antiTNF- alpha variable light chain and feline kappa constant light chain with leader sequence
METDTLLLWVLLLWVPGSTGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 117 - protein, 217 aa, Artificial Sequence
  Synthetic Exemplary chimeric D2E7 antiTNF- alpha variable light chain and feline kappa constant light chain without leader sequence
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 118 - protein, 475 aa, Artificial Sequence
  Synthetic Exemplary chimeric D2E7 antiTNF- alpha variable heavy chain and wild-type feline IgG1b Fc with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 119 - protein, 456 aa, Artificial Sequence
  Synthetic Exemplary chimeric D2E7 antiTNF- alpha variable heavy chain and wild-type feline IgG1b Fc without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 120 - protein, 475 aa, Artificial Sequence
  Synthetic Exemplary chimeric D2E7 antiTNF- alpha variable heavy chain and variant feline IgG1b Fc (YTE) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLYITREPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 121 - protein, 456 aa, Artificial Sequence
  Synthetic Exemplary chimeric D2E7 antiTNF- alpha variable heavy chain and variant feline IgG1 Fc (YTE) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLYITREPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 122 - protein, 234 aa, Artificial Sequence
  Synthetic Exemplary chimeric D2E7 antiTNF- alpha variable light chain and equine kappa constant light chain with leader sequence
METDTLLLWVLLLWVPGSTGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRDDAKPSAFIFPPSSEELSSGSASVVCLVYGFYPSGATINWKVDGLAKTSSFHSSLTEQDSKDNTYSLSSTLTLPKADYEAHNVYACEVSHKTLSSPLVKSFNREDC

SEQ ID NO: 123 - protein, 214 aa, Artificial Sequence
  Synthetic Exemplary chimeric D2E7 antiTNF- alpha variable light chain and equine kappa constant light chain without leader sequence
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRDDAKPSAFIFPPSSEELSSGSASVVCLVYGFYPSGATINWKVDGLAKTSSFHSSLTEQDSKDNTYSLSSTLTLPKADYEAHNVYACEVSHKTLSSPLVKSFNREDC

SEQ ID NO: 124 - protein, 477 aa, Artificial Sequence
  Synthetic Exemplary chimeric D2E7 antiTNF- alpha variable heavy chain and wild-type equine IgG1 Fc with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 125 - protein, 458 aa, Artificial Sequence
  Synthetic Exemplary chimeric D2E7 antiTNF- alpha variable heavy chain and wild-type equine IgG1 Fc without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLMITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 126 - protein, 477 aa, Artificial Sequence
  Synthetic Exemplary chimeric D2E7 antiTNF- alpha variable heavy chain and variant equine IgG1 Fc (F00) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLFITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 127 - protein, 458 aa, Artificial Sequence
  Synthetic Exemplary chimeric D2E7 antiTNF- alpha variable heavy chain and variant equine IgG1 Fc (F00) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLFITRTPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 128 - protein, 477 aa, Artificial Sequence
  Synthetic Exemplary chimeric D2E7 antiTNF- alpha variable heavy chain and variant equine IgG1 Fc (Y0E) with leader sequence
MAVLGLLLCLVTFPSCVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLYITREPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 129 - protein, 458 aa, Artificial Sequence
  Synthetic Exemplary chimeric D2E7 antiTNF- alpha variable heavy chain and variant equine IgG1 Fc (Y0E) without leader sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPKVFALAPGCGTTSDSTVALGCLVSGYFPEPVKVSWNSGSLTSGVHTFPSVLQSSGFYSLSSMVTVPASSWTSETYICNVVHAASNFKVDKRIEPIPDNHQKVCDMSKCPKCPAPELLGGPSVFIFPPNPKDTLYITREPEVTCVVVDVSQENPDVKFNWYMDGVEVRTATTRPKEEQFNSTYRVVSVLRIQHQDWLSGKEFKCKVNNQALPQPIERTITKTKGRSQEPQVYVLAPHPDELSKSKVSVTCLVKDFYPPEINIEWQSNGQPELETKYSTTQAQQDSDGSYFLYSKLSVDRNRWQQGTTFTCGVMHEALHNHYTQKNVSKNPGK

SEQ ID NO: 130 - protein, 124 aa, Canis lupus familiaris
  misc_feature (1)..(124): FIG. 1A - partial sequence of canine FcRn
KDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKD

SEQ ID NO: 131 - protein, 111 aa, Canis lupus familiaris
  misc_feature (1)..(111): FIG. 1A - partial sequence of canine FcRn
PKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSR

SEQ ID NO: 132 - protein, 122 aa, Canis lupus familiaris
  misc_feature (1)..(122): FIG. 1A - partial sequence of canine FcRn
PSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELS

SEQ ID NO: 133 - protein, 71 aa, Canis lupus familiaris
  misc_feature (1)..(71): FIG. 8 - partial sequence of canine FcRn
PPKPKDTLLIARTPEVTCVVVDLDPEDPEVQISWFVDGKQMQTAKTQPREEQFNGTYRVVSVLPIGHQDWL

SEQ ID NO: 134 - protein, 71 aa, Canis lupus familiaris
  misc_feature (1)..(71): FIG. 8 - partial sequence of canine FcRn
IDVEWQSNGQQEPESKYRTTPPQLDEDGSYFLYSKLSVDKSRWQRGDTFICAVMHEALHNHYTQESLSHSP